(19) United States
(12) Patent Application Publication
(10) Pub. No.: US 2025/0074895 A1
(43) Pub. Date: Mar. 6, 2025

(54) CDK2 INHIBITORS AND METHODS OF MAKING AND USING SAME

(71) Applicant: BLUEPRINT MEDICINES CORPORATION, Cambridge, MA (US)

(72) Inventors: Richard Vargas, Cambridge, MA (US); Emanuele Perola, Cambridge, MA (US); Philip D. Ramsden, Cambridge, MA (US); Steven Mark Wenglowsky, Cambridge, MA (US); Douglas Wilson, Ayer, MA (US)

(21) Appl. No.: 18/710,715

(22) PCT Filed: Nov. 19, 2022
(86) PCT No.: PCT/US2022/080199

§ 371 Date: May 16, 2024

Related U.S. Application Data
(60) Provisional application No. 63/281,508, filed on Nov. 19, 2021.

(30) Foreign Application Priority Data
Nov. 18, 2022 (WO) PCT/US2022/080165

(51) Int. Cl. C07D 403/12; A61K 31/4439; A61K 31/497; A61K 31/501; A61K 31/506; A61K 31/53; A61K 31/5377; C07D 401/12; C07D 405/14; C07D 413/14

(52) U.S. Cl. CPC C07D 403/12; A61K 31/4439; A61K 31/497; A61K 31/501; A61K 31/506; A61K 31/53; A61K 31/5377; C07D 401/12; C07D 405/14; C07D 413/14

(57) ABSTRACT

The present disclosure provides a compound represented by structural formula (I): or a pharmaceutically acceptable salt thereof useful for treating a cancer.

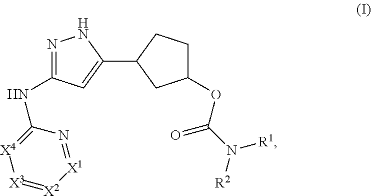

### CROSS REFERENCE TO RELATED APPLICATIONS

This application claims the benefit of and priority to U.S. Provisional Patent Application No. 63/281,508, filed on Nov. 19, 2021, the disclosure of which is hereby incorporated by reference in its entirety for all purposes.

### BACKGROUND

Cyclin-Dependent Kinase (CDK) are serine/threonine protein kinases that have a central role in cell cycle progression. CDK levels remain relatively constant throughout the cell cycle, and it is the selective activation of specific CDKs allows for the proper ordering of the steps in cell cycle progression. Activation of CDKs requires heterodimerization with regulatory subunits known as cyclins. Cell cycle deregulation is a common feature of human cancer.

Cyclin-dependent kinase 2 (Cdk2) participates in a range of biological activities. CDK2 is a key cell cycle regulator, active from the late G₁-phase and throughout the S-phase. CDK2 is involved in DNA damage response (DDR) through the homologous recombination (HR) pathway. CDK2 also regulates aspects of apoptotic pathways. Cyclin E1 (CCNE1), cyclin E2 (CCNE2), cyclin A1 (CCNA1), and cyclin A2 (CCNA2), along with p21Cip1/Waf1, p27Kip1 and p57Kip2 (the cyclin dependent kinase inhibitors of the cyclin-CDK2 complex) are the main regulators of CDK2 activity. In cancer, dysregulation of the binding of CDK2 by cyclin E1, E2, A1, or A2 or the activity of the cyclin-dependent kinase inhibitor proteins may occur. (See S. Tadesse et al., Drug Discovery Today, Volume 25, Number 2 Feb. 2020)

The dysregulation of CDK2 can occur through several mechanisms. Amplification or overexpression of CCNE1 has been identified occurring in ovarian and breast cancer (See Scaltriti, M. et al., Proc. Natl Acad. Sci. USA 108, 3761-3766 (2011) and Etemadmoghadam, D. et al. Proc. Natl Acad. Sci. USA 110, 19489-19494 (2013). Poor outcomes in gastric, endometrial, and other cancers have been associated with overexpression or amplification of CCNE1 (See Ooi et al. Hum Pathol. (2017) 61:58-67, and Noske et al., Oncotarget (2017) 8: 14794-14805).

While these findings indicate CDK2 is a potential target for cancers with deregulated CDK2 activity, no agents targeting CDK2 have been approved to date. Therefore, there is a need to develop new CDK2 inhibitors.

### SUMMARY

Disclosed herein are compounds which are effective inhibitors of CDK2 (see Synthetic Examples 1-108). In particular, it has been demonstrated that compounds of the present disclosure effectively inhibit CDK2 and can be used treat various cancers. For example, disclosed compounds are selective CDK2 inhibitors. i.e., disclosed compounds have no or low activity against CDK1. Advantages associated with such selectivity may include facilitating efficacious dosing and reducing CDK1-mediated on-target toxicities. Certain disclosed compounds may have the advantage of having high, significant microsomal stability and/or favorable toxicity profiles as compared to other non-kinase targets.

In one aspect, the present disclosure provides a compound represented by the following structural Formula (I):

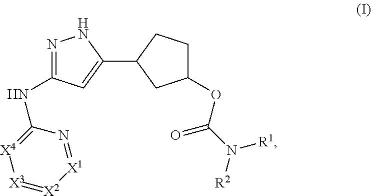

or a pharmaceutically acceptable salt thereof, the definition of each variable is provided below.

In another aspect, the present disclosure provides a compound represented by the following structural Formula (I-1):

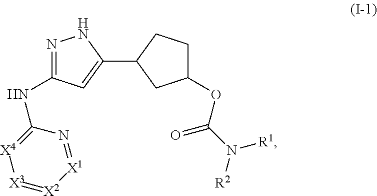

or a pharmaceutically acceptable salt thereof the definition of each variable is provided below.

In another aspect, the present disclosure provides a pharmaceutical composition comprising a pharmaceutically acceptable carrier or diluent and one or more of the compounds disclosed herein, or a pharmaceutically acceptable salt thereof (a “pharmaceutical composition of the disclosure”).

The present disclosure provides a method of treating a subject with cancer, comprising administering to the subject an effective amount of a compound of the disclosure (e.g., a compound of Formula (I)) or a pharmaceutically acceptable salt thereof or a pharmaceutical composition of the disclosure. In one embodiment, the cancer is uterine cancer (including uterine carcinosarcoma (UCS), uterine corpus endometrial carcinoma (UCEC)), endometrial cancer, breast cancer (including breast invasive carcinoma (BRCA), TNBC (triple negative breast cancer), HR+ breast cancer (hormone receptor positive breast cancer), ER+ breast cancer (estrogen receptor positive breast cancer), HR+HER2− breast cancer (hormone receptor positive, human epidermal growth factor 2 negative breast cancer), ER+HER2− breast cancer (estrogen receptor positive, human epidermal growth factor 2 negative breast cancer), HER2− breast cancer (human epidermal growth factor 2 negative breast cancer), HER2−low breast cancer (human epidermal growth factor 2 low breast cancer), and HER2+ breast cancer (human epidermal growth factor 2 positive breast cancer), ovarian cancer (e.g., ovarian serous cystadenocarcinoma (OV)), stomach cancer (including stomach adenocarcinoma (STAD)), gastric cancer (including gastrointestinal stromal tumor), colorectal cancer, pancreatic cancer (including pancreatic adenocarcinoma (PAAD), kidney cancer, head and neck cancer, liver cancer, prostate cancer, skin cancer, leukemia (including AML (acute myeloid leukemia)), lymphoma (including B-cell lymnphoma), mvelodysplastic syndromes (MDS), myeloproliferative neoplasms (MPN), sarcoma (SARC), esophageal cancer (including esophageal carcinoma (ESCA)), bladder cancer (including bladder urothelial carcinoma), lung cancer (including lung squamous carcinoma and non-small cell lung cancer, e.g., EGFRm (epidermal growth factor receptor mutant)+ non-small cell lung cancer), cholangiocarcinoma, adrenocortical carcinoma (ACC), or mesothelioma. In some embodiments, the cancer is breast cancer. In one embodiment, the subject has CCNE1 amplified advanced/relapsed tumors. In one embodiment, the subject has CCNE1 amplified platinum-resistant or platinum-refectory ovarian cancer. In one embodiment, the subject has endometrial cancer (with prior platinum therapy, e.g., wherein the patient has been previously treated with a platinum therapy) that has progressed following 2 or more lines of therapies (including the platinum therapy). In one embodiment, the subject has CCNE1 amplified endometrial cancer that has failed 2 or more lines of therapies (which may include a prior platinum therapy). In one embodiment, the subject has gastric cancer (with prior platinum therapy e.g., wherein the patient has been previously treated with a platinum therapy) that has progressed following 2 or more lines of therapies (including the platinum therapy). In one embodiment, the subject has ER+ HER− breast cancer that has progressed despite treatment with one or more CDK4/6 inhibitors.

In one embodiment, the cancer as described herein to be treated (e.g., the cancer as described in paragraphs [0010], 100201, [0077]-[0086], [0088], and [0090]-[0105], e.g. breast cancer) has CCNE1 amplification and/or overexpression.

In one embodiment, the cancer as described herein to be treated (e.g., the cancer as described in paragraphs [0010], [0020], [0077]-[0086], [0088], and [0090]-[0105], e.g. breast cancer) does not have a CCNE1 amplification and/or overexpression.

The treatment method disclosed herein further comprises administering to the subject an effective amount of palbociclib (e.g., Ibrance®), ribociclib, abemaciclib, tamoxifen, letrozole, olaparib (e.g., Lynparza®), niraparib, carboplatin, cisplatin, paclitaxel, gemcitabine, megestrol acetate, medroxyprogesterone acetate, capecitabine (e.g., Xeloda®), regorafenib (e.g., Stivarga®), afatinib (e.g., Gilotrif®), osimertinib (e.g., Tagrisso®), gefitinib (e.g., Iressa®)), erlotinib (e.g., Tarceva®), ramucirumab (e.g., Cyramza®), an EGFR inhibitor, pralsetinib, ABT-263 (navitoclax), MK-1775 (adavosertib), BAY-1895344, berzosertib, ceralasertib, SRA-737, LY2603618 (rabusertib), or trastuzumab (e.g., Herceptin®), or combinations thereof. The EGFR inhibitor may be selected from afatinib, osinertinib, lapatinib, erlotinib, dacomitinib, poziotinib, neratinib, gefitinib JBJ-04-125-02, alflutinib (AST 2818), aumolertinib (formerly aimonertinib) (HS10296), BBT-176, BI-4020, BPI-361175, BPI-D0316, CH7233163, gilitertinib, icotinib, JND-3229, lazertinib, nazartinib (EGF 816), avitinib, PCC-0208027, rezivertinib (BPI-7711), TQB3804, zorifertinib (AZ-3759), or DZD9008; an EGFR antibody such as cetuximab, panitumumab, necitumumab, HLX07, JMT101; or a bispecific EGFR and MET antibody (e.g., amivantamab ((JNJ-61186372, JNJ-372)).

The present disclosure also provides a method of inhibiting CDK2 in a subject in need thereof, comprising administering to the subject an effective amount of a compound of the disclosure (e.g., a compound of Formula (I) or Formula (I-1)) or a pharmaceutically acceptable salt thereof or a pharmaceutical composition of the disclosure.

The present disclosure also provides the use of an effective amount of a compound of the disclosure (e.g., a compound of Formula (I) or Formula (I-1)), or a pharmaceutically acceptable salt thereof, or a pharmaceutical composition of the disclosure, for the preparation of a medicament for the treatment of cancers.

In another aspect, provided herein is a compound of Formula (I) or Formula (I-1), or a pharmaceutically acceptable salt thereof, or a pharmaceutical composition of the disclosure for use in treating cancers.

In one aspect, the present disclosure provides a method of treating a subject having, or at risk of developing, a disease or disorder associated with CDK2 comprising administering to the subject a therapeutically effective amount of a compound disclosed herein, or a pharmaceutically acceptable salt thereof, or a pharmaceutical composition disclosed herein, wherein the subject has an amplification of the CCNE1 gene and/or have an expression level of CCNE1 higher than a control expression level of CCNE1 In some embodiments, the disease or disorder associated with CDK2 is cancer.

The present disclosure also provides a method of treating a subject having, or at risk of developing, a disease or disorder associated with CDK2, comprising administering to the subject a therapeutically effective amount of a compound disclosed herein, or a pharmaceutically acceptable salt thereof, or a pharmaceutical composition disclosed herein, wherein the subject has an amplification of the CCNE1 gene and/or have an expression level of CCNE1 similar to a control expression level of CCNE1. In some embodiments, the disease or disorder associated with CDK2 is cancer.

Also provided herein is a method of treating a patient having an amplified expression level of CCNE1 and suffering from, or at risk of developing, a solid tumor cancer, comprising administering to the patient a therapeutically effective amount of a compound disclosed herein, or a pharmaceutically acceptable salt thereof, or a pharmaceutical composition disclosed herein.

The contemplated solid tumor cancer may be at least one of: uterine cancer (including uterine carcinosarcoma, uterine corpus endometrial carcinoma (UCEC)), endometrial cancer, breast cancer (including breast invasive carcinoma, TNBC (triple negative breast cancer), ER (estrogen receptor)+HER2 (human epidermal growth factor 2)− breast cancer, HR (hormone receptor)+HER2 (human epidermal growth factor 2)− breast cancer, HER2− breast cancer and HER2+ breast cancer), ovarian cancer (e.g., ovarian serous cystadenocarcinoma), stomach cancer (including stomach adenocarcinomna), gastric cancer (including gastrointestinal stromal tumor), colorectal cancer, pancreatic cancer, kidney cancer, head and neck cancer, liver cancer, prostate cancer, skin cancer, lymphoma (including B-cell lymphoma), sarcoma, esophageal cancer (including esophageal carcinoma and esophageal adenocarcinoma), bladder cancer (including bladder urothelial carcinoma (BLCA)), lung cancer (including lung squamous carcinoma and non-small cell lung cancer, e.g., EGFRm (epidermal growth factor receptor mutant)+ non-small cell lung cancer), cholangiocarcinoma, adrenocortical carcinoma, or mesothelioma.

### DETAILED DESCRIPTION

### Definitions

The term “halo” as used herein means halogen and includes chloro, fluoro, bromo and iodo.

The term “alkyl” used alone or as part of a larger moiety, such as “alkoxy” or “haloalkyl” and the like, means saturated aliphatic straight-chain or branched monovalent hydrocarbon radical. Unless otherwise specified, an alkyl group typically has 1-4 carbon atoms, i.e. (C₁-C₄)alkyl. As used herein, a “(C₁-C₄)alkyl” group means a radical having from 1 to 4 carbon atoms in a linear or branched arrangement. Examples include methyl, ethyl, n-propyl, iso-propyl, and the like.

The term “alkoxy” means an alkyl radical attached through an oxygen linking atom, represented by —O-alkyl. For example, “(C₁-C₄)alkoxy” includes methoxy, ethoxy, propoxy, and butoxy.

The term “cycloalkyl” refers to a monocyclic saturated hydrocarbon ring system. Unless otherwise specified, cycloalkyl has from 3-6 carbon atoms. For example, a C₃-C₆ cycloalkyl includes cyclopropyl, cyclobutyl, cyclopentyl and cyclohexyl. Unless otherwise described, a “cycloalkyl” has from three to six carbon atoms.

The term “heterocyclyl” or “heterocyclic” refers to a radical of a 4- to 6-membered non-aromatic ring system having ring carbon atoms and 1 to 2 ring heteroatoms, wherein each heteroatom is independently selected from nitrogen, quaternary nitrogen, oxidized nitrogen (e.g., NO), oxygen, and sulfur, including sulfoxide and sulfone (“4-12 membered heterocyclyl. In heterocyclyl groups that contain one or more nitrogen atoms, the point of attachment can be a carbon or nitrogen atom, as valency permits. Exemplary heterocyclyl groups include azetidinyl, oxetanyl, thietanyl, tetrahydrofuranyl, pyrrolidinyl, piperidinyl, tetrahydropyranyl, piperazinyl, morpholinyl, azepanyl, oxepanyl, thiepanyl, tetrahydropyridinyl, and the like.

### Compounds of the Present Disclosure

Disclosed herein are embodiments of compounds having a general structure of Formula (I) or Formula (I-1). The present invention provides a compound of the present invention or a pharmaceutically acceptable salt thereof for use in the treatment of cancer. These compounds are selective inhibitors of CDK2.

In a first embodiment, the present disclosure provides a compound represented by the following structural Formula (I-1):

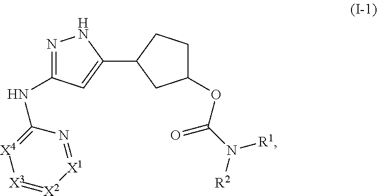

or a pharmaceutically acceptable salt thereof, wherein
R¹ is C₁-C₆alkyl, C₃-C₆cycloalkyl, or 4 to 6 membered heterocyclyl, wherein the C₁-C₆alkyl is optionally substituted with 1 to 4 groups independently selected from halo and C₃-C₆cycloalkyl, wherein the cycloalkyl or heterocyclyl is optionally substituted with 1 to 4 groups independently selected from halo and C₁-C₄alkyl optionally substituted with 1 to 4 halo, wherein the heterocyclyl includes 1 or 2 heteroatoms independently selected from O, N, or S;
R² is H or C₁-C₆ alkyl; or
R¹ and R², taken together with the nitrogen atom to which they are attached, form a 4 to 6 membered heterocyclyl, wherein the heterocyclyl optionally includes 1 or 2 heteroatoms independently selected from O, N, or S in addition to the nitrogen atom attached to R¹ and R², and is optionally substituted with 1 to 4 groups independently selected from the group consisting of halo, CO₂R^{d}, CN, C₁-C₄alkyl, and C₁-C₄alkoxy, wherein the C₁-C₄alkyl and C₁-C₄alkoxy are each optionally substituted with 1 to 4 halo;
X¹ is N or CR³;
X² is N or CR³;
X³ is N or CR³;
X⁴ is N or CR³; wherein at least 2 of X¹, X², X³, and X⁴ are CR³;
R³, independently for each occurrence, is selected from the group consisting of H, halo, CN, OH, NR^{a}R^{b}, SO_{w}R^{c}, C₁-C₄alkyl, C₁-C₄alkoxy, and C₃-C₆cycloalkyl, wherein the alkyl or alkoxy is optionally substituted with t to 4 groups independently selected from halo, OR^{3a}, and C₃-C₆cycloalkyl, and the C₃-C₆cycloalkyl is optionally substituted with 1 to 4 groups independently selected from halo and OH;
w is 0, 1, or 2;
R^{a} is independently for each occurrence H or C₁-C₄alkyl optionally substituted by one, two or three halos;
R^{b} is independently for each occurrence H or C₁-C₄alkyl optionally substituted by one, two or three halos;
R^{c} is independently for each occurrence C₁-C₄alkyl optionally substituted by one, two or three halos;
R^{d} is independently for each occurrence H or C₁-C₄alkyl optionally substituted by one, two or three halos; and
R^{3a} for each occurrence is H or C₁₋₄alkyl optionally substituted by one, two or three halos.

In another embodiment, the present disclosure provides a compound represented by the following structural Formula (I),

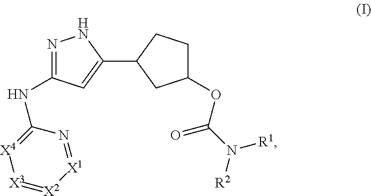

or a pharmaceutically acceptable salt thereof, wherein
R¹ is C₁-C₆alkyl, C₃-C₆cycloalkyl, or 4 to 6 membered heterocyclyl, wherein the cycloalkyl or heterocyclyl is optionally substituted with 1 to 4 groups selected from halo and C₁-C₄alkyl optionally substituted with 1 to 4 halo, wherein the heterocyclyl includes 1 or 2 heteroatoms selected from O, N, or S;
R² is H or C₁-C₅ alkyl, or
R¹ and R², taken together with the nitrogen atom to which they are attached, form a 4 to 6 membered heterocyclyl, optionally substituted with 1 to 4 groups selected from halo or C₁-C₄alkyl,
X¹ is N or CR³;
X² is N or CR³;
X³ is N or CR³;
X⁴ is N or CR³; wherein at least 2 of X¹, X², X³, and X⁴ are CR³;
R³, independently for each occurrence is selected from the group consisting of H, halo, CN, OH, NR^{a}R^{b}, SO_{w}R^{c}, C₁-C₄alkyl, C₁-C₄alkoxy, and C₃-C₆cycloalkyl, wherein the alkyl or alkoxy is optionally substituted with 1 to 4 groups independently selected from halo, OR^{3a}, and C₃-C₆cycloalkyl, and the C₃-C₆cycloalkyl is optionally substituted with 1 to 4 groups independently selected from halo and OH;
w is 0, 1, or 2;
R^{a} is independently for each occurrence H or C₁-C₄alkyl optionally substituted by one, two or three halos;
R^{b} is independently for each occurrence H or C₁-C₄alkyl optionally substituted by one, two or three halos;
R^{c} is independently for each occurrence C₁-C₄alkyl optionally substituted by one, two or three halos; and
R^{3a} for each occurrence is H or C₁₋₄alkyl optionally substituted by one, two or three halos.

In some embodiments of Formula (I) and Formula (I-1), R¹ is C₁-C₄ Cycloalkyl, wherein the C₃-C₄ cycloalkyl is optionally substituted with C₁-C₂ alkyl. In some embodiments of Formula (I) and Formula (I-1). R¹ is C₁-C₂ alkyl. In some embodiments of Formula (I) and Formula (I-1), R¹ is a 4 to 6 membered heterocyclyl containing 1 oxygen or 1 nitrogen, optionally substituted with halo or C₁-C₄alkyl optionally substituted with 1 to 4 halo.

In some embodiments of Formula (I) and Formula (I-1), R¹ is cyclopropyl. In some embodiments of Formula (I) and Formula (I-1), R¹ is cyclopropyl substituted with a methyl.

In some embodiments of Formula (I) and Formula (I-1), R¹ and R², taken together with the nitrogen atom to which they are attached, combine to form an azetidine. In certain embodiments of Formula (I) and Formula (I-1), the azetidine is substituted with 1 or 2 methyl.

In certain embodiments of Formula (I) and Formula (I-1), X¹ is N. In certain embodiments of Formula (I) and Formula (I-1), X¹ is CR³.

In certain embodiments of Formula (I) and Formula (I-1), X² is N. In certain embodiments of Formula (I) and Formula (I-1), X² is CR³.

In certain embodiments of Formula (I) and Formula (I-1), X³ is N. In certain embodiments of Formula (I) and Formula (I-1), X³ is CR³

In certain embodiments of Formula (I) and Formula (I-1) V is N. In certain embodiments of Formula (I) and Formula (I-1), X⁴ is CR³.

In certain embodiments of Formula (I) and Formula (I-1), R is selected from the group consisting of H, methyl, chloro, fluoro, —CN, —CF₃, —CHF₂,

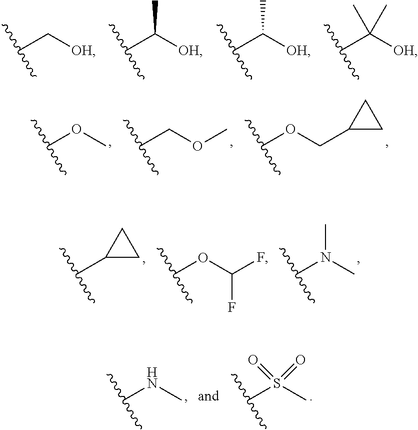

In certain embodiments of Formula (I) and Formula (I) R^{a} is H. In certain embodiments, R^{a} is methyl.

In certain embodiments of Formula (I) and Formula (I-1), R^{b} is H. In certain embodiments, R^{b} is methyl.

In certain embodiments of Formula (I) and Formula (I-1), R^{b} is methyl and R^{b} is methyl.

In some embodiments of Formula (I) and Formula (I-1), R² is H. In some embodiments of Formula (I) and Formula (I-1), R is C₁-C₆ alkyl.

In some embodiments of Formula (I) and Formula (I-1), R³ is methyl.

In some embodiments of Formula (I) and Formula (I-1), the compound of Formula (I) or Formula (I-1) is selected from the group consisting of: Formula IIA, IIB, IIC, IID, IIE, and IIF:

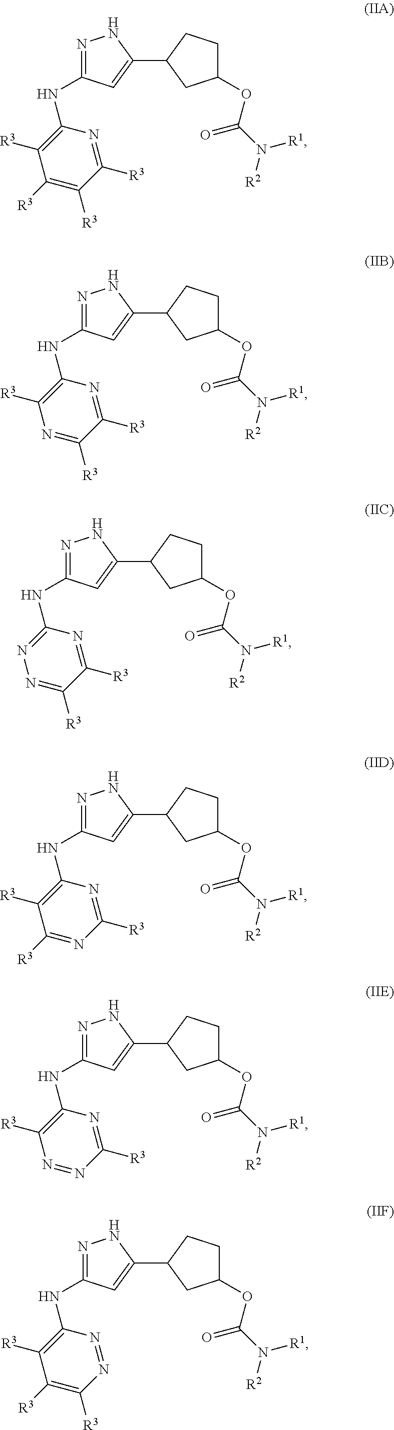

or a pharmaceutically acceptable salt thereof.

In some embodiments of Formula (I) and Formula (I-1), R is C₁-C₆alkyl and R² is H. In some embodiments of Formula (I) and Formula (I-1), R₁ is C₁-C₆alkyl optionally substituted with 1 to 4 groups selected from halo and C₃-C₆cycloalkyl and R₂ is H. In some embodiments of Formula (I) and Formula (I-1), R₁ is C₁-C₆alkyl optionally substituted with 1 to 4 groups selected from halo. In some embodiments of Formula (I) and Formula (I-1). R₁ is C₁-C₆alkyl optionally substituted with C₃-C₆cycloalkyl. In some embodiments of Formula (I) and Formula (I-1), R¹ is C₃₋₄cycloalkyl, optionally substituted by methyl. In some embodiments of Formula (I) and Formula (I-1). R¹ is C₃₋₄cycloalkyl, optionally substituted by methyl or ethyl, wherein the methyl or ethyl is optionally substituted with 1 to 4 halo. In some embodiments of Formula (I) and Formula (I-1), R¹ is oxetanyl, tetrahydrofuranyl, tetrahydropyranyl, azetidinyl, pyrrolidinyl, or piperidinyl, optionally substituted by methyl or CF₃.

In some embodiments of Formula (I) and Formula (I-1), the compound of Formula (I) or Formula (I-1) is selected from the group consisting of: Formula IIIA, IIIB, IIIC, IIID, IIIE, and IIIF:

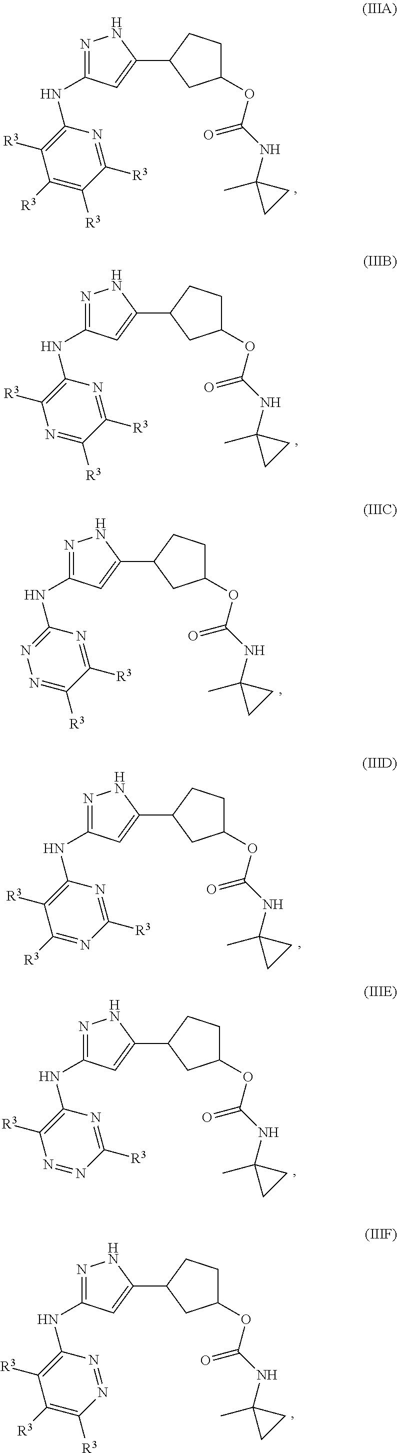

or a pharmaceutically acceptable salt thereof.

In some embodiments of Formula (I) and Formula (I-1), R¹ and R² together with the nitrogen to which they are attached, form a 4-6 membered heterocyclyl, optionally substituted on a free carbon by one or two methyl groups. In some embodiments of Formula (I) and Formula (I-1), R¹ and R², together with the nitrogen to which they are attached, form a 4-5 membered heterocyclyl, optionally substituted on a free carbon by one or two methyl groups. In some embodiments of Formula (I) and Formula (I-1), R¹ and R², together with the nitrogen to which they are attached, form a 4-6 membered heterocyclyl, wherein the heterocyclyl optionally includes 1 or 2 heteroatoms independently selected from O, N, or S in addition to the nitrogen atom attached to R¹ and R² and is optionally substituted on a free carbon or nitrogen by CN, CO₂C(CH₃)₃, OCH₃, or one or two methyl groups optionally substituted with 1 to 4 halo.

In some embodiments of Formula (J) and Formula (I-1), the compound of Formula (I) or Formula (I-1) is selected from the group consisting of Formula IVA, IVB, IVC, IVD, IVE, and IVF:

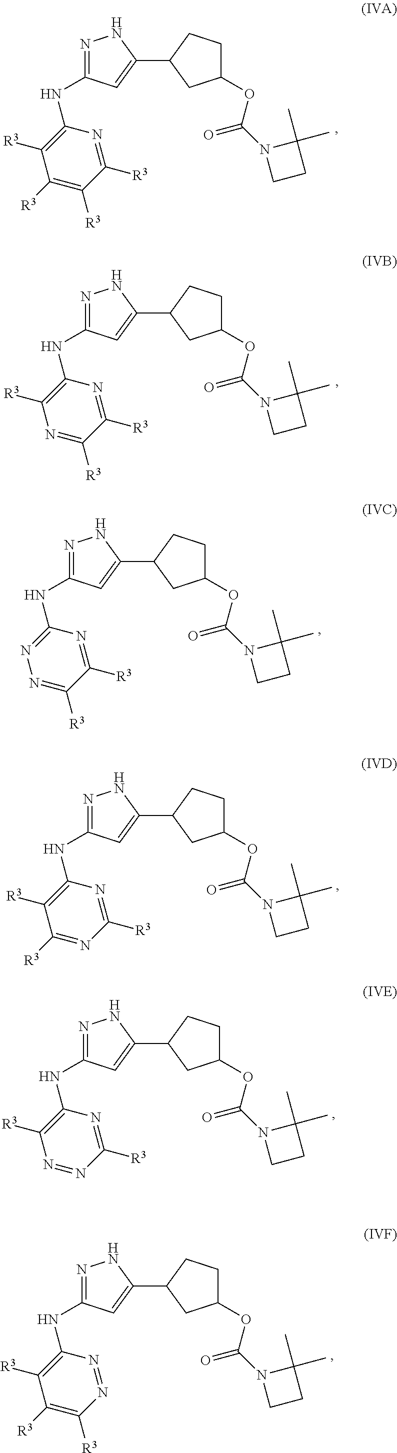

or a pharmaceutically acceptable salt thereof.

In some embodiments of Formula (I) and Formula (I-1), each R³ is independently selected from the group consisting of H, —OH, halo, CN, NR^{a}R^{b}, SO₂R^{c}, C₁-C₄alkyl, and C₁-C₄alkoxyl, wherein the alkyl and alkoxy are each optionally substituted with 1 to 4 groups independently selected from halo, OR^{3a}, or cyclopropyl;
each R^{a} is independently selected for each occurrence from the group consisting of H methyl, or ethyl;
each R^{b} is independently selected for each occurrence from methyl and ethyl;
R^{3a} is independently selected for each occurrence from methyl or ethyl; and
R^{c} is selected for each occurrence from methyl or ethyl.

In one embodiment of Formula (I-1) or Formula (I), the compound is a compound or a pharmaceutically acceptable salt thereof selected from the following table:

| Compound | |
|---|---|
| number | Structure |
| 1 | 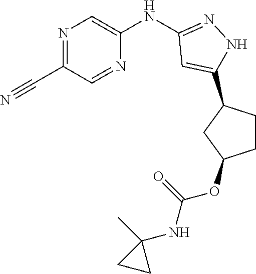 [SMILES: CC1(NC(=O)O[C@@H]2CC[C@H](c3cc(Nc4cnc(C#N)cn4)n[nH]3)C2)CC1] |
| 2 | 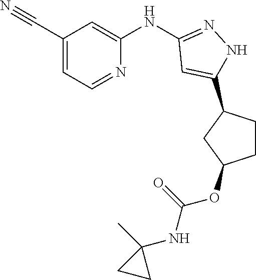 [SMILES: CC1(NC(=O)O[C@@H]2CC[C@H](c3cc(Nc4cc(C#N)ccn4)n[nH]3)C2)CC1] |
| 3 | 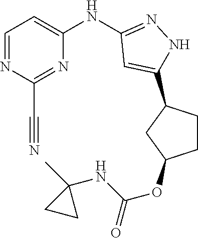 [SMILES: CC1(NC(=O)O[C@@H]2CC[C@H](c3cc(Nc4ccnc(C#N)n4)n[nH]3)C2)CC1] |
| 4 | 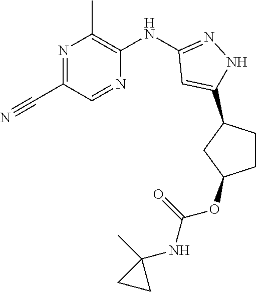 [SMILES: Cc1nc(C#N)cnc1Nc1cc([C@H]2CC[C@@H](OC(=O)NC3(C)CC3)C2)[nH]n1] |
| 5 | 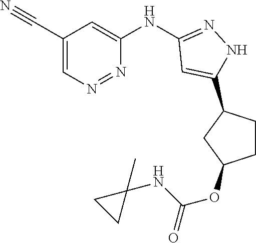 [SMILES: CC1(NC(=O)O[C@@H]2CC[C@H](c3cc(Nc4cc(C#N)cnn4)n[nH]3)C2)CC1] |
| 6 | 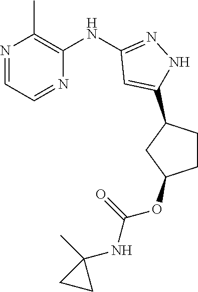 [SMILES: Cc1nccnc1Nc1cc([C@H]2CC[C@@H](OC(=O)NC3(C)CC3)C2)[nH]n1] |
| 7 | 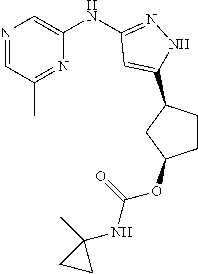 [SMILES: Cc1cncc(Nc2cc([C@H]3CC[C@@H](OC(=O)NC4(C)CC4)C3)[nH]n2)n1] |
| 8 | 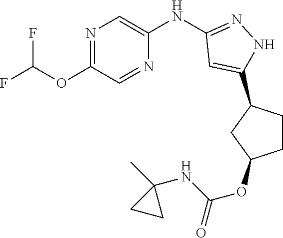 [SMILES: CC1(NC(=O)O[C@@H]2CC[C@H](c3cc(Nc4cnc(OC(F)F)cn4)n[nH]3)C2)CC1] |
| 9 | 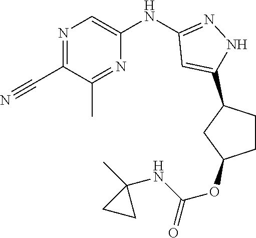 [SMILES: Cc1nc(Nc2cc([C@H]3CC[C@@H](OC(=O)NC4(C)CC4)C3)[nH]n2)cnc1C#N] |
| 10 | 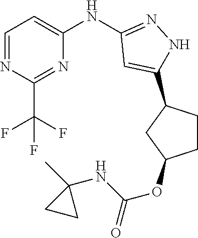 [SMILES: CC1(NC(=O)O[C@@H]2CC[C@H](c3cc(Nc4ccnc(C(F)(F)F)n4)n[nH]3)C2)CC1] |
| 11 | 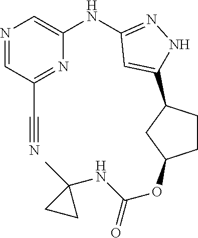 [SMILES: CC1(NC(=O)O[C@@H]2CC[C@H](c3cc(Nc4cncc(C#N)n4)n[nH]3)C2)CC1] |
| 12 | 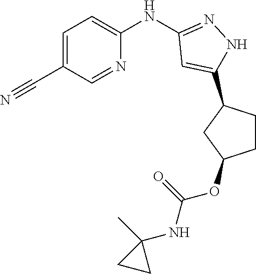 [SMILES: CC1(NC(=O)O[C@@H]2CC[C@H](c3cc(Nc4ccc(C#N)cn4)n[nH]3)C2)CC1] |
| 13 | 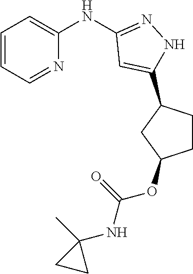 [SMILES: CC1(NC(=O)O[C@@H]2CC[C@H](c3cc(Nc4ccccn4)n[nH]3)C2)CC1] |
| 14 | 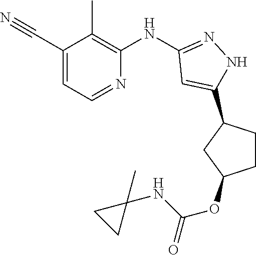 [SMILES: Cc1c(C#N)ccnc1Nc1cc([C@H]2CC[C@@H](OC(=O)NC3(C)CC3)C2)[nH]n1] |
| 15 | 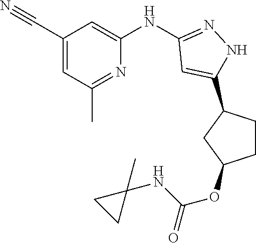 [SMILES: Cc1cc(C#N)cc(Nc2cc([C@H]3CC[C@@H](OC(=O)NC4(C)CC4)C3)[nH]n2)n1] |
| 16 | 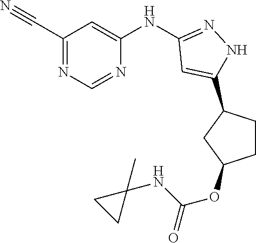 [SMILES: CC1(NC(=O)O[C@@H]2CC[C@H](c3cc(Nc4cc(C#N)ncn4)n[nH]3)C2)CC1] |
| 17 | 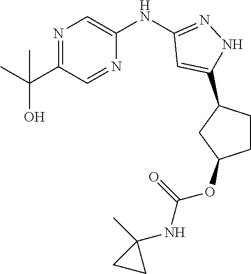 [SMILES: CC1(NC(=O)O[C@@H]2CC[C@H](c3cc(Nc4cnc(C(C)(C)O)cn4)n[nH]3)C2)CC1] |
| 18 | 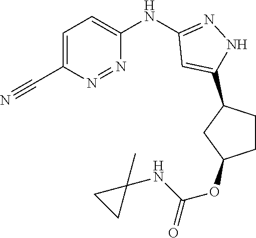 [SMILES: CC1(NC(=O)O[C@@H]2CC[C@H](c3cc(Nc4ccc(C#N)nn4)n[nH]3)C2)CC1] |
| 19 | 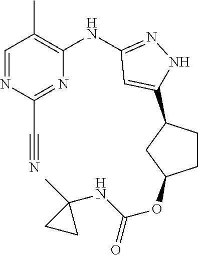 [SMILES: Cc1cnc(C#N)nc1Nc1cc([C@H]2CC[C@@H](OC(=O)NC3(C)CC3)C2)[nH]n1] |
| 20 | 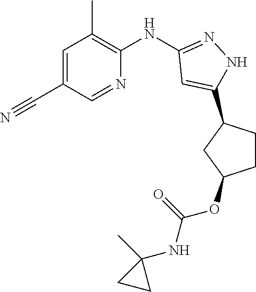 [SMILES: Cc1cc(C#N)cnc1Nc1cc([C@H]2CC[C@@H](OC(=O)NC3(C)CC3)C2)[nH]n1] |
| 21 | 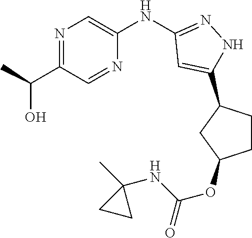 [SMILES: C[C@H](O)c1cnc(Nc2cc([C@H]3CC[C@@H](OC(=O)NC4(C)CC4)C3)[nH]n2)cn1] |
| | OR |
| | 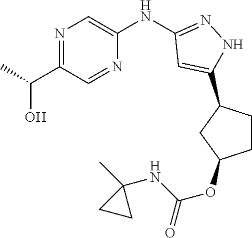 [SMILES: C[C@@H](O)c1cnc(Nc2cc([C@H]3CC[C@@H](OC(=O)NC4(C)CC4)C3)[nH]n2)cn1] |
| 22 | 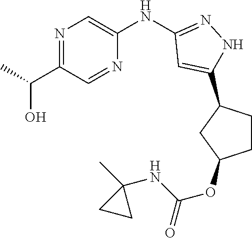 [SMILES: C[C@@H](O)c1cnc(Nc2cc([C@H]3CC[C@@H](OC(=O)NC4(C)CC4)C3)[nH]n2)cn1] |
| | OR |
| | 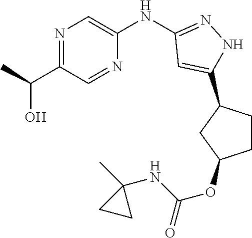 [SMILES: C[C@H](O)c1cnc(Nc2cc([C@H]3CC[C@@H](OC(=O)NC4(C)CC4)C3)[nH]n2)cn1] |
| 23 | 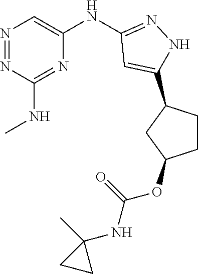 [SMILES: CNc1nncc(Nc2cc([C@H]3CC[C@@H](OC(=O)NC4(C)CC4)C3)[nH]n2)n1] |
| 24 | 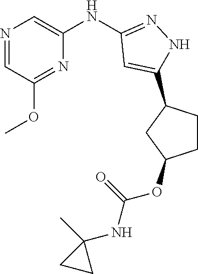 [SMILES: COc1cncc(Nc2cc([C@H]3CC[C@@H](OC(=O)NC4(C)CC4)C3)[nH]n2)n1] |
| 25 | 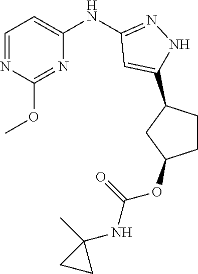 [SMILES: COc1nccc(Nc2cc([C@H]3CC[C@@H](OC(=O)NC4(C)CC4)C3)[nH]n2)n1] |
| 26 | 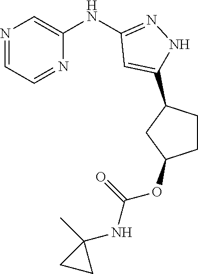 [SMILES: CC1(NC(=O)O[C@@H]2CC[C@H](c3cc(Nc4cnccn4)n[nH]3)C2)CC1] |
| 27 | 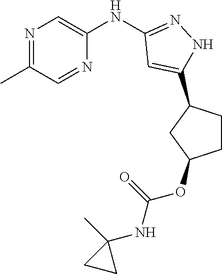 [SMILES: Cc1cnc(Nc2cc([C@H]3CC[C@@H](OC(=O)NC4(C)CC4)C3)[nH]n2)cn1] |
| 28 | 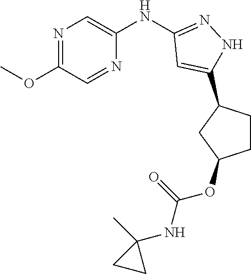 [SMILES: COc1cnc(Nc2cc([C@H]3CC[C@@H](OC(=O)NC4(C)CC4)C3)[nH]n2)cn1] |
| 29 | 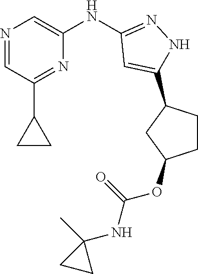 [SMILES: CC1(NC(=O)O[C@@H]2CC[C@H](c3cc(Nc4cncc(C5CC5)n4)n[nH]3)C2)CC1] |
| 30 | 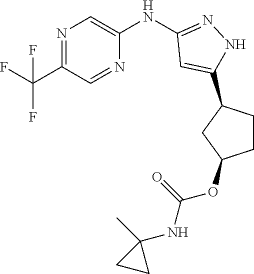 [SMILES: CC1(NC(=O)O[C@@H]2CC[C@H](c3cc(Nc4cnc(C(F)(F)F)cn4)n[nH]3)C2)CC1] |
| 31 | 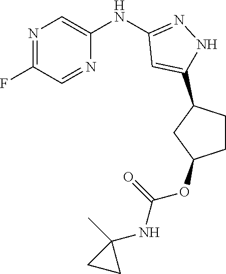 [SMILES: CC1(NC(=O)O[C@@H]2CC[C@H](c3cc(Nc4cnc(F)cn4)n[nH]3)C2)CC1] |
| 32 | 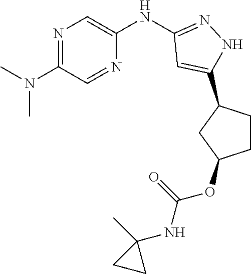 [SMILES: CN(C)c1cnc(Nc2cc([C@H]3CC[C@@H](OC(=O)NC4(C)CC4)C3)[nH]n2)cn1] |
| 33 | 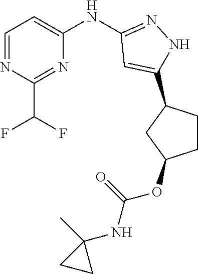 [SMILES: CC1(NC(=O)O[C@@H]2CC[C@H](c3cc(Nc4ccnc(C(F)F)n4)n[nH]3)C2)CC1] |
| 34 | 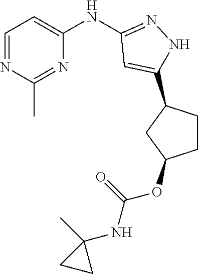 [SMILES: Cc1nccc(Nc2cc([C@H]3CC[C@@H](OC(=O)NC4(C)CC4)C3)[nH]n2)n1] |
| 35 | 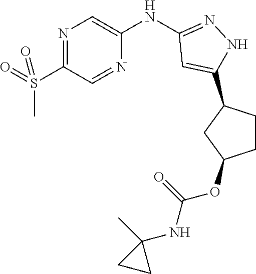 [SMILES: CC1(NC(=O)O[C@@H]2CC[C@H](c3cc(Nc4cnc(S(C)(=O)=O)cn4)n[nH]3)C2)CC1] |
| 36 | 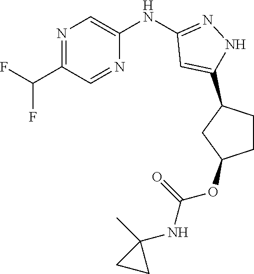 [SMILES: CC1(NC(=O)O[C@@H]2CC[C@H](c3cc(Nc4cnc(C(F)F)cn4)n[nH]3)C2)CC1] |
| 37 | 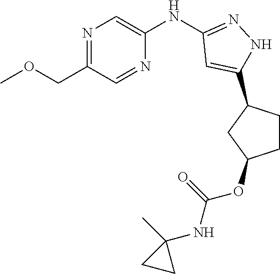 [SMILES: COCc1cnc(Nc2cc([C@H]3CC[C@@H](OC(=O)NC4(C)CC4)C3)[nH]n2)cn1] |
| 38 | 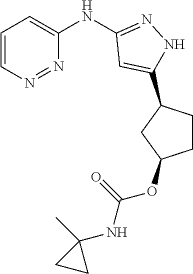 [SMILES: CC1(NC(=O)O[C@@H]2CC[C@H](c3cc(Nc4cccnn4)n[nH]3)C2)CC1] |
| 39 | 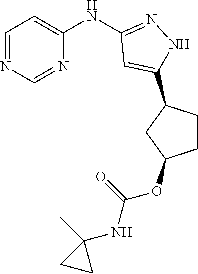 [SMILES: CC1(NC(=O)O[C@@H]2CC[C@H](c3cc(Nc4ccncn4)n[nH]3)C2)CC1] |
| 40 | 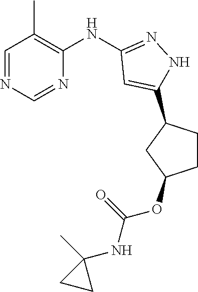 [SMILES: Cc1cncnc1Nc1cc([C@H]2CC[C@@H](OC(=O)NC3(C)CC3)C2)[nH]n1] |
| 41 | 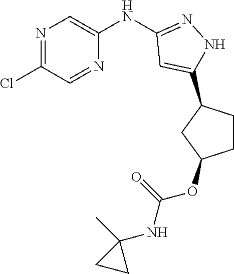 [SMILES: CC1(NC(=O)O[C@@H]2CC[C@H](c3cc(Nc4cnc(Cl)cn4)n[nH]3)C2)CC1] |
| 42 | 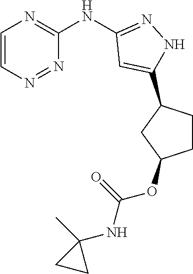 [SMILES: CC1(NC(=O)O[C@@H]2CC[C@H](c3cc(Nc4nccnn4)n[nH]3)C2)CC1] |
| 43 | 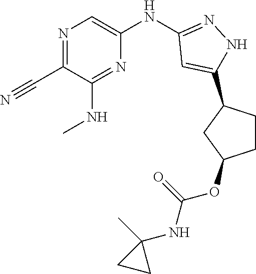 [SMILES: CNc1nc(Nc2cc([C@H]3CC[C@@H](OC(=O)NC4(C)CC4)C3)[nH]n2)cnc1C#N] |
| 44 | 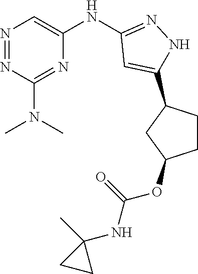 [SMILES: CN(C)c1nncc(Nc2cc([C@H]3CC[C@@H](OC(=O)NC4(C)CC4)C3)[nH]n2)n1] |
| 45 | 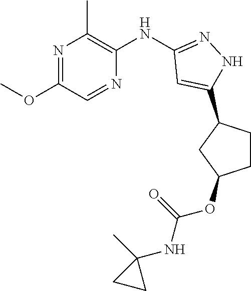 [SMILES: COc1cnc(Nc2cc([C@H]3CC[C@@H](OC(=O)NC4(C)CC4)C3)[nH]n2)c(C)n1] |
| 46 | 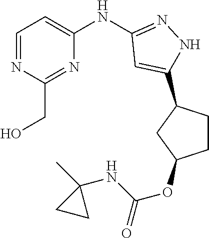 [SMILES: CC1(NC(=O)O[C@@H]2CC[C@H](c3cc(Nc4ccnc(CO)n4)n[nH]3)C2)CC1] |
| 47 | 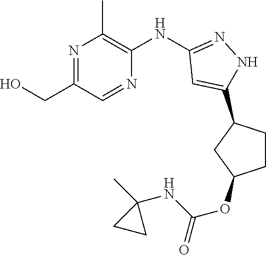 [SMILES: Cc1nc(CO)cnc1Nc1cc([C@H]2CC[C@@H](OC(=O)NC3(C)CC3)C2)[nH]n1] |
| 48 | 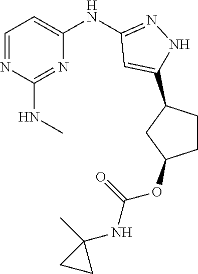 [SMILES: CNc1nccc(Nc2cc([C@H]3CC[C@@H](OC(=O)NC4(C)CC4)C3)[nH]n2)n1] |
| 49 | 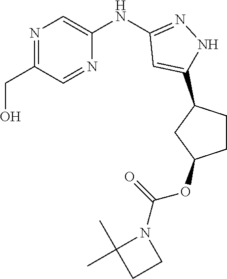 [SMILES: CC1(C)CCN1C(=O)O[C@@H]1CC[C@H](c2cc(Nc3cnc(CO)cn3)n[nH]2)C1] |
| 50 | 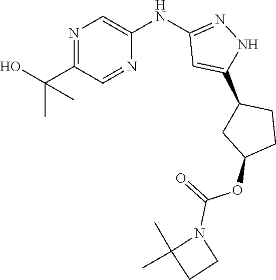 [SMILES: CC(C)(O)c1cnc(Nc2cc([C@H]3CC[C@@H](OC(=O)N4CCC4(C)C)C3)[nH]n2)cn1] |
| 51 | 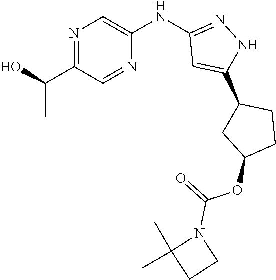 [SMILES: C[C@@H](O)c1cnc(Nc2cc([C@H]3CC[C@@H](OC(=O)N4CCC4(C)C)C3)[nH]n2)cn1] |
| | OR |
| | 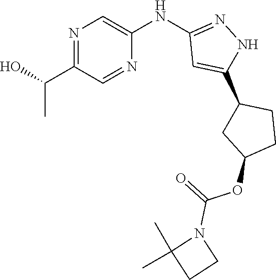 [SMILES: C[C@H](O)c1cnc(Nc2cc([C@H]3CC[C@@H](OC(=O)N4CCC4(C)C)C3)[nH]n2)cn1] |
| 52 | 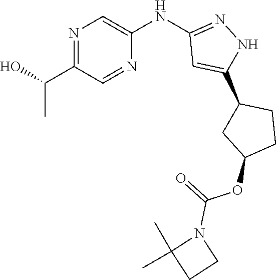 [SMILES: C[C@H](O)c1cnc(Nc2cc([C@H]3CC[C@@H](OC(=O)N4CCC4(C)C)C3)[nH]n2)cn1] |
| | OR |
| | 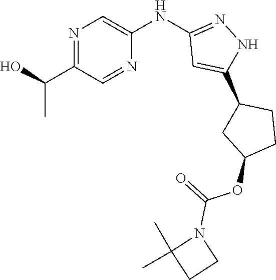 [SMILES: C[C@@H](O)c1cnc(Nc2cc([C@H]3CC[C@@H](OC(=O)N4CCC4(C)C)C3)[nH]n2)cn1] |
| 53 | 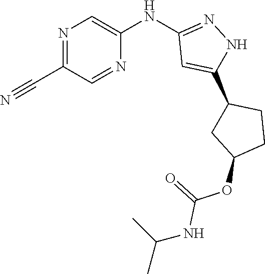 [SMILES: CC(C)NC(=O)O[C@@H]1CC[C@H](c2cc(Nc3cnc(C#N)cn3)n[nH]2)C1] |
| 54 | 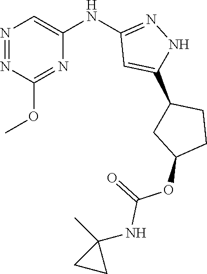 [SMILES: COc1nncc(Nc2cc([C@H]3CC[C@@H](OC(=O)NC4(C)CC4)C3)[nH]n2)n1] |
| 55 | 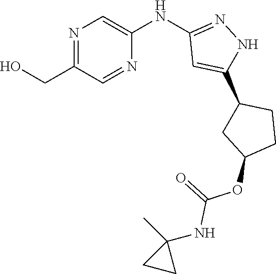 [SMILES: CC1(NC(=O)O[C@@H]2CC[C@H](c3cc(Nc4cnc(CO)cn4)n[nH]3)C2)CC1] |
| 56 | 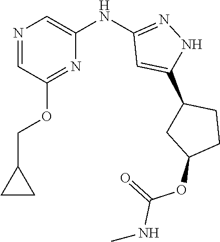 [SMILES: CNC(=O)O[C@@H]1CC[C@H](c2cc(Nc3cncc(OCC4CC4)n3)n[nH]2)C1] |
| 57 | 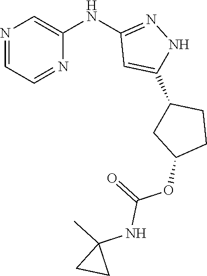 [SMILES: CC1(NC(=O)O[C@H]2CC[C@@H](c3cc(Nc4cnccn4)n[nH]3)C2)CC1] |
| 58 | 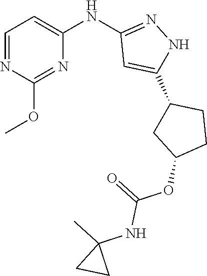 [SMILES: COc1nccc(Nc2cc([C@@H]3CC[C@H](OC(=O)NC4(C)CC4)C3)[nH]n2)n1] |
| 59 | 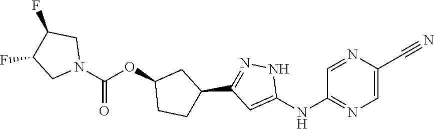 [SMILES: N#Cc1cnc(Nc2cc([C@H]3CC[C@@H](OC(=O)N4C[C@H](F)[C@@H](F)C4)C3)n[nH]2)cn1] |
| 60 | 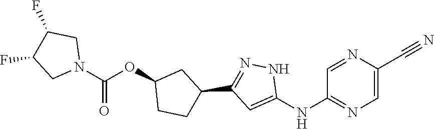 [SMILES: N#Cc1cnc(Nc2cc([C@H]3CC[C@@H](OC(=O)N4C[C@@H](F)[C@@H](F)C4)C3)n[nH]2)cn1] |
| 61 | 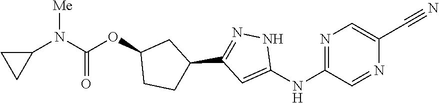 [SMILES: CN(C(=O)O[C@@H]1CC[C@H](c2cc(Nc3cnc(C#N)cn3)[nH]n2)C1)C1CC1] |
| 62 | 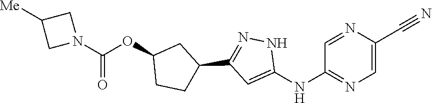 [SMILES: CC1CN(C(=O)O[C@@H]2CC[C@H](c3cc(Nc4cnc(C#N)cn4)[nH]n3)C2)C1] |
| 63 | 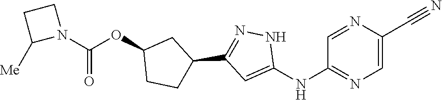 [SMILES: CC1CCN1C(=O)O[C@@H]1CC[C@H](c2cc(Nc3cnc(C#N)cn3)[nH]n2)C1] |
| 64 | 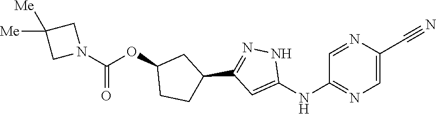 [SMILES: CC1(C)CN(C(=O)O[C@@H]2CC[C@H](c3cc(Nc4cnc(C#N)cn4)[nH]n3)C2)C1] |
| 65 | 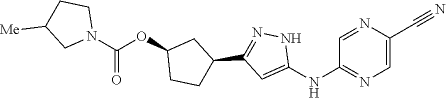 [SMILES: CC1CCN(C(=O)O[C@@H]2CC[C@H](c3cc(Nc4cnc(C#N)cn4)[nH]n3)C2)C1] |
| 66 | 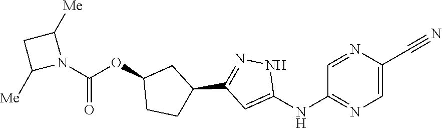 [SMILES: CC1CC(C)N1C(=O)O[C@@H]1CC[C@H](c2cc(Nc3cnc(C#N)cn3)[nH]n2)C1] |
| 67 | 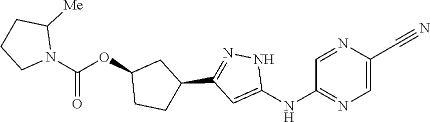 [SMILES: CC1CCCN1C(=O)O[C@@H]1CC[C@H](c2cc(Nc3cnc(C#N)cn3)[nH]n2)C1] |
| 68 | 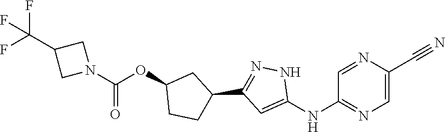 [SMILES: N#Cc1cnc(Nc2cc([C@H]3CC[C@@H](OC(=O)N4CC(C(F)(F)F)C4)C3)n[nH]2)cn1] |
| 69 | 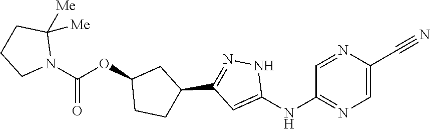 [SMILES: CC1(C)CCCN1C(=O)O[C@@H]1CC[C@H](c2cc(Nc3cnc(C#N)cn3)[nH]n2)C1] |
| 70 | 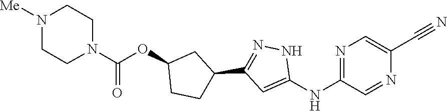 [SMILES: CN1CCN(C(=O)O[C@@H]2CC[C@H](c3cc(Nc4cnc(C#N)cn4)[nH]n3)C2)CC1] |
| 71 | 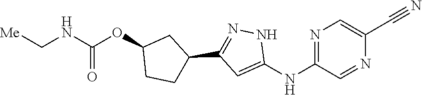 [SMILES: CCNC(=O)O[C@@H]1CC[C@H](c2cc(Nc3cnc(C#N)cn3)[nH]n2)C1] |
| 72 | 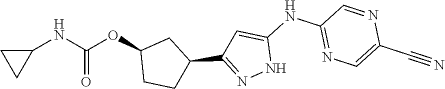 [SMILES: N#Cc1cnc(Nc2cc([C@H]3CC[C@@H](OC(=O)NC4CC4)C3)n[nH]2)cn1] |
| 73 | 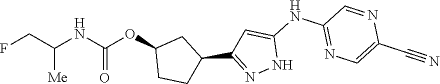 [SMILES: CC(CF)NC(=O)O[C@@H]1CC[C@H](c2cc(Nc3cnc(C#N)cn3)[nH]n2)C1] |
| 74 | 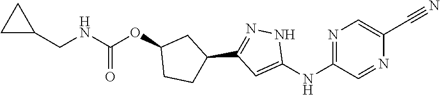 [SMILES: N#Cc1cnc(Nc2cc([C@H]3CC[C@@H](OC(=O)NCC4CC4)C3)n[nH]2)cn1] |
| 75 | 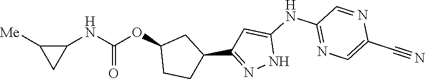 [SMILES: CC1CC1NC(=O)O[C@@H]1CC[C@H](c2cc(Nc3cnc(C#N)cn3)[nH]n2)C1] |
| 76 | 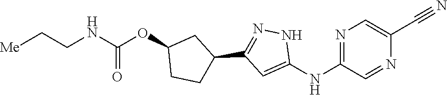 [SMILES: CCCNC(=O)O[C@@H]1CC[C@H](c2cc(Nc3cnc(C#N)cn3)[nH]n2)C1] |
| 77 | 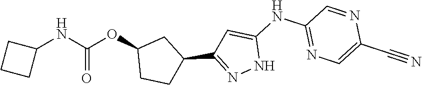 [SMILES: N#Cc1cnc(Nc2cc([C@H]3CC[C@@H](OC(=O)NC4CCC4)C3)n[nH]2)cn1] |
| 78 | 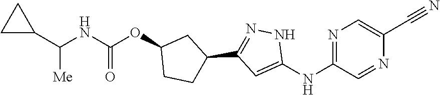 [SMILES: CC(NC(=O)O[C@@H]1CC[C@H](c2cc(Nc3cnc(C#N)cn3)[nH]n2)C1)C1CC1] |
| 79 | 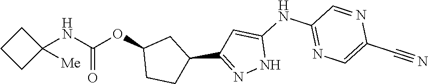 [SMILES: CC1(NC(=O)O[C@@H]2CC[C@H](c3cc(Nc4cnc(C#N)cn4)[nH]n3)C2)CCC1] |
| 80 | 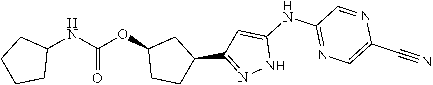 [SMILES: N#Cc1cnc(Nc2cc([C@H]3CC[C@@H](OC(=O)NC4CCCC4)C3)n[nH]2)cn1] |
| 81 | 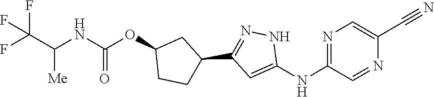 [SMILES: CC(NC(=O)O[C@@H]1CC[C@H](c2cc(Nc3cnc(C#N)cn3)[nH]n2)C1)C(F)(F)F] |
| 82 | 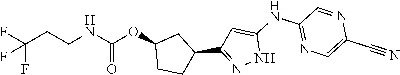 [SMILES: N#Cc1cnc(Nc2cc([C@H]3CC[C@@H](OC(=O)NCCC(F)(F)F)C3)n[nH]2)cn1] |
| 83 | 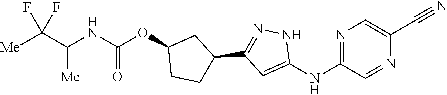 [SMILES: CC(NC(=O)O[C@@H]1CC[C@H](c2cc(Nc3cnc(C#N)cn3)[nH]n2)C1)C(C)(F)F] |
| 84 | 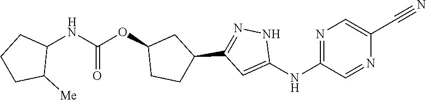 [SMILES: CC1CCCC1NC(=O)O[C@@H]1CC[C@H](c2cc(Nc3cnc(C#N)cn3)[nH]n2)C1] |
| 85 | 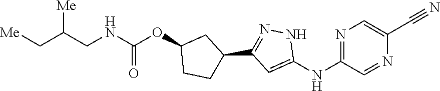 [SMILES: CCC(C)CNC(=O)O[C@@H]1CC[C@H](c2cc(Nc3cnc(C#N)cn3)[nH]n2)C1] |
| 86 | 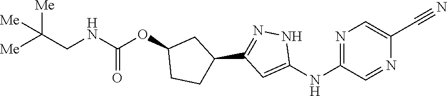 [SMILES: CC(C)(C)CNC(=O)O[C@@H]1CC[C@H](c2cc(Nc3cnc(C#N)cn3)[nH]n2)C1] |
| 87 | 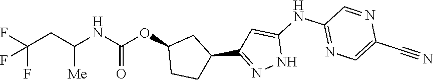 [SMILES: CC(CC(F)(F)F)NC(=O)O[C@@H]1CC[C@H](c2cc(Nc3cnc(C#N)cn3)[nH]n2)C1] |
| 88 | 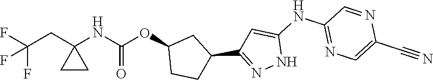 [SMILES: N#Cc1cnc(Nc2cc([C@H]3CC[C@@H](OC(=O)NC4(CC(F)(F)F)CC4)C3)n[nH]2)cn1] |
| 89 | 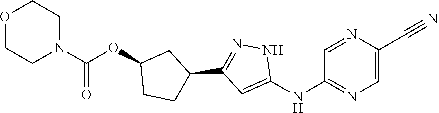 [SMILES: N#Cc1cnc(Nc2cc([C@H]3CC[C@@H](OC(=O)N4CCOCC4)C3)n[nH]2)cn1] |
| 90 | 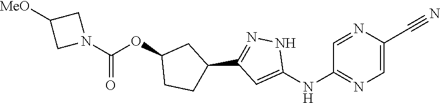 [SMILES: COC1CN(C(=O)O[C@@H]2CC[C@H](c3cc(Nc4cnc(C#N)cn4)[nH]n3)C2)C1] |
| 91 | 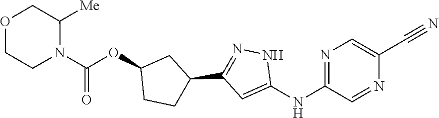 [SMILES: CC1COCCN1C(=O)O[C@@H]1CC[C@H](c2cc(Nc3cnc(C#N)cn3)[nH]n2)C1] |
| 92 | 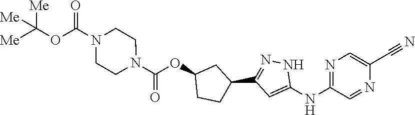 [SMILES: CC(C)(C)OC(=O)N1CCN(C(=O)O[C@@H]2CC[C@H](c3cc(Nc4cnc(C#N)cn4)[nH]n3)C2)CC1] |
| 93 | 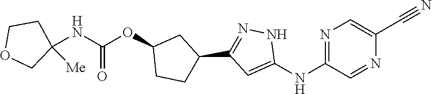 [SMILES: CC1(NC(=O)O[C@@H]2CC[C@H](c3cc(Nc4cnc(C#N)cn4)[nH]n3)C2)CCOC1] |
| 94 | 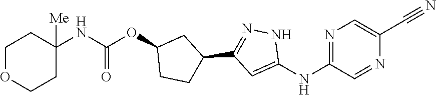 [SMILES: CC1(NC(=O)O[C@@H]2CC[C@H](c3cc(Nc4cnc(C#N)cn4)[nH]n3)C2)CCOCC1] |
| 95 | 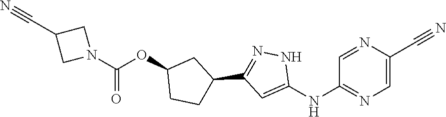 [SMILES: N#Cc1cnc(Nc2cc([C@H]3CC[C@@H](OC(=O)N4CC(C#N)C4)C3)n[nH]2)cn1] |
| 96 | 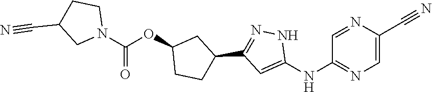 [SMILES: N#Cc1cnc(Nc2cc([C@H]3CC[C@@H](OC(=O)N4CCC(C#N)C4)C3)n[nH]2)cn1] |
| 97 | 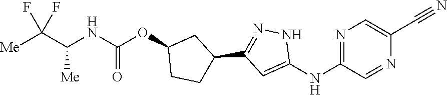 [SMILES: C[C@@H](NC(=O)O[C@@H]1CC[C@H](c2cc(Nc3cnc(C#N)cn3)[nH]n2)C1)C(C)(F)F] |
| 98 | 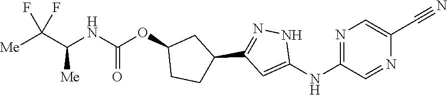 [SMILES: C[C@H](NC(=O)O[C@@H]1CC[C@H](c2cc(Nc3cnc(C#N)cn3)[nH]n2)C1)C(C)(F)F] |
| 100 | 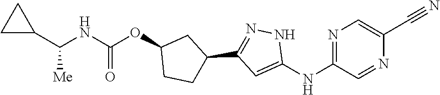 [SMILES: C[C@@H](NC(=O)O[C@@H]1CC[C@H](c2cc(Nc3cnc(C#N)cn3)[nH]n2)C1)C1CC1] |
| 101 | 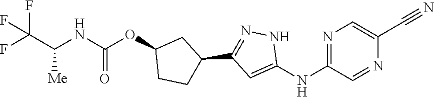 [SMILES: C[C@@H](NC(=O)O[C@@H]1CC[C@H](c2cc(Nc3cnc(C#N)cn3)[nH]n2)C1)C(F)(F)F] |
| 102 | 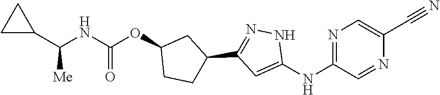 [SMILES: C[C@H](NC(=O)O[C@@H]1CC[C@H](c2cc(Nc3cnc(C#N)cn3)[nH]n2)C1)C1CC1] |
| 103 | 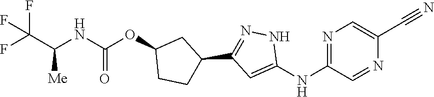 [SMILES: C[C@H](NC(=O)O[C@@H]1CC[C@H](c2cc(Nc3cnc(C#N)cn3)[nH]n2)C1)C(F)(F)F] |
| 104 | 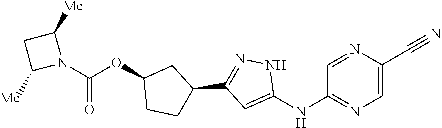 [SMILES: C[C@@H]1C[C@@H](C)N1C(=O)O[C@@H]1CC[C@H](c2cc(Nc3cnc(C#N)cn3)[nH]n2)C1] |
| 105 | 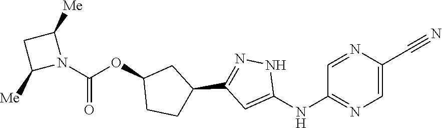 [SMILES: C[C@@H]1C[C@H](C)N1C(=O)O[C@@H]1CC[C@H](c2cc(Nc3cnc(C#N)cn3)[nH]n2)C1] |
| 106 | 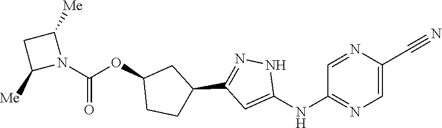 [SMILES: C[C@H]1C[C@H](C)N1C(=O)O[C@@H]1CC[C@H](c2cc(Nc3cnc(C#N)cn3)[nH]n2)C1] |
| 107 | 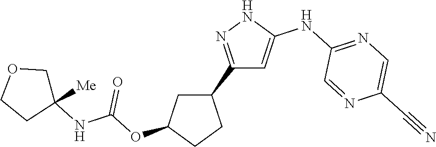 [SMILES: C[C@@]1(NC(=O)O[C@@H]2CC[C@H](c3cc(Nc4cnc(C#N)cn4)[nH]n3)C2)CCOC1] |
| 108 | 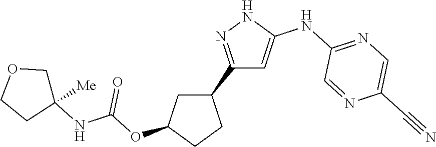 [SMILES: C[C@]1(NC(=O)O[C@@H]2CC[C@H](c3cc(Nc4cnc(C#N)cn4)[nH]n3)C2)CCOC1] |

The term “pharmaceutically-acceptable salt” refers to a pharmaceutical salt that is, within the scope of sound medical judgment, suitable for use in contact with the tissues of humans and lower animals without undue toxicity, irritation, and allergic response, and is commensurate with a reasonable benefit/risk ratio. Pharmaceutically-acceptable salts are well known in the art. For example, S. M. Berge et al. describes pharmacologically acceptable salts in J. Pharm. Sci., 1977, 66, 1-19.

Included in the present teachings are pharmaceutically acceptable salts of the compounds disclosed herein. Compounds having basic groups can form pharmaceutically acceptable salts with pharmaceutically acceptable acid(s). Suitable pharmaceutically acceptable acid addition salts of the compounds described herein include salts of inorganic acids (such as hydrochloric, hydrobromic, phosphoric, metaphosphoric, nitric, and sulfuric acids) and of organic acids (such as acetic, benzenesulfonic, benzoic, ethanesulfonic, methanesulfonic, and succinic acids). Compounds of the present teachings with acidic groups such as carboxylic acids can form pharmaceutically acceptable salts with pharmaceutically acceptable base(s). Suitable pharmaceutically acceptable basic salts include ammonium salts, alkali metal salts (such as sodium and potassium salts) and alkaline earth metal salts (such as magnesium and calcium salts).

Compounds having one or more chiral centers can exist in various stereoisomeric forms, i.e., each chiral center can have an R or S configuration, or can be a mixture of both. Stereoisomers are compounds that differ only in their spatial arrangement. Stereoisomers include all diastereomeric and enantiomeric forms of a compound. Enantiomers are stereoisomers that are mirror images of each other. Diastereomers are stereoisomers having two or more chiral centers that are not identical and are not mirror images of each other.

When the stereochemical configuration at a chiral center in a compound having one or more chiral centers is depicted by its chemical name (e.g., where the configuration is indicated in the chemical name by “R” or “S”) or structure (e.g., the configuration is indicated by “wedge” bonds), the enrichment of the indicated configuration relative to the opposite configuration is greater than 50%, 60%, 70%, 80%, 90%, 99% or 99.9% (except when the designation “rac” or “racemate accompanies the structure or name, as explained in the following two paragraphs). “Enrichment of the indicated configuration relative to the opposite configuration” is a mole percent and is determined by dividing the number of compounds with the indicated stereochemical configuration at the chiral center(s) by the total number of all of the compounds with the same or opposite stereochemical configuration in a mixture.

When the stereochemical configuration at a chiral center in a compound is depicted by chemical name (e.g., where the configuration is indicated in the name by “R” or “S”) or structure (e.g., the configuration is indicated by “wedge” bonds) and the designation “rac” or “racemate” accompanies the structure or is designated in the chemical name, a racemic mixture is intended.

When two stereoisomers are depicted by their chemical names or structures, and the names or structures are connected by an “or”, one or the other of the two stereoisomers is intended, but not both.

When a disclosed compound having a chiral center is depicted by a structure without showing a configuration at that chiral center, the structure is meant to encompass the compound with the S configuration at that chiral center, the compound with the R configuration at that chiral center, or the compound with a mixture of the R and S configuration at that chiral center. When a disclosed compound having a chiral center is depicted by its chemical name without indicating a configuration at that chiral center with “S” or “R”, the name is meant to encompass the compound with the S configuration at that chiral center, the compound with the R configuration at that chiral center or the compound with a mixture of the R and S configuration at that chiral center.

A racemic mixture means a mixture of 50% of one enantiomer and 50% of its corresponding enantiomer. The present teachings encompass all enantiomerically-pure, enantiomerically-enriched, diastereomerically pure, diastereomerically enriched, and racemic mixtures, and diastereomeric mixtures of the compounds disclosed herein.

Enantiomeric and diastereomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

“Peak 1” in the Experimental section refers to an intended reaction product compound obtained from a chromatography separation/purification that elutes earlier than a second intended reaction product compound from the same preceding reaction. The second intended product compound is referred to as “peak 2”.

When a disclosed compound is designated by a name or structure that indicates a single enantiomer, unless indicated otherwise, the compound is at least 60%, 70%, 80%, 90%, 99% or 99.9% optically pure (also referred to as “enantiomerically pure”). Optical purity is the weight in the mixture of the named or depicted enantiomer divided by the total weight in the mixture of both enantiomers.

When the stereochemistry of a disclosed compound is named or depicted by structure, and the named or depicted structure encompasses more than one stereoisomer (e.g., as in a diastereomeric pair), it is to be understood that, unless otherwise indicated, one of the encompassed stereoisomers or any mixture of the encompassed stereoisomers are included. It is to be further understood that the stereoisomeric purity of the named or depicted stereoisomers at least 60%, 70%, 80%, 90%, 99% or 99.9% by weight. The stereoisomeric purity in this case is determined by dividing the total weight in the mixture of the stereoisomers encompassed by the name or structure by the total weight in the mixture of all of the stereoisomers.

In the compounds of the disclosure, any position specifically designated as “D” or “deuterium” is understood to have deuterium enrichment at 50, 80, 90, 95, 98 or 99%. “Deuterium enrichment” is a mole percent and is determined by dividing the number of compounds with deuterium at the indicated position by the total number of all of the compounds. When a position is designated as “I” or “hydrogen”, the position has hydrogen at its natural abundance. When a position is silent as to whether hydrogen or deuterium is present, the position has hydrogen at its natural abundance. One specific alternative embodiment is directed to a compound of the disclosure having deuterium enrichment of at least 5, 10, 25, 50, 80, 90, 95, 98 or 99% at one or more positions not specifically designated as “D” or “deuterium”.

As used herein, many moieties (e.g., alkyl, alkoxy, cycloalkyl or heterocyclyl) are referred to as being either “substituted” or “optionally substituted”. When a moiety is modified by one of these terms, unless otherwise rioted, it denotes that any portion of the moiety that is known to one skilled in the art as being available for substitution can be substituted, which includes one or more substituents. Where if more than one substituent is present, then each substituent may be independently selected. Such means for substitution are well-known in the art and/or taught by the instant disclosure. The optional substituents can be any substituents that are suitable to attach to the moiety.

Compounds of the disclosure are CDK2 inhibitors. As used herein, the term “selective CDK2 inhibitor” means a compound which selectively inhibits CDK2 over other CDKs and the kinome. Said another way, a selective CDK2 inhibitor has no or low activity against other CDKs and the kinome. A selective CDK2 inhibitor's inhibitory activity against CDK2 is more potent in terms of IC₅₀ value (i.e., the IC₅₀ value is subnanomolar) when compared with its inhibitory activity against other CDKs and many other kinases. Potency can be measured using known biochemical assays.

In some embodiments, the compounds of the disclosure are selective against CDK2 versus CDK1. In some such embodiments, compounds show at least 10-fold selectivity for CDK2 versus CDK1. In other embodiments, compounds show at least 20-fold selectivity for CDK2 versus CDK1. In specific embodiments, compounds show at least 30-fold selectivity for CDK2, versus CDK1. In certain embodiments, compounds show at least 40-fold selectivity for CDK2 versus CDK1. In other embodiments, compounds show at least 50-fold selectivity or CDK2 versus CDK1. For example, disclosed compounds show at least 100-fold selectivity for CDK2 versus CDK1. In some embodiments, disclosed compounds are selective against CDK2 versus CDK4 and/or CDK6. In some such embodiments, compounds show at least 10-fold selectivity for CDK2 versus CDK4 and/or CDK6. In other embodiments, disclosed compounds show at least 20-fold selectivity for CDK2 versus CDK4 and/or CDK6. In specific embodiments, compounds show at least 30-fold selectivity for CDK2 versus CDK4 and/or CDK6.

Some compounds of the disclosure have the advantage of good metabolic stability. One indicator of good metabolic stability is high microsomal stability. Hepatic metabolism is a predominant route of elimination for small molecule drugs. The clearance of compounds by hepatic metabolism can be assessed in vitro using human liver microsomes (HLMs) or human hepatocytes. Compounds are incubated with HLMs plus appropriate co-factors or human hepatocytes and compound depletion is measured to determine an in vitro intrinsic clearance (Clint). The Clint is scaled to total body clearance (CL), and a hepatic extraction ratio (ER) is determined by dividing CL to standard human hepatic blood flow. Compounds that have a low hepatic extraction ratio are considered to have good metabolic stability. In some embodiments, a compound of the disclosure has a calculated ER of <0.3, <0.4, <0.5, <0.6.

### Pharmaceutical Compositions

Pharmaceutical compositions of the disclosure (also referred to herein as the “disclosed pharmaceutical compositions”) comprise one or more pharmaceutically acceptable carrier(s) or diluent(s) and a compound of the disclosure (e.g., a compound of Formula (I) or Formula (I-1)), or a pharmaceutically acceptable salt thereof.

“Pharmaceutically acceptable carrier” and “pharmaceutically acceptable diluent” refer to a substance that aids the formulation and/or administration of an active agent to and/or absorption by a subject and can be included in the pharmaceutical compositions of the disclosure without causing a significant adverse toxicological effect on the subject. Non-limiting examples of pharmaceutically acceptable carriers and/or diluents include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, hydroxymethylcellulose, fatty acid esters, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with or interfere with the activity of the compounds provided herein. One of ordinary skill in the art will recognize that other pharmaceutical excipients are suitable for use with disclosed compounds or pharmaceutically acceptable salts thereof.

The pharmaceutical compositions of the disclosure optionally include one or more pharmaceutically acceptable carriers and/or diluents therefor, such as lactose, starch, cellulose and dextrose. Other excipients, such as flavoring agents, sweeteners, and preservatives, such as methyl, ethyl, propyl and butyl parabens, can also be included. More complete listings of suitable excipients can be found in the Handbook of Pharmaceutical Excipients (5^{th} Ed., Pharmaceutical Press (2005)). A person skilled in the art would know how to prepare formulations suitable for various types of administration routes. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999. The carriers, diluents and/or excipients are “acceptable” in the sense of being compatible with the other ingredients of the pharmaceutical composition and not deleterious to the recipient thereof.

### Methods of Treatment

The compounds disclosed herein inhibit CDK2 and therefore are useful for treating diseases for which CDK2 is dysregulated, such as cancer. The present disclosure provides a method of inhibiting CDK2 in a subject in need thereof, comprising administering to the subject an effective amount of a compound disclosed herein, a pharmaceutically acceptable salt thereof or a pharmaceutical composition disclosed herein.

In some embodiments, the disclosure provides a method of treating a disease or disorder associated with CDMK2 in a patient, comprising administering to the patient a therapeutical effective amount of a compound of Formula (I), Formula (I-1), or any of the formulas as described herein, or a pharmaceutically acceptable salt thereof. In some embodiments, the disease or disorder associated with CDK2 is associated with an amplificatory of the cyclin E1 (CCNE1) gene and/or overexpression of CCNE1. In some embodiments, the disease or disorder is cancer.

Subjects “in need of inhibiting CDK2” are those having a disease for which a beneficial therapeutic effect can be achieved by inhibiting CDK2, e.g., a slowing in disease progression, alleviation of one or more symptoms associated with the disease or increasing the longevity of the subject in view of the disease.

In some embodiments, the disclosure provides a method of treating a disease/condition/or cancer associated with or modulated by CDK2, wherein the inhibition of CDK2 is of therapeutic benefit, including but not limited to the treatment of cancer in a subject in need thereof. The method comprises administering to the subject an effective amount of a compound disclosed herein, a pharmaceutically acceptable salt thereof, or pharmaceutical composition disclosed herein.

In another embodiment, the disclosure provides a method of treating a subject with cancer, comprising administering to the subject an effective amount of a compound disclosed herein, a pharmaceutically acceptable salt thereof, or a pharmaceutical composition disclosed herein. In another embodiment, the cancer is characterized by amplification and/or overexpression of CCNE1 or CCNE2.

Accordingly, in some embodiments of the methods, the subject or patient has been previously determined to have an amplification of the cyclin E1 (CCNE1) gene and/or an expression level of CCNE1 in a biological sample obtained from the subject or patient that is higher than a control expression level of CCNE1.

In another embodiment, the disclosure provides a method for inhibiting growth of tumor (e.g., cancer) cells in vitro. The method includes contacting the tumor (e.g. cancer) cells in vitro with a compound of Formula (I) or Formula (I-1) or a pharmaceutically acceptable salt thereof. In another embodiment, the present disclosure provides a method for inhibiting growth of turner (e.g., cancer) cells with CCNE1 amplification and/or overexpression in a subject or a patient. The method includes administering to the subject or patient in need thereof a therapeutically effective amount of a compound of Formula (I) or Formula (I-1), or a pharmaceutically acceptable salt thereof.

In another embodiment, the disclosure provides a method of treating a subject with cancer, comprising administering to the subject an effective amount of a compound disclosed herein, a pharmaceutically acceptable salt thereof, or a pharmaceutical composition disclosed herein in conjunction with other agents or standard cancer treatments, as described below.

As used herein “cancer” refers to any malignant and/or invasive growth or tumor caused by abnormal cell growth. Cancer includes solid tumors named for the type of cells that form them, cancer of blood, bone marrow, or the lymphatic system. Examples of solid tumors include sarcomas and carcinomas. Cancers of the blood include, but are not limited to, leukemia, lymphoma and myeloma. Cancer also includes primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of a different type from the latter one. In some such embodiments, the cancer is characterized by amplification and/or overexpression of CCNE1 and/or CCNE2.

Cancers to be treated according to the disclosed methods include breast cancer, ovarian cancer, bladder cancer, uterine cancer (e.g., uterine carcinosarcoma), prostate cancer, lung cancer (including NSCLC, SCLC, squamous cell carcinoma (e.g., lung squamous cell carcinoma (LUSC)), or adenocarcinoma (e.g., lung adenocarcinoma (LUAD))), esophageal cancer, head and neck cancer, colorectal cancer (e.g., colon cancer, colorectal adenocarcinoma (COADREAD)), kidney cancer (including RCC), liver cancer (including HCC), pancreatic cancer, stomach (i.e., gastric) cancer, urothelial cancer, brain cancers, mesothelioma (MESO), skin cancer (e.g., melanoma), sarcoma, or thyroid cancer, including metastasis (in particular brain metastasis) of all cancers listed. In some embodiments, the cancer is characterized by at overexpression and/or amplification of CCNE1 and/or CCNE2 described herein. In some embodiments of the methods provided herein, the subject is identified as having a cancer characterized by amplification and/or overexpression of CCNE1 and/or CCNF2.

In further embodiments of the methods provided herein, the cancer is breast cancer, ovarian cancer, bladder cancer, uterine cancer, prostate cancer, lung cancer, esophageal cancer, liver cancer, pancreatic cancer or stomach cancer. In some such embodiments, the cancer is characterized by amplification and/or overexpression of CCNE1 and/or CCNE2.

In further embodiments, of the methods provided herein, the cancer is selected from the group consisting of ovarian cancer endometrial cancer, gastric cancer, esophageal cancer, triple negative breast cancer, and lung adenosarcoma. In some embodiments, the cancer is characterized by CCNE1 overexpression and/or amplification. In some embodiments, the cancer has progressed despite platinum treatment.

In some embodiments, the cancer is platinum-resistant and/or platinum-refractory. In some embodiments, the cancer has progressed despite platinum treatment.

In some embodiments, the disease or disorder associated with CDK2 is a adenocarcinoma carcinoma, or cystadenocarcinoma.

In other embodiments, the cancer is breast cancer, including, e.g., ER-positive/HR-positive, HER2-negative breast cancer; ER-positive/I R-positive, HER2-positive breast cancer: triple negative breast cancer (TNBC); or inflammatory breast cancer. In some embodiments, the breast cancer is chemotherapy or radiotherapy resistant breast cancer, endocrine resistant breast cancer, trastuzumab resistant breast cancer, or breast cancer demonstrating primary or acquired resistance to CDK4/CDK6 inhibition. In some embodiments, the breast cancer is advanced or metastatic breast cancer, in some embodiments of each of the foregoing, the breast cancer is characterized by amplification and/or overexpression of CCNEN1 and/or CCNE2.

In some embodiments, the cancer is HR-positive breast cancer. In some embodiments, the breast cancer is ER-positive breast cancer. In some embodiments, the breast cancer is HR-positive, HER-negative breast cancer, in some embodiments, the breast cancer is ER-positive, HER2-negative breast cancer. In some embodiments, the breast cancer is responsive to treatment with a CDK4/6 inhibitor In some embodiments, the breast cancer is resistant to treatment with a CDK4/6 inhibitor. In some embodiments, the breast cancer has progressed despite treatment with a CDK4/6 inhibitor. In some embodiments, the CDK4/6 inhibitor is palbociclib. In some embodiments, the breast cancer has progressed despite first treatment with palbociclib and/or fulvestrant and second treatment with abemaciclib and/or fulvestrant. In some embodiments, the method further comprises administering an effective amount of a CDK4/6 inhibitor. In some embodiments, the CDK4/6 inhibitor is selected from palbociclib and ribociclib, or a combination thereof. In some embodiments, the CDK4/6 inhibitor is ribociclib. In some embodiments, the breast cancer has CCNE amplification aid/or overexpression.

In some embodiments, the breast cancer is triple negative breast cancer.

In some embodiments, the cancer is ovarian cancer. In some such embodiments, the cancer is ovarian cancer characterized b amplification and/or overexpression of CCNE1 and/or CCNEE2. In some such embodiments, the cancer is (a) ovarian cancer; (b) characterized by amplification and/or overexpression of cyclin E1 (CCNE1) or cyclin E2 (CCNE2) or (c) both (a) and (b). In some embodiments, the cancer is ovarian cancer.

In some embodiments, the compound of the disclosure is adminstered as first line therapy. In other embodiments, the compound of the disclosure is administered as second (or later) line therapy. In some embodiments, the compound of the disclosure is administered as second (or later) line therapy following treatment with an endocrine therapeutic agent and/or a CDK4/CDK6 inhibitor. In some embodiments, the compound of the disclosure is administered as second (or later) line therapy following, treatment with an endocrine therapeutic agent e.g., an aromatase inhibitor, a SERM or a SERD. In some embodiments, the compound of the disclosure is administered as second (or later) line therapy following treatment with a CDK4/CDK6 inhibitor. In some embodiments, the compound of the disclosure is adminstered as second (or later) line therapy following treatment with one or more chemotherapy regimens, e.g., including taxanes or platinum agents. In some embodiments, the compound of the disclosure is administered as second (or later) line therapy following treatment with HER2 targeted agents, e.g., trastuzumab.

In some embodiments, the disease or disorder associated with CDK2 is N-myc amplified neuroblastoma cells (see Molenaar, et al., Proc Natl Acad Sci USA 106(31): 12968-12973) K-Ras mutant lung cancers (see Hu, S., et al., Mol Cancer Ther, 2015. 14(11): 2576-85, and cancers with FBW7 mutation and CCNE1 overexpression. (see Takada, et al., Cancer Res, 2017. 77(18): 4881-4893).

In some embodiments, the compounds of the present disclosure can be used to treat sickle cell disease and sickle cell anemia.

Examples of cancers that are treatable using the compounds of the present disclosure include, but are not limited to, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymnphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor brain stem glioma, pituitary adenoma. Kaposi's sarcoma, epidermoid cancer, squanmous cell cancer, T-cell lymphoma, environmentally induced cancers including those induced by asbestos, and combinations of said cancers. The compounds of the present disclosure are also useful for the treatment of metastatic cancers.

In some embodiments, cancers treatable with compounds of the present disclosure include, but are not limited to, melanoma (e.g., metastatic malignant melanoma, BRAF and HSP90 inhibition-resistant melanoma, skin cutaneous melanoma (SKCM)) renal cancer (e.g., clear cell carcinoma), prostate cancer (e.g., hormone retractors' prostate adenocarcinoma), breast cancer, colon cancer, lung cancer (e.g., non-small cell lung cancer mad small cell lung cancer), squamous cell head and neck cancer (e.g., head and neck squamous cell carcinoma (NHSC)), urothelial cancer (e g bladder) and cancers with high microsatellite instability (MSIhigh). Additionally, the disclosure includes refractory or recurrent malignancies whose growth may be inhibited using the compounds of the disclosure.

In some embodiments, cancers that are treatable using the compounds of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.) hematological cancers (e.g., lymphoma, leukemia such as acute lymphoblastic leukemia (ALL), acute myelogenous leukemia (AML), chronic lymphocitic leukemia (CLL), chronic myelogenous leukemia (CML), DLBCL, mantle cell lymphoma, Non-Hodgkin lymphoma (including follicular lymphoma, including relapsed or refractory NHL and recurrent follicular), Hodgkin lymphoma or multiple myeloma) and combinations of said cancers.

In some embodiments, cancers that are treatable using the compounds of the present disclosure include, but are not limited to, cholangiocarcinoma, bile duct cancer, triple negative breast cancer, rhabdomyosarcoma, small cell lung cancer, leiomyosarcoma, hepatocellular carcinoma (e.g., liver hepatocellular carcinoma (LIHC)), Ewing's sarcoma, brain cancer, bin tumor, astrocytoma, neuroblastoma, neurofibroma, basal cell carinoma, chondrosarcoma, epithelioid sarcoma, eye cancer, Fallopian tube cancer, gastrointestinal cancer, gastrointestinal stromal tumors, hairy cell leukemia, intestinal cancer, islet cell cancer, oral cancer, mouth cancer, throat cancer, laryngeal cancer, lip cancer, mesothelioma, neck cancer, nasal cavity cancer, ocular cancer, ocular melanoma, pelvic cancer, rectal cancer, renal cell carcinoma, salivary gland cancer, sinus cancer, spinal cancer, tongue cancer, tubular carcinoma, urethral cancer, and ureteral cancer.

In some embodiments, cancers treatable with compounds of the present disclosure include Genomic Identification of Significant Targets in Cancer (GISTIC) and pheochromocytoma and paraganglioma (PCPG).

In some embodiments, cancers treatable with compounds of the present disclosure include advanced/relapsed tumors; CCNE1 amplified platinum-resistant or platinum-refractory ovarian cancer; endometrial cancer (with prior platinum therapy) that has progressed following 2 or more lines of therapies; and gastric cancer (with prior platinum therapy) that has progressed following 2 or more lines of therapies; and ER+ HER2− BC that has progressed despite CDK4/6i. In some embodiments, cancers treatable with compounds of the present disclosure include Platinum-resistant or platinum-refractory CCNE1 amplified ovarian cancer; CCNE1 amplified endometrial cancer that has failed 2 or more lines of therapies; CCNE1 amplified advanced/relapsed tumors that do not belong to the other groups. ER+ HER2− BC that has progressed despite CDK4/6i; platinum-resistant or platinum-refractory CCNE1 amplified ovarian cancer; and ER+ HER2− BC that has progressed despite CDK4/6i.

In some embodiments, diseases and indications that are treatable using the compounds of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia Nera (PV), and essential thrombocytosis (ET)), myelodysplasia syndrome (MDS), T-cell acute lymphoblastic lymphoma (T-ALL) and multiple myeloma (MM).

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, hamartoma, and teratoma.

Exemplary lung cancers include non-small cell lung cancer (NSCLC), small cell lung cancer (SCLC), bronchogemc carcinoma, squamous cell, undifferentiated small cell, undifferentiated large cell, adenocarcinoma, alveolar (bronchiolar) carcinoma, bronchial adenoma, chondromatous hamartoma, and mesothelioma. Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), and colorectal cancer.

Exemplary genitourinary tract cancers include cancers of the kidney (adenocarcinoma, Wilm's tumor [nephroblastoma]), bladder and urethra (squamous cell carcinoma, transitional cell carcinoma, adenocarcinoma), prostate (adenocarcinoma (PRAD), sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma).

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chrondroma, chondroblastoma, chondromyxofibroma, osteoid osteoma, and giant cell tumors

Exemplary nervous system cancers include cancers of the skull (osteoma, hemangioma, granuloma, xanthoma, osteitis deformans), meninges (meningioma, meningiosarcoma, gliomatosis), brain (astrocytoma, medulloblastoma, glioma, brain lower grade glioma (LOG), ependymoma, germinoma (pinealoma), glioblastoma, glioblastoma multiforme (GBM), oligodendroglioma, schwannoma, retinoblastoma, congenital tumors), and spinal cord (neurofibroma, meningioma, glioma, sarcoma), as well as neuroblastoma and Lhermitte-Duclos disease.

Exemplary gynecological cancers include cancers of the uterus (endometrial carcinoma), cervix (cervical carcinoma, cervical squamous cell carcinoma (CESC), pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, Merkel cell carcinoma, squamous cell carcinoma, Kaposi's sarcoma, moles dysplastic nevi, lipoma, angioma, dermatofibroma, and keloids. In some embodiments, diseases and indications that are treatable using the compounds of the present disclosure include, but are not limited to, sickle cell disease (e.g., sickle cell anemia), triple-negative breast cancer (TNBC), myelodysplastic syndromes, testicular cancer, bile duct cancer, esophageal cancer, and urothelial carcinoma.

### Combinations

Compounds of the disclosure may be administered as single agents or may be administered in combination with other anti-cancer therapeutic agents, in particular standard of care agents appropriate for the particular cancer.

The term “additional anticancer therapeutic agent” as used herein means any one or more therapeutic agent, other than a compound of the disclosure, that is or can be used in the treatment of cancer. In some embodiments, such additional anticancer therapeutic agents include compounds derived from the following classes, mitotic inhibitors, alkylating agents, antimetabolites, antitumor antibiotics, anti-angiogenesis agents, topoisomerase I and II inhibitors, plant alkaloids, hormonal agents and antagonists, growth factor inhibitors, radiation, signal transduction inhibitors, such as inhibitors of protein tyrosine kinases and/or serine/threonine kinases, cell cycle inhibitors, biological response modifiers, enzyme inhibitors, antisense oligonucleotides or oligonucleotide derivatives, cytotoxics, immuno-oncology agents, and the like.

In some embodiments, the additional anticancer agent is an endocrine agent, such as an aromatase inhibitor, a SERD or a SERM.

In some embodiments, the additional anticancer agent is a PIK3CA inhibitor including, but not limited to, alpelisib (PIQRAY), BEBT-908, BPI-21668, buparlisib, inavolisib, TQB-3525, RLY-2608, miransertib, MEN-1611, LOXO-783, HS-10352, HH-CYH33, gedatolisib, and fimepinostat.

In some embodiments, the additional anticancer agent is an antibody-drug conjugates including, but not limited to, Trastuzumab deruxtecan (Enhertu), Trastuzumab duocarmazine, Trastuzumab emtansine (Kadcyla), Upifitamab rilsodotin, mirvetuximab soravtansine, Tisotumab vedotin (Tivdak), Praluzatamab ravtansine, Sacituzumab govitecan or Sacituzumab Govitecan-hziy (Trodelvy), Datopotamab deruxtecan, Ladiratuzumab vedotin, Patritumab deruxtecan, STRO-002, MORab-202, DS-6000, Anetumab, avtansine, XMT-2056, Disitamab Vedotin (RC48-ADC, Aidexi).

In some embodiments, the additional anticancer agent is a PLK1 inhibitor including, but not limited to onvansertib, B12536, B16727, GSK461364A, TAK960, rigosertib.

In some embodiments, the additional anticancer agent is Estrogen PROTAC (ARV-471, H3B-5942).

In other embodiments, a compound of the disclosure may be administered in combination with a standard of care agent. In some embodiments, a compound of the disclosure may be administered in combination with endocrine therapy, e.g., agents such as letrozole, fulvestrant, tamoxifen, exemestane, or anastrozole. In some embodiments, a compound of the disclosure may be adminstered in combination with a chemotherapeutic agent, e.g., docetaxel, paclitaxel, cisplatin, carboplatin, capecitabine, gemcitabine, vinorelbine, or liposomal doxorubicin. In other embodiments, a compound of the invention may be administered in combination with an anti-HER2 agent, e.g., trastuzumab or pertuzumab.

In some embodiments, a compound of the disclosure (for example, a compound of Formula (I) or Formula (I-1) or a pharmaceutically acceptable salt thereof) may be administered in combination with an effective amount of carboplatin, ribociclib, fulvestrant, or a combination thereof

In some embodiments, the additional anticancer agent is an anti-angiogenesis agent, including for example VEGF inhibitors. V EGFR inhibitors, TIE-2 inhibitors, PDGFR inhibitors, angiopoetin inhibitors, PKCb inhibitors, COX-2 (cyclooxygenase 11) inhibitors, integrins (alpha-v/beta-3), MMP-2 (matrix-metalloproteinase 2) inhibitors, and MMP-9 (matrix-metalloproteinase 9) inhibitors. Preferred anti-angiogenesis agents include sunitinib (Sutent™), bevacizumab (Avastin™), axitinib (AG 13736), SU 14813 (Pfizer), and AG 13958 (Pfizer). Additional anti-angiogenesis agents include vatalanib (CGP 79787), Sorafenib (Nexavar™), pegaptanib octasodium (Macugen™), vandetanib (Zactima™), PF-0337210 (Pfizer), SU 14843 (Pfizer), AZD 2171 (AstraZeneca), ranibizumab (Lucentis™), Neovastat™ (AE 941), tetrathiomolybdata (Coprexa™), AMG 706 (Amgen), VEGF Trap (AVE 0005), CEP 7055 (Sanofi-Aventis), XL 880 (Exelixis), telatinib (BAY 57-9352), and CP-868,596 (Pfizer). Other anti-angiogenesis agents include enzastaurin (LY 317615), midostaurin (CGP 41251), perifosine (KRX 0401), teprenone (Selbex™) and UCN 01 (Kyowa Hakko). Other examples of anti-angiogenesis agents include celecoxib (Celebrex™), parecoxib (Dynastat™), deracoxib (SC 59046), lumiracoxib (Preige™), valdecoxib (Bextra™), rofecoxib (Vioxx™), iguratimod (Careram™), IP 751 (Invedus), SC-58125 (Pharmacia) and etoricoxib (Arcoxia™). Yet further anti-angiogenesis agents include exisulind (Aptosyn™), salsalate (Amigesic™), diflunisal (Dolobid™), ibuprofen (Motrin™), ketoprofen (Orudis™), nabumetone (Relafen™), piroxicam (Feldene™), naproxen (Aleve™, Naprosyn™), diclofenac (Voltaren™), indomethacin (Indocin™), sulindac (Clinoril™), tolmetin (Tolectin™), etodolac (Lodine™), ketorolac (Toradol™), and oxaprozin (Daypro™). Yet further anti-angiogenesis agents include ABT 510 (Abbott), apratastat (TMI 005), AZD 8955 (AstraZeneca), incyclinide (Metastat™), and PCK 3145 (Procyon).

Yet further anti-angiogenesis agents (including VEGFR/PDGFR inhibitors) include, but are not limited to, ponatinib (Iclusig), BT1718, anlotinib, lenvatinib (Lenvima), tivozanib (Fotivda), dovitinib, brolucizumab (Beovu), aflibercept (Eylea), and faricimab.

Yet further anti-angiogenesis agents include acitretin (Neotigason™), plitidepsin (Aplidine™), cilengtide (EMD 121974), combretastatin A4 (CA4P), fenretinide (4 HPR), halofuginone (Tempostatin™), Panzem™ (2-methoxyestradiol), PF-03446962 (Pfizer), rebimastat (BMS 275291), catumaxomab (Removab™), lenalidomide (Revlimid™), squalamine (EVIZON™), thalidomide (Thalomid™), Ukrain™ (NSC 631570), Vitaxin™ (MEDI 522), and zoledronic acid (Zometa™).

In other embodiments, the additional anti-cancer agent is a so-called signal transduction inhibitor (e.g., inhibiting how regulatory molecules that govern the fundamental processes of cell growth, differentiation, and survival communicated within the cell) Signal transduction inhibitors include small molecules, antibodies, and antisense molecules. Signal transduction inhibitors include for example kinase inhibitors (e.g., tyrosine kinase inhibitors or serine/threonine kinase inhibitors) and cell cycle inhibitors. More specifically signal transduction inhibitors include, for example, farnesyl protein transferase inhibitors, EGF inhibitor, ErbB-1 (EGFR), ErbB-2, pan erb, IGF1R inhibitors, MEK, c-Kit inhibitors, FLT-3 inhibitors. K-Ras inhibitors. PI3 kinase inhibitors, JAK inhibitors, STAT inhibitors. Raf kinase inhibitors, Akt inhibitors, mTOR inhibitor, P70S6 kinase inhibitors, inhibitors of the WNT pathway and so called multi-targeted kinase inhibitors. Additional examples of signal transduction inhibitors which may be used in conjunction with a compound of the invention and pharmaceutical compositions described herein include BMS 214662 (Bristol-Myers Squibb), lonafarnib (Sarasar™), pelitrexol (AG 2037), matuzumab (EMD 7200), nimotuzumab (TheraCIM h-R3™), panitumumab (Vectibix™), Vandetanib (Zactima™), pazopanib (SB 786034), ALT 110 (Alteris Therapeutics), BIBW 2992 (Boehringer ingelheim), and Cervene™ (TP 38). Other examples of signal transduction inhibitors include gefitinib (Iressa™), cetuximab (Erbitux™), erlotinib (Tarceva™), trastuzumab (Herceptin™), sunitinub (Sutent™), imatinib (Gleevec™), crizotinib (Pfizer), lorlatinib (Pfizer), dacomitinib (Pfizer), bosutinib (Pfizer), gedatolisib (Pfizer), canertinib (CI 1033), pertuzumab (Omnitarg™), lapatinib (Tycerb™), pelitinib (EKB 569), miltefosine (Miltefosin™), BMS 599626 (Bristol-Myers Squibb), Lapuleucel-T (Neuvenge™), NeuVax™ (E75 cancer vaccine), Osidem™ (IDM 1), mubritinib (TAK-165), CP-724,714 (Pfizer), panitumumab (Vectibix™), ARRY 142886 (Array Biopharm), everolimus (Certican™), zotarolimus (Endeavor™), temsirolimus (Torisel™), AP 23573 (ARIAD), and VX 680 (Vertex), XL 647 (Exelixis), sorafenib (Nexavar™), LE-AON (Georgetown University), and GI-4000 (Globelmmune). Other signal transduction inhibitors include ABT 751 (Abbott), alvocidib (flavopiridol), BMS 387032 (Bristol Myers). EM 1421 (Erimos), indisulam (E 7070), seliciclib (CYC 200), BIO 112 (One Bio), BMS 387032 (Bristol-Myers Squibb), palbociclib (Pfizer), and AG 024322 (Pfizer).

In other embodiments, the additional anti-cancer agent is a so called classical antineoplastic agent. Classical antineoplastic agents include but are not limited to hormonal modulators such as hormonal, anti-hormonal, androgen agonist, androgen antagonist and anti-estrogen therapeutic agents, histone deacetylase (HDAC) inhibitors, DNA methyltransferase inhibitors, silencing agents or gene activating agents, ribonucleases, proteosomics, Topoisomerase I inhibitors, Camptothecin derivatives, Topoisomerase 11 inhibitors, alkylating agents, antimetabolites, poly(ADP-ribose) polymerase-1 (PARP-1) inhibitor (such as, e.g., talazoparib, olapariv, rucaparib, niraparib, iniparib, veliparib), microtubulin inhibitors, antibiotics, plant derived spindle inhibitors, platinum-coordinated compounds, gene therapeutic agents, antisense oligonucleotides, vascular targeting agents (VTAs), and statins. Examples of classical antineoplastic agents used in combination therapy with a compound of the invention, optionally with one or more other agents include, but are not limited to, glucocorticoids, such as dexamethasone, prednisone, prednisolone, methylprednisolone, hydrocortisone, and progestins such as medroxyprogesterone, megestrol acetate (Megace), mifepristone (RU-486), Selective Estrogen Receptor Modulators (SERMs; such as tamoxifen, raloxifene, lasofoxifene, afimoxifene, arzoxifene, bazedoxifene, fispemifene, ormeloxifene, ospemifene, tesmihfene, toremifene, trilostane and CHF 4227 (Cheisi), Selective Estrogen-Receptor Downregulators (SERD's; such as fulvestrant, LSZ102, G1T48, RAD1901, elacestrant, GDC-9545, giredestrant, SAR439859, amcenestrant, AZD9833, camizestrant, LY3484356, Zn-c5, D-0502), exemestane (Aromasin), anastrozole (Arimidex), atamestane, fadrozole, letrozole (Femara), formestane; gonadotropin-releasing hormone (GnRH; also commonly referred to as luteinizing hormone-releasing hormone [LHRH]) agonists such as buserelin (Suprefact), goserelin (Zoladex), leuprorelin (Lupron), and triptorelin (Trelstar), abarelix (Plenaxis), cyproterone, flutamide (Eulexin), megestrol, nilutamide (Nilandron), and osaterone, dutasteride, epristeride, finasteride, Serenoa repens, PHL 00801, abarelix, goserelin, leuprorelin, triptorelin, bicalutamide; antiandrogen agents, such as enzalutamide, abiraterone acetate, bicalutamide (Casodex); and combinations thereof. Other examples of classical antineoplastic agents used in combination with a compound of the invention include but are not limited to suberolanilide hydroxamic acid (SAHA, Merck Inc./Aton Pharmaceuticals), depsipeptide (FR901228 or FK228), G2M-777, MS-275, pivaloyloxymethyl butyrate and PXD-101; Onconase (ranpirnase), PS-341 (MLN-341), Velcade (bortezomib), 9-aminocamptothecin, belotecan, BN-80915 (Roche), camptothecin, diflomotecan, edotecarin, exatecan (Daiichi), gimatecan, 10-hydroxycamptothecin, irinotecan HCl (Camptosar), lurtotecan, Orathecin (rubitecan, Supergen), SN-38, topotecan, camptothecin, 10-hydroxycamptothecin, 9-aminocamptothecin, irinotecan, SN-38, edotecarin, topotecan, aclarubicin, adriamycin, amonafide, amrubicin, annamycin, daunorubicin, doxorubicin, elsamitrucin, epirubicin, etoposide, idarubicin, galarubicin, hydroxycarbamide, nemorubicin, novantrone (mitoxantrone), pirarubicin, pixantrone, procarbazine, rebeccamycin, sobuzoxane, tafluposide, valrubicin, Zinecard (dexrazoxane), nitrogen mustard N-oxide, cyclophosphamide, AMD-473, altretamine, AP-5280, apaziquone, brostallicin, bendamustine, busulfan, carboquone, carmustine, chlorambucil, dacarbazine, estramustine, fotemustine, glufosfamide, ifosfamide, KW-2170, lomustine, mafosfamide, mechlorethamine, melphalan, mitobronitol, mitolactol, mitomycin C, mitoxatrone, nimustine, ranimustine, temozolomide, thiotepa, and platinum-coordinated alkylating compounds such as cisplatin, Paraplatin (carboplatin), eptaplatin, lobaplatin, nedaplatin. Eloxatin (oxaliplatin, Sanofi), streptozocin, satrplatin, and combinations thereof.

In still other embodiments, the additional anti-cancer agent is a so called dihydrofolate reductase inhibitors (such as methotrexate and NeuTrexin (trimetresate glucuronate)), purine antagonists (such as 6-mercaptopurine riboside, mercaptopurine, 6-thioguanine, cladribine, clofarabine (Clolar), fludarabine, nelarabine, and raltitrexed), pyrimidine antagonists (such as 5-fluorouracil (5-FU), Alimta (premetrexed disodium, LY231514, MTA), capecitabine (Xeloda™), cytosine arabinoside, Gemzar™ (gemcitabine, Eli Lilly), Tegafur (UFT Orzel or Uforal and including TS-1 combination of tegafur, gimestat and otostat), doxifluridine, carmofur, cytarabine (including ocfosfate, phosphate stearate, sustained release and liposomal forms), enocitabine, 5-azacitidine (Vidaza), decitabine, and ethylcytidine) and other antimetabolites such as eflornithine, hydroxyurea, leucovorin, nolatrexed (Thymitaq), triapine, trimetrexate, N-(5-[N-(3,4-dihydro-2-methyl-4-oxoquinazolin-6-ylmethyl)-N-methylamino]-2-thenoyl)-L-glutamic acid, AG-014699 (Pfizer Inc.). ABT-472 (Abbott Laboratories), INO-1001 (Inotek Pharmaceuticals). KU-0687 (KuDOS Pharmaceuticals) and GPI 18180 (Guilford Pharm Inc) and combinations thereof.

Other examples of classical antineoplastic cytotoxic agents include, but are not limited to, Abraxane (Abraxis BioScience, Inc.), Batabulin (Amgen), EPO 906 (Novartis), Vinflunine (Bristol-Myers Squibb Company), actinomycin D, bleomycin, mitomycin C, neocarzinostatin (Zinostatin), vinblastine, vincristine, vindesine, vinorelbine (Navelbine), docetaxel (Taxotere), Ortataxel, paclitaxel (including Taxoprexin a DHA/paclitaxel conjugate), cisplatin, carboplatin, Nedaplatin, oxaliplatin (Eloxatin), Satraplatin, Camptosar, capecitabine (Xeloda), oxaliplatin (Eloxatin), Taxotere alitretinoin, Canfosfamide (Telcyta™), DMXAA (Antisoma), ibandronic acid, L-asparaginase, pegaspargase (Oncaspar™), Efaproxiral (Efaproxyn™—radiation therapy), bexarotene (Targretin™), Tesmilifene (DPPE—enhances efficacy of cytotoxics), Theratope™ (Biomira), Tretinoin (Vesanoid™), tirapazamine (Trizaone™), motexafin gadolinium (Xcytrin™) Cotara™ (mAb), and NBI-3001 (Protox Therapeutics), polyglutamate-paclitaxel (Xyotax™) and combinations thereof. Further examples of classical antineoplastic agents include, but are not limited to, as Advexin (ING 201), TNFerade (GeneVec, a compound which express TNFalpha in response to radiotherapy), RB94 (Baylor College of Medicine), Genasense (Oblimersen, Genta), Combretastatin A4P (CA4P), Oxi-4503, AVE-8062, ZD-6126, TZT-1027, Atorvastatin (Lipitor, Pfizer Inc.), Provastatin (Pravachol, Bristol-Myers Squibb), Lovastatin (Mevacor, Merck Inc.), Simvastatin (Zocor, Merck Inc.), Fluvastatin (Lescol, Novartis), Cerivastatin (Baycol, Bayer), Rosuvastatin (Crestor, AstraZeneca), Lovostatin, Niacin (Advicor, Kos Pharmaceuticals), Caduet, Lipitor, torcetrapib, and combinations thereof.

In other embodiments, the additional anti-cancer agent is an epigenetic modulator, for example an inhibitor or EZH2, SMARCA4, PBRM1, ARID1A, ARID2, ARID1B, DNMT3A, TET2, MLL1/2/3, NSD1/2, SETD2, BRD4, DOT1L, HKMTsanti, PRMT1-9, LSD1, UTX, IDH1/2 or BCL6.

In further embodiments, the additional anti-cancer agent is an immunomodulatory agent, such as, but not limited to, an inhibitor of CTLA-4 (e.g., ipilimumab). PD-1 or PD-L1 (e.g., pembrolizumab, nivolumab, avelumab, atezolizumab, durvalumab, cemiplimab, or dosterlimab), LAG-3 (e.g., relatlimab), TIM-3, TIGIT, 4-1BB, OX40, GITR, CD40, or a CAR-T-cell therapy.

In some embodiments, the additional anticancer agent is an EGFR inhibitor such as afatinib, osimertinib, lapatinib, erlotinib, dacomitinib, poziotinib, neratinib or gefitinib or an EGFR antibody such as cetuximab, panitumumab, or necitumumab.

Alternatively, a compound of the disclosure, a pharmaceutically acceptable salt thereof or a pharmaceutical composition disclosed herein can be administered in combination with other anti-cancer agents that are not EGFR inhibitors e.g., in combination with MEK, including mutant MEK inhibitors (trametinib, cobimtetinib, binimetinib, selumetinib, refametinib); c-MET, including mutant c-Met inhibitors (savolitinib, cabozantinib, foretinib) and MET antibodies (emibetuzumab); mitotic kinase inhibitors (CDK4/6 inhibitors such as palbociclib, ribociclib, abemacicilb, lerociclib, trilaciclib, dalpiciclib, BPI-16350); anti-angiogenic agents e.g., bevacizumab, nintedanib; apoptosis inducers such as Bcl-2 inhibitors e.g., venetoclax, obatoclax, navitoclax and Mcl-1 inhibitors e.g., AZD-5991, AMG-176, S-64315; and mTOR inhibitors e.g., rapamycin, temsirolimus, everolimus, ridoforolimus.

A compound of the disclosure, a pharmaceutically acceptable salt thereof or a pharmaceutical composition disclosed herein can also be administered in combination with an effective amount of a second agent selected from the group consisting of palbociclib (e.g., Ibrance®), ribociclib, abemaciclib, tamoxifen, letrozole, olaparib (e.g., Lynparza®), niraparib, carboplatin, cisplatin, paclitaxel, gemcitabine, megestrol acetate, medroxyprogesterone acetate, capecitabine (e.g., Xeloda®), regorafenib (e.g., Stivarga®), afatinib (e.g., Gilotrif®), osimertinib (e.g., Tagrisso®), gefitinib (e.g., Iressa®), erlotinib (e.g., Tarceva®), ramucirumab (e.g., Cyramza®), an EGFR inhibitor, pralsetinib, ABT-263 (navitoclax), MK-1775 (adavosertib), BAY-1895344, berzosertib, ceralasertib, SRA-737, LY2603618 (rabusertib), and trastuzumab (e.g., Herceptin®), or combinations thereof. The EGFR inhibitor may be selected from afatinib, osimertinib, lapatinib, erlotinib, dacomitinib, poziotinib, neratinib, gefitinib JBJ-04-125-02, alflutinib (AST 2818), aumolertinib (formerly almonertinib) (HS10296), BBT-176, BI-4020, BPI-361175, BPI-D0316, CH7233163, gilitertinib, icotinib, JND-3229, lazertinib, nazartinib (EGF 816), avitinib, PCC-0208027, rezivertinib (BPI-7711). TQB3804, zorifertinib (AZ-3759), or DZD9008; an EGFR antibody such as cetuximab, panitumumab, necitumumab, HLX07, JMT101; or a bispecific EGFR and MET antibody (e.g., amivantamab ((JNJ-61186372, JNJ-372)).

### Biomarkers and Pharmacodynamics Markers

The disclosure further provides predictive markers (e.g., biomarkers and pharmacodynamic markers. e.g., gene copy number, gene sequence, expression levels, or phosphorylation levels) to identify those human subjects having, suspected of having, or at risk of developing a disease or disorder associated with CDK2 for whom administering a CDK2 inhibitor (“a CDK2 inhibitor” as used herein refers to a compound of the disclosure, or a pharmaceutically acceptable salt thereof) is likely to be effective.

### CCNE1

In one embodiment, the biomarker is CCNE1. In particular an amplification of the cyclin E1 (CCNE1) gene and/or an expression level of CCNE1 in a biological sample would indicate that the patient or subject could benefit from administration of a compound of Formula (I) or Formula (I-1) or a pharmaceutically acceptable salt thereof.

CCNE1 is a cell cycle factor essential for the control of the cell cycle at the G1/S transition (Ohtsubo et al., 1995, Mol. Cell. Biol. 15:2612-2624). CCNE1 acts as a regulatory subunit of CDK2, interacting with CDK2 to form a serine/threonine kinase holoenzyme complex. The CCNE1 subunit of this holoenzyme complex provides the substrate specificity of the complex (Honda et al., 2005, EMBO 24: 452-463) CCNE1 is encoded by the cyclin E1 (“CCNE1”) gene (GenBank Accession No. NM_001238). The amino acid sequence of human CCNE1 is found at GenBank Accession No. NP_001229/UniProtKB Accession No. P24864).

In one aspect, the present disclosure provides a method of treating a subject having, or at risk of developing, a disease or disorder associated with CDK2, comprising administering to the subject a therapeutically effective amount of a compound disclosed herein, or a pharmaceutically acceptable salt thereof, or a pharmaceutical composition disclosed herein, wherein the subject has an amplification of the CCNE1 gene and/or have an expression level of CCNE1 higher than a control expression level of CCNE1 In some embodiments, the disease or disorder associated with CDK2 is cancer.

Also provided herein is a method of treating a patient having an amplified expression level of CCNE1 and suffering from, or at risk of developing, a solid tumor cancer, comprising administering to the patient a therapeutically effective amount of a compound disclosed herein, or a pharmaceutically acceptable salt thereof, or a pharmaceutical composition disclosed herein.

An amplification of the CCNE1 gene and/or an expression level of CCNE1 that is higher than a control expression level of CCNE1 is indicative/predictive that a human subject having or at risk of developing a disease or disorder associated with CDK2 will respond to a CDK2 inhibitor. In some embodiments, the expression level of CCNE1 may be the level of CCNE1 mRNA. In other embodiments, the expression level of CCNE1 may be the level of CCNE1 protein.

### Other Biomarkers

In some embodiments, the contemplated biomarker may be p16 (also known as cyclin-dependent kinase inhibitor 2A, cyclin-dependent kinase 4 inhibitor A, multiple tumor suppressor 1, and p16-INK4a), which acts as a negative regulator of the proliferation of normal cells by interacting with CDK4 and CDK6. In other embodiments, the contemplated biomarker may be phosphorylation of Rb at the serine corresponding to amino acid position 780. Rb is a regulator of the cell cycle and acts as a tumor suppressor. Rb is activated upon phosphorylation by cyclin D-CDK4/6 at Ser780 and Ser795 and by cyclin E/CDK2 at Ser807 and Ser811.

The contemplated biomarker may also be selected from the group consisting of RB1, RBL1, RBL2, CDKN2A, CDKN1A, CDKN1B, FBXW7, CCNE1, CCNE2, CCNA1, CCNA2, CCND1, CCND2, CCND3, CDK2, CDK3, CDK4, CDK6, CDKN2A, CDNK1A, CDKN1B E2F1, E2F2, E2F3, MYC, MYCL, MYCN, EZH2, ER, HER2, HER3, HPV+, and EGFR.

### Biological Samples

Suitable biological samples for the methods described herein include any sample that contains blood or tumor cells obtained or derived from the human subject in need of treatment. For example, a biological sample can contain tumor cells from biopsy from a patient suffering from a solid tumor. A tumor biopsy can be obtained by a variety of means known in the art. Alternatively, a blood sample can be obtained from a patient suffering from a hematological cancer.

A biological sample can be obtained from a human subject having, suspected of having, or at risk of developing, a disease or disorder associated with CDK2. In some embodiments, the disease or disorder associated with CDK2 is a cancer (such as those described supra).

Methods for obtaining and/or storing samples that preserve the activity or integrity of molecules (e.g., nucleic acids or proteins) in the sample are well known to those skilled in the art. For example, a biological sample can be further contacted with one or more additional agents such as buffers and/or inhibitors, including one or more of nuclease, protease, and phosphatase inhibitors, which preserve or minimize changes in the molecules in the sample.

### Methods of Administration and Dosage Forms

The precise amount of compound administered to provide an “effective amount” to the subject will depend on the mode of administration, the type, and severity of the cancer, and on the characteristics of the subject, such as general health, age, sex, body weight, and tolerance to drugs. The skilled artisan will be able to determine appropriate dosages depending on these and other factors. When administered in combination with other therapeutic agents, e.g., when administered in combination with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of Formula (I) or Formula (I-1) being used by following, for example, dosages reported in the literature and recommended in the Physician's Desk Reference (57th Ed., 2003).

“Treating” or “treatment” refers to obtaining a desired pharmacological and/or physiological effect. The effect can be therapeutic, which includes achieving, partially or substantially, one or more of the following results: partially or substantially reducing the extent of the disease, condition or cancer; ameliorating or improving a clinical symptom or indicator associated with the disease, condition or cancer; delaying, inhibiting or decreasing the likelihood of the progression of the disease, condition or cancer; or decreasing the likelihood of recurrence of the disease, condition or cancer.

The term “effective amount” means an amount when administered to the subject which results in beneficial or desired results, including clinical results, e.g., inhibits, suppresses or reduces the symptoms of the condition being treated in the subject as compared to a control. For example, a therapeutically effective amount can be given in unit dosage form (e.g., 0.1 mg to about 50 g per day, alternatively from 1 mg to about 5 grams per day; and in another alternatively from 10 mg to 1 gram per day).

The terms “administer”, “administering”, “administration”, and the like, as used herein, refer to methods that may be used to enable delivery of compositions to the desired site of biological action. These methods include, but are not limited to, intraarticular (in the joints), intravenous, intramuscular, intratumoral, intradermal, intraperitoneal, subcutaneous, orally, topically, intrathecally, inhalationally, transdermally, rectally, and the like. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa.

In addition, a compound of the disclosure, a pharmaceutically acceptable salt thereof or a pharmaceutical composition of the disclosure can be co-administered with other therapeutic agents. As used herein, the terms “co-administration”, “administered in combination with”, and their grammatical equivalents, are meant to encompass administration of two or more therapeutic agents to a single subject, and are intended to include treatment regimens in which the agents are administered by the same or different route of administration or at the same or different times. In some embodiments the one or more compounds of the disclosure, a pharmaceutically acceptable salt thereof or a pharmaceutical composition of the disclosure will be co-administered with other agents. These terms encompass administration of two or more agents to the subject so that both agents and/or their metabolites are present in the subject at the same time. They include simultaneous administration in separate compositions, administration at different times in separate compositions, and/or administration in a composition in which both agents are present. Thus, in some embodiments, the compounds described herein and the other agent(s) are administered in a single composition. In some embodiments, the compounds described herein and the other agent(s) are admixed in the composition.

The particular mode of administration and the dosage regimen will be selected by the attending clinician, taking into account the particulars of the case (e.g., the subject, the disease, the disease state involved, the particular treatment). Treatment can involve daily or multi-daily or less than daily (such as weekly or monthly etc.) doses over a period of a few days to months, or even years. However, a person of ordinary skill in the art would immediately recognize appropriate and/or equivalent doses looking at dosages of approved compositions for treating a disease using the disclosed CDK2 inhibitors for guidance.

The compounds of the disclosure or a pharmaceutically acceptable salt thereof can be administered to a patient in a variety of forms depending on the selected route of administration, as will be understood by those skilled in the art. The compounds of the present teachings may be administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration can be by continuous infusion over a selected period of time.

The pharmaceutical composition of the disclosure is formulated to be compatible with its intended route of administration. In an embodiment, the composition is formulated in accordance with routine procedures as a pharmaceutical composition adapted for intravenous, subcutaneous, intramuscular, oral, intranasal, or topical administration to human beings. In preferred embodiments, the pharmaceutical composition is formulated for intravenous administration.

Typically, for oral therapeutic administration, a compound of the disclosure or a pharmaceutically acceptable salt thereof may be incorporated with excipient and used in the form of ingestible tablets, buccal tablets, troches, capsules, elixirs, suspensions, syrups, wafers, and the like.

Typically for parenteral administration, solutions of a compound of the disclosure can generally or a pharmaceutically acceptable salt thereof be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms.

Typically, for injectable use, sterile aqueous solutions or dispersion of, and sterile powders of, a compound of the disclosure for the extemporaneous preparation of sterile injectable solutions or dispersions are appropriate.

The following examples are intended to be illustrative and are not intended to be limiting in any way to the scope of the disclosure.

### EXEMPLIFICATION

### Preparation of Exemplary Compounds

### Definitions

AcOH means acetic acid;
t-AmOH means tert-amyl alcohol;
Aq. means aqueous;
Bn means benzyl;
Boc means tert-butoxy carbonyl;
Boc₂O means di-tert-butyl dicarbonate;
(BPin)₂ means 4,4,4′,4′,5,5,5′,5′-Octamethyl-2,2′-bi-1,3,2-dioxaborolane;
br means broad;
Brettphos means 2-(Dicyclohexylphosphino)3,6-dimethoxy-2′,4′,6′-triisopropyl-1,1′-biphenyl;
BrettPhos Pd G3 means [(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate;
n-BuOH means butan-1-ol;
t-BuOH means tertiary butanol;
t-BuOK means potassium tert-butoxide;
t-BuXPhos Pd G3 means (2-Di-tert-butylphosphino-2′,4′,6′-triisopropyl-1,1′-biphenyl)-2-(2′-amino-1,1′-biphenyl) palladium(11) methanesulfonate;
° C. means degrees Celsius;
CDCl₃ means deutero-chloroform;
Cs₂CO₃ means cesium carbonate;
CuCN means copper cyande;
δ means chemical shift;
d means doublet;
dd means doublet of doublets;
dq means doublet of quartets;
dt means doublet of triplets;
DAST means Diethylaminosulfur trifluoride;
DBU means 1,8-diazabicyclo[5.4.0]undec-7-ene;
DCM means dichloromethane;
DEA means diethylamine;
DEAD means diethyl azodicarboxylate;
DIAD means diisopropyl azodicarboxylate;
DIBAL-H means diisobutylaluminium hydride;
DIPEA means N-ethyldiisopropylamine or N,N-diisopropylethylamine;
DMA means N,N-Dimethylacetamide;
DMF means N,N-dimethylformamide;
DMSO means Dimethylsulfoxide;
DMSO-d₆ means hexadeuterodimethyl sulfoxide;
EA means ethyl acetate;
Et means ethyl;
Et₂O means diethyl ether;
EtOAc means ethyl acetate;
EtOH means ethanol;
Eq. means equivalent;
g means gram;
HATU means 1-[bis(dimethylamino)methylene]-1H-1,2,3-triazolo[4,5-h]pyridinium 3-oxid hexafluorophosphate;
HBF₄ means tetrafluoroboric acid;
HCl means hydrochloric acid;
HCOH means formaldehyde;
HCO₂H means formic acid;
Hept means heptet;
¹H NMR means proton nuclear magnetic resonance;
H₂O means water;
H₂O₂ means hydrogen peroxide;
HPLC means high pressure liquid chromatography;
h means hour;
IPA means 2-propanol;
K₂CO₃ means potassium carbonate;
KI means potassium iodide;
KOH means potassium hydroxide;
K₃PO₄ means potassium phosphate tribasic;
L means litre;
LCMS means liquid chromatography mass spectrometry;
LDA means lithium diisopropylamide;
LiAlH₄ means lithium aluminium hydride;
LiOH means lithium hydroxide;
in means multiplet;
M means molar;
Me means methyl;
MeCN means acetonitrile;
MeI means iodomethane;
MeLi means methyl lithium;
MeMgBr means methyl magnesium bromide;
MeNH₂ means methylamine;
MeOH means methanol;
MeOH-d₄ means deutero-methanol;
mg means milligram;
MgSO₄ means magnesium sulfate;
MHz means mega Hertz;
mins means minutes;
mL means millilitres;
mmol means millimole;
MPLC means medium pressure liquid chromatography;
MS m/z means mass spectrum peak;
MTBE means methyl tert-butyl ether;
N₂ means nitrogen;
NaBH₄ means sodium borohydride;
Na₂CO₃ means sodium carbonate;
NaH means sodium hydride;
NaHCO₃ means sodium bicarbonate;
NaOH means sodium hydroxide;
Na₂SO₄ means sodium sulfate;
NCS means N-chlorosuccinimide;
NH₃ means ammonia;
NH₄Cl means ammonium chloride;
NH₄HCO₃ means ammonium carbonate;
NH₂OH means hydroxylamine;
NH₄OH is ammonium hydroxide;
NMP means N-methyl pyrrolidine;
PE means petroleum ether;
Pd(amphos)Cl₂ means Bis(di-tert-butyl(4-dimethylaminophenyl)phosphine)dichloropalladium(II);
Pd(t-Bu₃P)₂ means Bis(tri-tert-butylphosphine)palladium(t);
Pd(OAc) means palladium acetate;
Pd₂(dba)₃ means tris(dibenzylideneacetone)dipalladium (0);
Pd(dppf)Cl₂ means [1,1′-bis(diphenylphosphino)ferrocene]dichloropalladium(II);
Pd(PPh₃)₄ means tetrakis(triphenylphosphine)palladium(0);
Pd(PPh₃)Cl₂ means Palladium(II)bis(triphenylphosphine)dichloride;
Pd/C means palladium on charcoal;
Pd(OH)₂ means palladium hydroxide;
PPh₃ means triphenylphosphine;
q means quartet;
rt means room temperature;
RT means retention time;
RuPhos Pd G3 means (2-dicyclohexylphosphino-2′,6′-diisopropoxy-1,1′-biphenyl)[2-(2′-amino-1,1′-biphenyl)]palladium(II) methanesulfonate;
s means singlet;
sat, means saturated;
SFC means supercritical fluid chromatography;
soln. means solution;
t means triplet;
TBAF means Tetrabutylammonium fluoride;
TBDMSCl means tert-Butyl(chloro)dimethylsilane;
TEA means triethylamine;
TFA means trifluoroacetic acid;
TfOH means trifluoromethanesulfonic acid;
THF means tetrahydrofuran;
TLC means thin layer chromatography;
TsOH means p-toluenesulfonic acid;
μL means micro litres;
μmol means micromole;
Xantphos means 4,5-Bis(diphenylphosphino)-9,9-dimethylxanthene;
Xantphos Pd G2 means Chloro[(4,5-bis(diphenylphosphino)-9,9-dimethylxanthene)-2-(2′-amino-1,1′-biphenyl)]palladium(II);
Xantphos Pd G3 means [(4,5-Bis(diphenylphosphino)-9,9-dimethylxanthene)-2-(2′-amino-1,1′-biphenyl)]palladium(I) methanesulfonate;
XPhos Pd G2 means Chloro(2-dicyclohexylphosphino-2′,4′,6′-triisopropyl-1,1′-biphenyl)[2-(2′-amino-1,1′-biphenyl)]palladium(II).

Methods for preparing compounds of the invention can be carried out in suitable solvents which can be readily selected by one of skill in the art of organic synthesis. Suitable solvents can be substantially non-reactive with the starting materials (reactants), intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in Wuts and Greene, Protective Groups in Organic Synthesis, 5th ed., John Wiley & Sons: New Jersey, (2014), which is incorporated herein by reference in its entirety.

Reactions can be monitored according to any suitable method known in the art. For example, product formation can be monitored by spectroscopic means, such as nuclear magnetic resonance (NMR) spectroscopy (e.g., 1H or 13C), infrared (IR) spectroscopy, spectrophotometry (e.g., UV-visible), mass spectrometry (MS), or by chromatographic methods such as high performance liquid chromatography (HPLC) or thin layer chromatography (TLC). Representative analytical instruments and methods for compound characterization can be used as follows unless specifically described in the Examples:

LC-MS: The liquid chromatography-mass spectrometry (LC-MS) data can be obtained with an Agilent Technologies 1200 Series LCMSD utilizing APT-ESI ionization fitted with a reverse-phase column (Sunfire CIS, 3.5 um particle size, 4.6×50 mm dimensions) at 50 degrees Celsius. The mobile phase may consist of a mixture of solvent 0.01% TFA in water and 0.01% TFA in acetonitrile. A constant gradient from 5% increase to 95% organic within 1.3 min, 95% organic for 1.7 min can be utilized with a flow rate constant at 2 mL/min. Alternatively, the liquid chromatography-mass spectrometry (LC-MS) data can be obtained with an Agilent Technologies 1200 Series LCMSD utilizing API-ESI ionization utilizing ESI ionization fitted with a reverse-phase column (XBridge C18, 3.5 um particle size, 4.6×50 mm dimensions) at 45 degrees Celsius. The mobile phase may consist of a mixture of solvent 10 mM NH4HCO3 in water and acetonitrile. A constant gradient from 5% increase to 95% organic within 1.4 min, 95% organic for 1.6 min can be utilized with a flow rate constant at 1.8 mL/min.

Prep LC-MS: Preparative HPLC can be performed on a Gilson 281 Preparative system fitted with a Welch Xtimate 10 u C18 100A, AXIA packed, 250×21.2 mm reverse-phase column at 20 degrees Celsius. The mobile phase can consist of a mixture of solvent 0.1% formic acid in water and 0.1% formic acid in acetonitrile. A constant gradient from 70% aqueous/30% organic to 30% aqueous/70% organic mobile phase over the course of 15 minutes can be utilized, with a flow rate constant at 30 mL/min. Alternatively, fitted with Column: Welch Xtimate 10 u C18 21.2*250 mm, 10 um; The mobile phase can consist of a mixture of solvent Water (10 mmol/L NH4HCO3+0.05% NH3·H2O) and acetonitrile. A constant gradient from 70% aqueous/30% organic to 30% aqueous/70% organic mobile phase over the course of 15 minutes can be utilized, with a flow rate constant at 30 mL/min.

Silica gel chromatography: Silica gel chromatography can be performed on a Biotage® Isolera One unit, or a Biotage® Isolera Prime unit.

Proton NMR: 1H NMR spectra can be obtained with a Bruker AVANCE III 400 MHz, 400 MHz NMR instrument (acquisition time=3.16 seconds with a 1 second delay; 8 to 32 scans) or a Bruker AVANCE III 400 MHz, 400 MHz NMR instrument (acquisition time=3.98 seconds with a 1 second delay; 8 to 32 scans) or a Bruker AVANCE III 500 MHz 500 MHz NMR instrument (acquisition time=3.17 seconds with a 1 second delay; 8 to 32 scans). Unless otherwise indicated, all protons were reported in DMSO-d6 solvent as parts-per million (ppm) with respect to residual DMSO (2.50 ppm).

SFC: Waters Preparative system (SFC80, SFC150, SFC200, SFC350).

Chiral-HPLC: Gilson 281 (vendor: GILSON)

One of ordinary skill in the art will recognize that modifications of the gradient, column length, and flow rate are possible and that some conditions may be more suitable for compound characterization than others, depending on the chemical species being analyzed.

### Example 1. (1R,3S)-3-(3-((5-cyanopyrazin-2-yl)amino)-1H-pyrazol-5-yl)cyclopentyl(1-methylcyclopropyl)carbamate

### Step 1. Synthesis of benzyl (1-(tert-butyl)-3-(3-hydroxycyclopentyl)-1H-pyrazol-5-yl)carbamate (Intermediate 1)

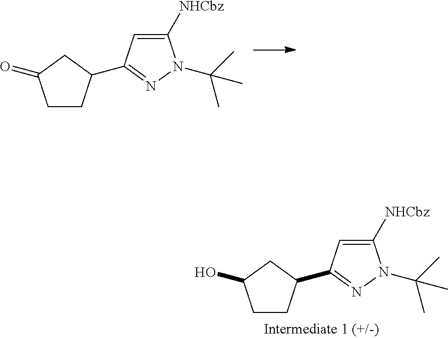

Two batches were carried out in parallel. To a solution of benzyl (1-(tert-butyl)-3-(3-oxocyclopentyl)-1H-pyrazol-5-yl)carbamate (140 g, 394 mmol) in THF (150 mL) was added LiBHEt₃ (1 M, 591 mL) at −65° C. and the mixture was stirred at −65° C. for 1 hr. The two batches were combined and the resulting mixture was poured into water (2.50 L) at 20˜25° C. and extracted with ethyl acetate (2.00 L*3). The combined organic layer was washed with HCl (1 M, 1.20 L), brine (1.00 L*2), and concentrated. The residue was triturated with petroleum ether:ethyl acetate=1:1 (1.20 L, 5.00 V) at 25° C. for 2 hrs. The suspension was filtered and the filter cake was dried under vacuum to give intermediate 1 (210 g, 70.6% yield) as a white solid. MS (ES+) C₂₀H₂₇N₃O₃ requires: 357 found: 358 [M+H]⁺.

### Step 2. Synthesis of benzyl (1-(tert-butyl)-3-((1S,3R)-3-hydroxycyclopentyl)-1H-pyrazol-5-yl)carbamate (Intermediate 2)

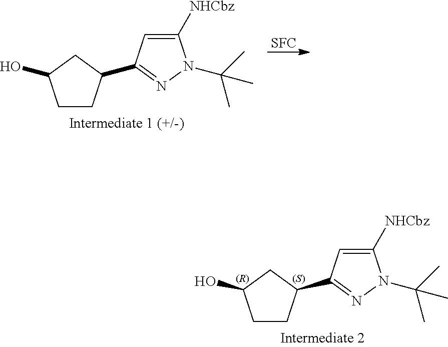

Intermediate 1 (210 g, 588 mmol) was separated by SFC (column: DAICEL CHIRALPAK AD (250 mm*50 mm, 10 urn); mobile phase: [Neu−MeOH]; B %: 20%-20%, 2 min). The desired fraction was concentrated to give intermediate 2 (72.0 g, 33.9% yield) as a white solid, which was confirmed by HPLC, SFC, and ¹H NMR. HPLC: product Rt=1.065 mins; SFC: product: Rt=1.891 mins, ee %=98.4% under 220 nm; ¹H NMR: EC4266-3-P1A (400 MHz DMSO-d₆) δ 9.06 (s, 1H), 7.38-7.32 (m, 5H), 5.92 (s, 1H), 5.12 (s, 2H), 4.56 (d, J=4.4 Hz, 1H), 4.16-4.12 (m, 1H), 2.91-2.85 (m, 1H), 2.22-2.15 (m, 1H), 1.85-1.83 (m, 1H), 1.73-1.69 (m, 2H), 1.59-1.50 (m, 2H), 1.47 (s, 9H).

### Step 3. Synthesis of benzyl (1-(tert-butyl)-3-((1S,3R)-3-(((4-nitrophenoxy)carbonyl)oxy)cyclopentyl)-1H-pyrazol-5-yl)carbamate (Intermediate 3)

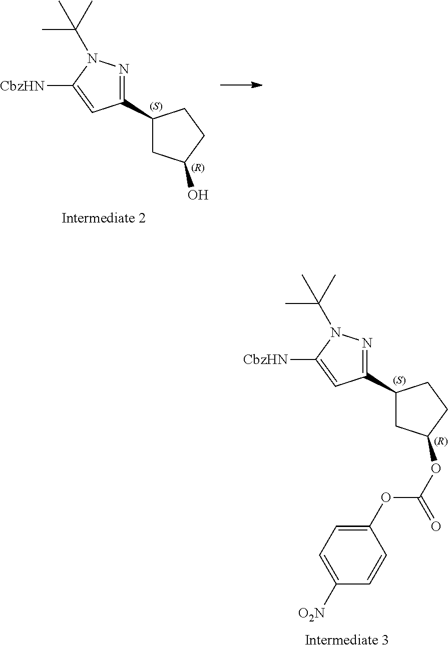

Two batches were carried out in parallel. A solution of intermediate 2 (20.0 g, 55.9 mmol) and 4-nitrophenyl chloroformate (14.7 g, 72.7 mmol) in DCM (200 mL) was stirred at 25° C. under N₂ and was then cooled to 0° C. To the mixture was added pyridine (13.3 g, 167.9 mmol) and DMAP (684 mg, 5.60 mmol) at 0° C. The reaction mixture was stirred at 25° C. for 2 hrs. The two reaction batches were combined and the mixture Was concentrated and purified by silica gel column chromatography (petroleum ether:ethyl acetate=from 30:1 to 2:1, petroleum ether:ethyl acetate=3:1). The desired fraction was concentrated to give the intermediate 3 (58.0 g, crude) as a yellow gum. MS (ES+) (C₂₇H₃₀N₄O₇ requires: 522, found: 523 [M+H]⁺.

### Step 4: Synthesis of benzyl (1-(tert-butyl)-3((1S,3R)-3-(((1-methylcyclopropyl)carbamoyl)oxy)cyclopentyl)-1H-pyrazol-5-yl)carbamate (Intermediate 4)

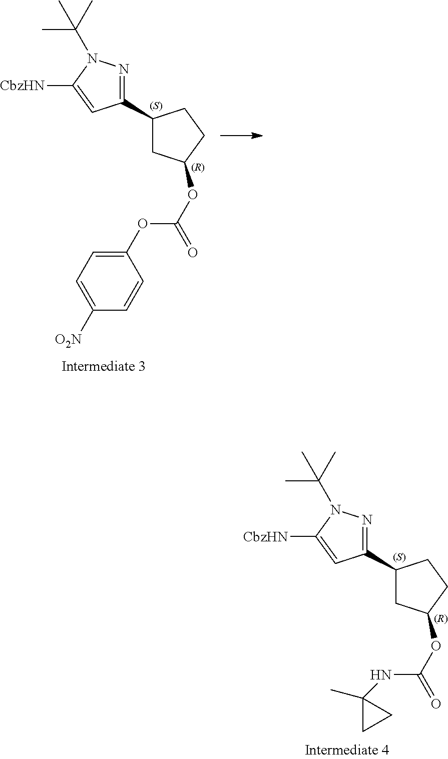

To a solution of intermediate 3 (525 g, 90.4 mmol) in THF (520 mL) was added 1-methylcyclopropan-1-amine hydrochloride (19.5 g, 181 mmol) and DIEA (58.4 g, 452 mmol) at 25° C., the mixture was heated to 60° C. and stirred at 60° C. for 12 hrs. The mixture was concentrated and the resulting residue was diluted with ethyl acetate (1.50 L). The mixture was washed with H₂O (500 mL), sat. Na₂CO₃ (500 mL), brine (500 mL), and dried over Na₂SO₄. The residue was filtered and concentrated under reduced pressure and was purified by column chromatography (SiO₂ petroleum ether:ethyl acetate=from 50:1 to 2:1, petroleum ether:ethyl acetate=2:1). The desired fractions were concentrated to give intermediate 4 (33.8 g, 95.8% purity) as a white solid. MS (ES+) C₂₅H₃₄N₄O₄ requires: 454, found: 455 [M+H]⁺.

### Step 5. Synthesis of benzyl (3-((1S,3R)-3-(((1-methylcyclopropyl)carbamoyl)oxy)cyclopentyl)-1H-pyrazol-5-yl)carbamate (Intermediate 5)

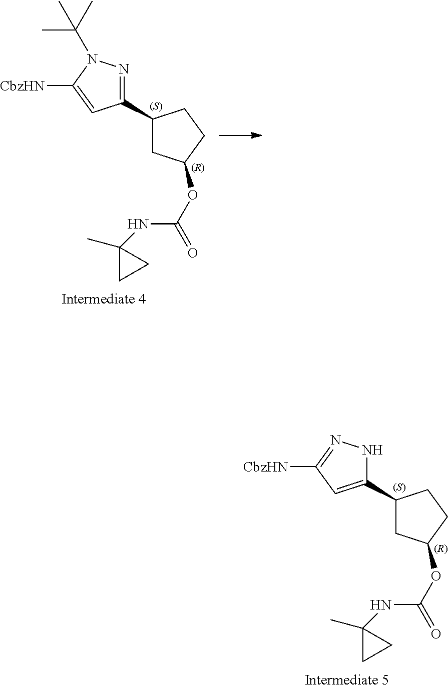

A solution of intermediate 4 (29.0 g, 63.8 mmol) in formic acid (145 mL) was stirred at 75° C. for 14 hrs. The mixture was concentrated and the resulting residue was diluted with a mixture of ethyl acetate:tetrahydrofuran (10:1, 800 mL) and washed with sat·NaHCO₃ (300 mL), and brine (300 mL). The organic phase was separated and dried with Na₂SO₄, filtered, and concentrated. The residue was then triturated with a mixture of ethyl acetate:methanol (10:1, 200 mL) at 25° C. for 1 hr. The mixture was filtered and the filter cake was dried under vacuum to give the intermediate 5 (14.8 g, 55.9% yield) as a white solid. MS (ES+) C₂₁H₂₆N₄O₄ requires: 398, found: 399 [M+H]⁺.

### Step 6. Synthesis of (1R,3S)-3-(5-amino-1H-pyrazol-3-yl)cyclopentyl(1-methylcyclopropyl)carbamate (Intermediate 6)

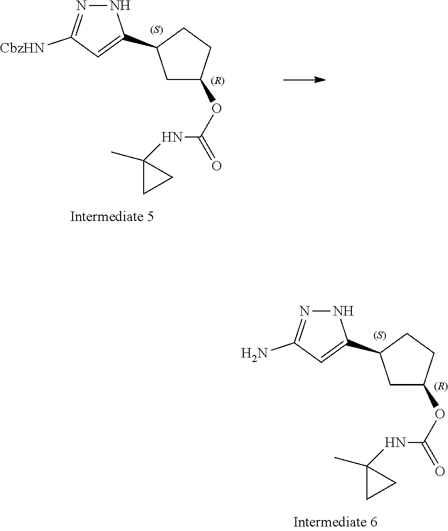

To a solution of intermediate 5 (11.8 g, 29.6 mmol) in MeOH (220 mL) was added Pd/C (12.0 g, 29.6 mmol) and the mixture was degassed and purged with H₂ (3×). The mixture was heated to 50° C. and stirred for 12 hrs under H₂ (50 psi). The mixture was filtered and concentrated to give the crude intermediate 6 (7.90 g, crude) as a gray solid that was used in the next step without purification. MS (ES+) C₁₃H₂₀N₄O₂ requires: 264, found: 265 [M+H]⁺.

### Step 7. Synthesis of (1R,3S)-3-(3-((5-cyanopyrazin-2-yl)amino)-1H-pyrazol-5-yl)cyclopentyl(1-methylcyclopropyl)carbamate (Example 1)

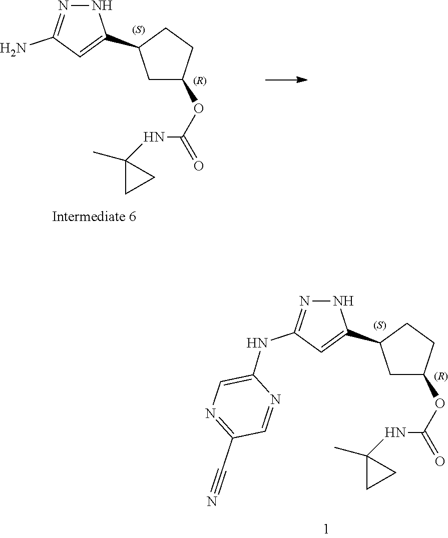

A mixture of Intermediate 6 (820 mg, 3.11 mmol) 5-chloropyrazine-2-carbonitrile (518 mg, 3.73 mmol), BrettPhos Pd G4 (230 mg, 0.16 mmol), and potassium acetate (914 mg, 9.33 mmol) in dioxane (30 mL) was stirred at 90° C. for 4 h under N₂. The reaction mixture was cooled to RT and concentrated to give a residue which was purified by silica gel chromatography (PE:EA=1:9) and then by Prep-HPLC (Mobile phase: A=water (0.1% NH₄HCO₃), B:=acetonitrile; Gradient: B:=15%-95% in 18 min; Column: Xtimate 10 um 150A 21.2×250 mm) to afford the title compound (190.5 mg, 16% yield) as a yellow solid. MS (ES+) C₁₈H₂₁N₇O₂ requires: 367, found: 368 [M+H]⁺. ¹H-NMR (500 MHz, DMSO-d₆) δ ppm 12.26 (s, 1H), 10.63 (s, 1H), 8.62 (s, 1H) 8.45 (s, 1H), 7.34 (s, 1H), 6.33 (s, 1H), 4.98 (s, 1H), 3.15-3.07 (m, 1H), 2.48-2.45 (m, 1H), 2.07-1.54 (m, 1H), 1.23 (m, 1H), 0.60-0.46 (m, 4H).

### Example 2. Synthesis of (1R,3S)-3-(3-((4-cyanopyridin-2-yl)amino)-1H-pyrazol-5-yl)cyclopentyl(1-methylcyclopropyl)carbamate

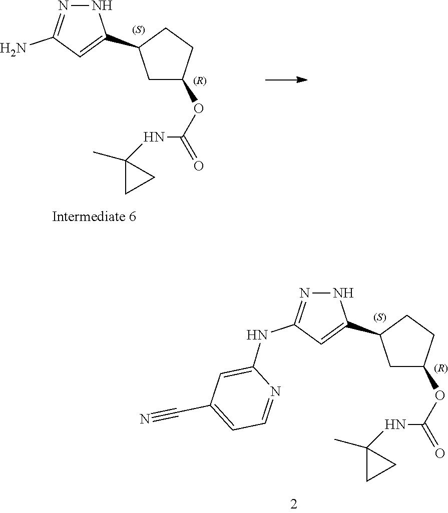

The title compound was prepared using an analogous method to that described for Example 1 using 2-chloroisonicotinonitrile. The desired compound was purified by silica gel chromatography (DCM:MeOH=20:1) and then by Prep-HPLC (Mobile phase: A=water (0.1% NH₄HCO₃), B=acetonitrile; Gradient: B=15%-95% in 18 min; Column: Xtimate 10 um 150A 21.2×250 min) to afford the title compound (13.6 mg, 12% yield) as a yellow solid. MS (ES+) C₁₉H₂₂N₆O₂ requires: 366, found: 367 [M+H]⁺. ¹H-NMR (400 MHz, DMSO-d₆) δ ppm 11.98 (s, 1H), 9.70 (s, 1H), 8.31 (d, J=5.2 Hz, 1H), 7.65 (s, 1H), 7.36 (s, 1H), 7.03 (d, J=5.2 Hz, 1H), 6.07 (s, 1H), 4.98 (s, 1H), 3.05-3.02 (m, 1H), 2.51-2.44 (m, 1H), 2.02-1.69 (m, 5H), 1.23 (s, 1H), 0.61-0.57 (m, 2H), 0.48-0.46 (m, 2H).

### Example 3. Synthesis of (1R,3S)-3-(3-((2-cyanopyrimidin-4-yl)amino)-1H-pyrazol-5-yl)cyclopentyl(1-methylcyclopropyl)carbamate

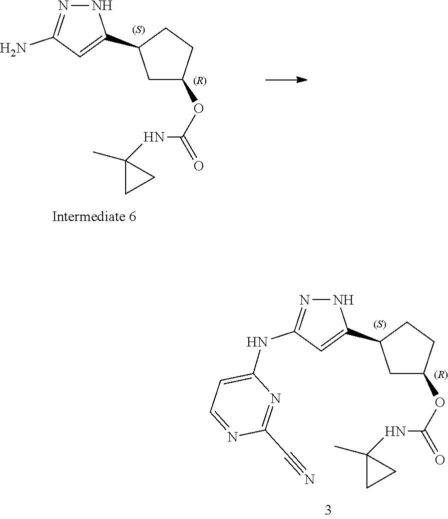

The title compound was prepared using an analogous method to that described for Example 1 using 4-chloropyrimidine-2-carbonitrile. The desired compound was purified by silica gel chromatography (DCM:MeOH=20:1) and then by Prep-HPLC (Mobile phase: A=water (0.1% NH₄HCO₃), B=acetonitrile: Gradient: B=15%-95% in 18 min; Column: Xtimate 10 um 150A 21.2×250 mm) to afford the title compound (5.2 mg, 4.6% yield). MS (ES+) C₁₈H₂₁N₇O₂ requires: 367, found: 368 [M+H]⁺. ¹H-NMR (500 MHz, DMSO-d₆) δ ppm 12.27 (s, 1H), 10.46 (s, 1H), 8.36 (s, 1H), 7.33 (s, 1H), 4.99 (s, 1H), 3.09-3.06 (m, 1H), 2.02-1.57 (m, 5H), 1.23 (s, 1H), 0.61-0.57 (m, 2H), 0.48-0.45 (m, 2H).

### Example 4: Synthesis of (1R,3S)-3-(3-((5-cyano-3-methylpyrazin-2-yl)amino)-1H-pyrazol-5-yl)cyclopentyl(1-methylcyclopropyl)carbamate

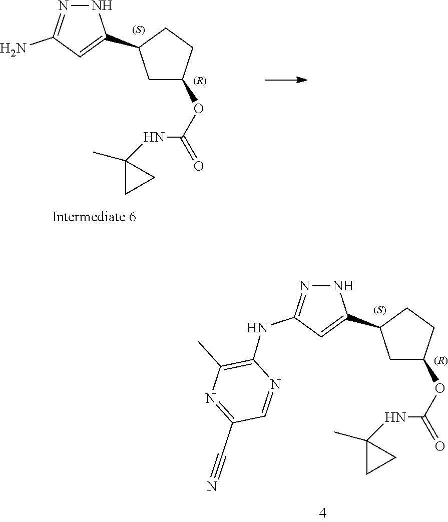

The title compound was prepared using an analogous method to that described for Example 1 using 5-chloro-6-methylpyrazine-2-carbonitrile. The desired compound was purified by flash chromatography eluting with EA to give a crude product. The crude product was further purified via Prep-HPLC (Mobile phase: A=water (0.1% Formic Acid), B Acetonitrile; Gradient: B=5-95% B in 8.0 min; Column: Welch 10 um 150A 21.2*250 mm) to afford the title compound (291 mg, 39% yield) as a white solid. MS (ES+) C₁₉H₂₃N₇O₂ requires: 381, found: 382 [M+H]⁺. ¹H-NMR (400 MHz, DMSO-d₆) δ ppm 12.27 (s, 1H), 9.56 (s, 1H), 8.49 (s, 1H), 7.36 (s, 1H), 6.41 (s, 1H), 5.00 (s, 1H), 3.08-3.05 (m, 1H), 2.04-2.03 (m, 1H), 1.90-1.80 (m, 1H), 1.72-1.70 (m, 2H), 1.62-1.53 (m, 1H), 1.23 (s, 3H), 0.60-0.57 (m, 2H), 0.48-0.45 (m, 2H).

### Example 5: Synthesis of (1R,3S)-3-(3-((5-cyanopyridazin-3-yl)amino)-1H-pyrazol-5-yl)cyclopentyl(1-methyl cyclopropyl)carbamate

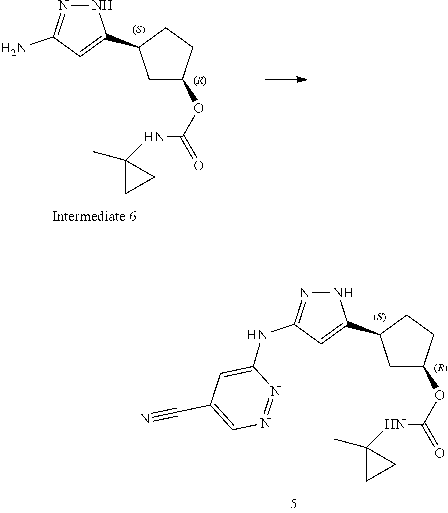

The title compound was prepared using an analogous method to that described for Example 1 using 6-chloropyridazine-4-carbonitrile. The desired compound was purified by flash chromatography on silica gel eluting with EA/PE (10/1) and then by Prep-HPLC (Mobile phase: A=water(0.1% NH₄HCO₃), B=acetonitrile; Gradient: B:=15%-95% in 18 min; Column: Xtimate 10 um 150A 21.2×250 mm) to obtain the title product (11.4 mg, 10% yield) as the yellow solid. MS (ES+) C₁₈H₂₁N₇O₂ requires: 367, found: 368 [M+H]⁺. ¹H-NMR (500 MHz, DMSO-d₆) δ ppm 12.11 (s, 1H), 10.15 (s, 1H), 8.91 (s, 1H), 7.94 (s, 1H), 7.34 (s, 1H), 6.17 (s, 1H), 4.99 (s, 1H), 3.15-3.00 (m, 1H), 2.47-2.43 (m, 1H), 2.08-1.97 (m, 1H), 1.97-1.85 (m, 1H), 1.85-1.65 (m, 2H), 1.65-1.53 (m, 1H), 1.23 (s, 3H), 0.58-0.47 (m, 4H).

### Example 6-20

The title compounds were prepared from (Intermediate 6) and the appropriate halo-heterocycle (Het) using an analogous procedure as described for Example 1.

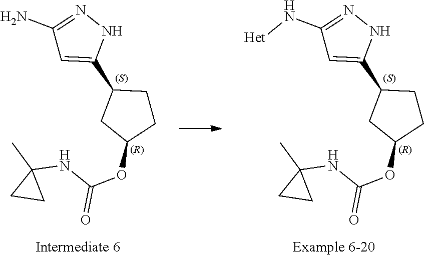

### Step 1. Synthesis of 2-(5-chloropyrazin-2-yl)propan-2-ol (Intermediate 7)

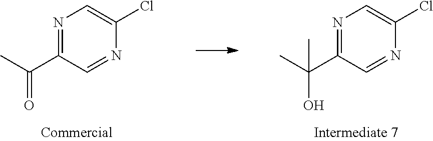

To a solution of 1-(5-chloropyrazin-2-yl)ethan-1-one (200 mg, 1.27 mmol) in THF (4 mL) was added methyl magnesium bromide (3.0 M in THF, 0.5 mL) at 0° C., and then stirred for 1 h at rt. The reaction was quenched With 1M NH₄C and extracted with EtOAc. The combined organic layer was washed with brine, dried (Na₂SO₄) and concentrated in vacuo. The residue was purified by flash chromatography on silica gel (2:1 PE/EtOAc) to afford the title compound as a yellow oil (70 mg, 31% yield). ¹H-NMR (400 MHz, DMSO-d₆) δ ppm 8.60 (s 1H) 8.50 (s 1H) 3.37 (br. s., 1H), 1.61 (s, 6H).

| Number | Name, Structure, Het, Data |
|---|---|
| 6 | (1R,3S)-3-(3-((3-methylpyrazin-2-yl)amino)-1H-pyrazol-5-yl)cyclopentyl |
| | (1-methylcyclopropyl)carbamate |
| | 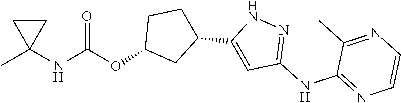 [SMILES: Cc1nccnc1Nc1cc([C@H]2CC[C@@H](OC(=O)NC3(C)CC3)C2)[nH]n1] |
| | Het: 2-chloro-3-methylpyrazine |
| | Prep-HPLC (Xtimate, 21.2 x 250 mm, 10 mm; 15-95% MeCN/H₂O (0.1% |
| | NH₄HCO₃)); Yellow solid (24.3 mg, 22.5% yield). |
| | MS (ES+) C₁₈H₂₄N₆O₂ requires: 356, found: 357 [M + H]⁺. ¹H-NMR (500 |
| | MHz, DMSO-d₆) δ ppm 11.98 (br. s, 1H), 8.61 (s, 1H), 7.94 (s, 1H), 7.78 (d, |
| | J = 2.0 Hz, 1H), 7.33 (s, 1H), 6.31 (s, 1H), 4.98 (s, 1H), 3.06-3.04 (m, 1H), |
| | 2.45 (s, 3H), 2.02-1.60 (m, 5H), 1.22 (s, 3H), 0.59-0.57 (m, 2H), 0.47-0.45 |
| | (m, 2H). |
| 7 | (1R,3S)-3-(3-((6-methylpyrazin-2-yl)amino)-1H-pyrazol-5-yl)cyclopentyl |
| | (1-methylcyclopropyl)carbamate |
| | 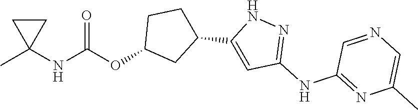 |
| | Het: 2-chloro-6-methylpyrazine |
| | Prep-HPLC (Welch, 21.2 x 250 mm, 10 mm; 25-50% MeCN/H₂O (0.1% |
| | HCO₂H)); White solid (37.4 mg, 18% yield). |
| | MS (ES+) C₁₈H₂₄N₆O₂ requires: 356, found: 357 [M + H]⁺. 1H-NMR (500 |
| | MHz, DMSO-d₆) δ ppm 11.937 (br. s., 1H), 9.43 (s, 1H), 8.42 (s, 1H), 7.78 |
| | (s, 1H), 7.34 (s, 1H), 6.12 (br. s., 1H), 4.98 (s, 1H), 3.19-3.16 (m, 1H), 2.48 |
| | (s, 1H), 1.89-1.71 (m, 3H), 1.69-1.58 (m, 3H), 1.23 (s, 3H), 0.58-0.57 (m, |
| | 2H), 0.48-0.46 ( m, 2H). |
| 8 | (1R,3S)-3-(3-((5-(difluoromethoxy)pyrazin-2-yl)amino)-1H-pyrazol-5- |
| | yl)cyclopentyl (1-methylcyclopropyl)carbamate |
| | 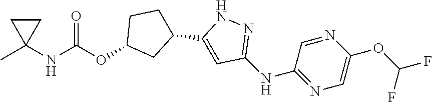 [SMILES: CC1(NC(=O)O[C@@H]2CC[C@H](c3cc(Nc4cnc(OC(F)F)cn4)n[nH]3)C2)CC1] |
| | Het: 2-chloro-5-(difluoromethoxy)pyrazine |
| | Prep-HPLC (Xtimate, 21.2 x 250 mm, 10 mm; 15-95% MeCN/H₂O (0.1% |
| | NH₄HCO₃)); White solid (17.8 mg, 23% yield). |
| | MS (ES+) C₁₈H₂₂F₂N₆O₃ requires: 408, found: 409 [M + H]⁺. ¹H-NMR (500 |
| | MHz, DMSO-d₆) δ ppm 11.92 (br. s., 1H), 9.57 (s, 1H), 8.33 (s, 1H), 8.06 |
| | (s, 1H), 7.44 (t, J = 73.0 Hz, 1H), 7.34 (s, 1H), 6.04 (s, 1H), 4.97 (s, 1H), |
| | 3.05-3.02 (m, 1H), 2.01-1.99 (m, 1H), 1.90-1.84 (m, 1H), 1.70-1.57 (m, |
| | 3H), 1.23 (s, 3H), 0.60-0.46 (m, 4H). |
| 9 | (1R,3S)-3-(3-((5-cyano-6-methylpyrazin-2-yl)amino)-1H-pyrazol-5- |
| | yl)cyclopentyl (1-methylcyclopropyl)carbamate |
| | 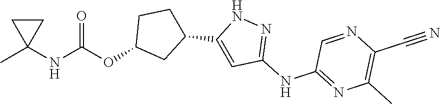 [SMILES: Cc1nc(Nc2cc([C@H]3CC[C@@H](OC(=O)NC4(C)CC4)C3)[nH]n2)cnc1C#N] |
| | Het: 5-chloro-3-methylpyrazine-2-carbonitrile |
| | Prep-HPLC (Xtimate, 21.2 x 250 mm, 10 mm; 15-95% MeCN/H₂O (0.1% |
| | NH₄HCO₃)); Yellow solid (23.9 mg, 20% yield). |
| | MS (ES+) C₁₉H₂₃N₇O₂ requires: 381, found: 382 [M + H]⁺. ¹H-NMR (400 |
| | MHz, DMSO-d₆) δ ppm 12.21 (s, 1H), 10.52 (s, 1H), 8.38 (s, 1H), 7.33 (s, |
| | 1H), 6.31 (s, 1H), 4.99 (s, 1H), 3.08-3.06 (m, 1H), 2.51 (s, 3H), 2.03-1.59 |
| | (m, 5H), 1.23 (s, 3H), 0.58-0.56 (m, 2H), 0.48-0.46 (m, 2H). |
| 10 | (1R,3S)-3-(3-((2-(trifluoromethyl)pyrimidin-4-yl)amino)-1H-pyrazol-5- |
| | yl)cyclopentyl (1-methylcyclopropyl)carbamate |
| | 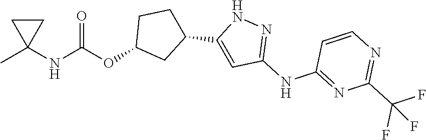 [SMILES: CC1(NC(=O)O[C@@H]2CC[C@H](c3cc(Nc4ccnc(C(F)(F)F)n4)n[nH]3)C2)CC1] |
| | Het: 4-chloro-2-(trifluoromethyl)pyrimidine |
| | Prep-HPLC (Xtimate, 21.2 x 250 mm, 10 mm; 15-95% MeCN/H₂O (0.1% |
| | NH₄HCO₃)); White solid (23.4 mg, 10% yield). |
| | MS (ES+) C₁₈H₂₁F₃N₆O₂ requires: 410, found: 411 [M + H]⁺. ¹H-NMR (400 |
| | MHz, DMSO-d₆) δ ppm 12.23 (s, 1H), 10.44 (s, 1H), 8.42 (s, 1H), 7.31 (s, |
| | 1H), 4.98 (s, 1H), 3.08-3.05 (m, 1H), 2.02-2.01 (m, 1H), 1.91-1.87 (m, 1H), |
| | 1.70-1.68 (m, 2H), 1.58-1.56 (m, 1H), 1.23 (s, 3H), 0.58-0.46 (m, 4H). |
| 11 | (1R,3S)-3-(3-((6-cyanopyrazin-2-yl)amino)-1H-pyrazol-5-yl)cyclopentyl (1- |
| | methylcyclopropyl)carbamate |
| | 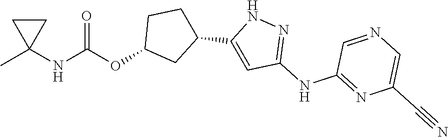 [SMILES: CC1(NC(=O)O[C@@H]2CC[C@H](c3cc(Nc4cncc(C#N)n4)n[nH]3)C2)CC1] |
| | Het: 6-chloropyrazine-2-carbonitrile |
| | Prep-HPLC (Xtimate, 21.2 x 250 mm, 10 mm; 15-95% MeCN/H₂O (0.1% |
| | NH₄HCO₃)); Yellow solid (39 mg, 14% yield). |
| | MS (ES+) C₁₈H₂₁N₇O₂ requires: 367, found: 368 [M + H]⁺. ¹H-NMR (400 |
| | MHz, DMSO-d₆) δ ppm 12.15 (br. s., 1H), 10.28 (s, 1H), 8.70 (s, 1H), 8.37 |
| | (s, 1H), 7.32 (s, 1H), 6.22 (s, 1H), 4.99 (s, 1H), 3.09-3.06 (m, 1H), 2.03-2.02 |
| | (m, 1H), 1.89-1.85 (m, 1H), 1.71-1.65 (m, 2H), 1.58-1.56 (m, 1H), 1.23 (s, |
| | 3H), 0.60-0.45 (m, 4H). |
| 12 | (1R,3S)-3-(3-((5-cyanopyridin-2-yl)amino)-1H-pyrazol-5-yl)cyclopentyl (1- |
| | methylcyclopropyl)carbamate |
| | 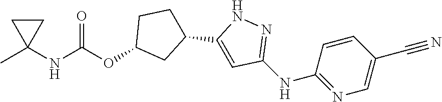 [SMILES: CC1(NC(=O)O[C@@H]2CC[C@H](c3cc(Nc4ccc(C#N)cn4)n[nH]3)C2)CC1] |
| | Het: 6-chloronicotinonitrile |
| | Prep-HPLC (Xtimate, 21.2 x 250 mm, 10 mm; 15-95% MeCN/H₂O (0.1% |
| | NH₄HCO₃)); Yellow solid (8.7 mg, 7% yield). |
| | MS (ES+) C₁₉H₂₂N₆O₂ requires: 366, found: 367 [M + H]⁺. ¹H-NMR (500 |
| | MHz, DMSO-d₆) δ ppm 12.06 (s, 1H), 9.97(s, 1H), 8.51 (s, 1H), 7.87 (dd, |
| | J = 9.0 Hz, 2.0 Hz, 1H), 7.33 (s, 1H), 7.28-7.26 (m, 1H), 6.17 (s, 1H), 4.98 (s, |
| | 1H), 3.05-3.03 (m, 1H), 2.49-2.46 (m, 1H), 2.02-1.57 (m, 6H), 1.23 (s, 3H), |
| | 0.60-0.58 (m, 2H), 0.48-0.46 (m, 2H). |
| 13 | (1R,3S)-3-(3-(pyridin-2-ylamino)-1H-pyrazol-5-yl)cyclopentyl (1- |
| | methylcyclopropyl)carbamate |
| | 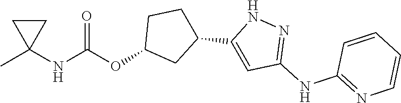 [SMILES: CC1(NC(=O)O[C@@H]2CC[C@H](c3cc(Nc4ccccn4)n[nH]3)C2)CC1] |
| | Het: 2-chloropyridine |
| | Prep-HPLC (Xtimate, 21.2 x 250 mm, 10 mm; 15-95% MeCN/H₂O (0.1% |
| | NH₄HCO₃)); Yellow solid (8 mg, 7% yield). |
| | MS (ES+) C₁₈H₂₃N₅O₂ requires: 341, found: 342 [M + H]⁺. ¹H-NMR (400 |
| | MHz, DMSO-d₆) δ ppm 11.78 (s, 1H), 9.05 (s, 1H), 8.08 (d, J = 4.0 Hz, |
| | 1H), 7.51 (t, J = 6.8 Hz, 1H), 7.33 (s, 1H), 7.20 (s, 1H), 6.66 (t, J = 6.0 Hz, |
| | 1H), 6.06 (s, 1H), 4.99 (s, 1H), 3.03-3.01 (m, 1H), 2.49-2.43 (m, 1H), 2.01- |
| | 1.58 (m, 5H), 1.23 (s, 3H), 0.61-0.58 (m, 2H), 0.48-0.47 (m, 2H). |
| 14 | (1R,3S)-3-(3-((4-cyano-3-methylpyridin-2-yl)amino)-1H-pyrazol-5- |
| | yl)cyclopentyl (1-methylcyclopropyl)carbamate |
| | 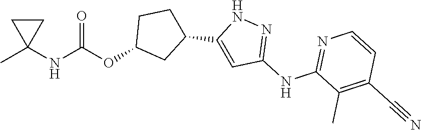 [SMILES: Cc1c(C#N)ccnc1Nc1cc([C@H]2CC[C@@H](OC(=O)NC3(C)CC3)C2)[nH]n1] |
| | Het: 2-chloro-3-methylpyridine-4-carbonitrile |
| | Prep-HPLC (Xtimate, 21.2 x 250 mm, 10 mm; 15-95% MeCN/H₂O (0.1% |
| | NH₄HCO₃)); White solid (16.3 mg, 8% yield). |
| | MS (ES+) C₂₀H₂₄N₆O₂ requires: 380, found: 381 [M + H]⁺. ¹H-NMR (500 |
| | MHz, DMSO-d₆) δ ppm 12.00 (br. s., 1H), 8.65 (s, 1H), 8.13 (s, 1H), 7.33 |
| | (s, 1H), 7.01 (d, J = 5.0 Hz, 1H), 6.28 (s, 1H), 4.98 (s, 1H), 3.05-3.02 (m, |
| | 1H), 2.45-2.43 (m, 1H), 2.39(s, 3H), 2.00-1.99 (m, 1H), 1.88-1.85 (m, 1H), |
| | 1.70-1.60 (m, 3H), 1.22 (s, 3H), 0.59-0.46 (m, 4H). |
| 15 | (1R,3S)-3-(3-((4-cyano-6-methylpyridin-2-yl)amino)-1H-pyrazol-5- |
| | yl)cyclopentyl (1-methylcyclopropyl)carbamate |
| | 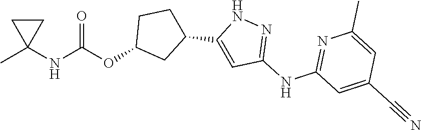 [SMILES: Cc1cc(C#N)cc(Nc2cc([C@H]3CC[C@@H](OC(=O)NC4(C)CC4)C3)[nH]n2)n1] |
| | Het: 2-chloro-6-methylisonicotinonitrile |
| | Prep-HPLC (Xtimate, 21.2 x 250 mm, 10 mm; 15-95% MeCN/H₂O (0.1% |
| | NH₄HCO₃)); Yellow solid (23.9 mg, 20% yield). |
| | MS (ES+) C₂₀H₂₄N₆O₂ requires: 380, found: 381 [M + H]⁺. ¹H-NMR (400 |
| | MHz, DMSO-d₆) δ ppm 11.93 (s, 1H), 9.60 (s, 1H), 7.57 (s, 1H), 7.33 (s, |
| | 1H), 6.92 (s, 1H), 5.99 (s, 1H), 4.98 (s, 1H), 3.05-3.03 (m, 1H), 2.38 (s, 3H), |
| | 2.02-1.54 (m, 6H), 1.23 (s, 3H), 0.59-0.57 (m, 2H), 0.47-0.45 (m, 2H). |
| 16 | (1R,3S)-3-(3-((6-cyanopyrimidin-4-yl)amino)-1H-pyrazol-5-yl)cyclopentyl |
| | (1-methylcyclopropyl)carbamate |
| | 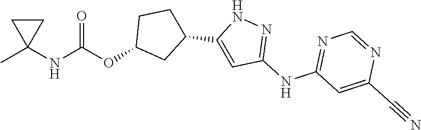 [SMILES: CC1(NC(=O)O[C@@H]2CC[C@H](c3cc(Nc4cc(C#N)ncn4)n[nH]3)C2)CC1] |
| | Het: 6-chloropyrimidine-4-carbonitrile |
| | Prep-HPLC (Xtimate, 21.2 x 250 mm, 10 mm; 15-95% MeCN/H₂O (0.1% |
| | NH₄HCO₃)); Yellow solid (16.1 mg, 14% yield). |
| | MS (ES+) C₁₈H₂₁N₇O₂ requires: 367, found: 368 [M + H]⁺. ¹H-NMR (400 |
| | MHz, DMSO-d₆) δ ppm 12.26 (s, 1H), 10.54 (s, 1H), 8.67 (s, 1H), 7.34 (s, |
| | 1H), 4.98 (s, 1H), 3.07-3.05 (m, 1H), 2.46-2.44 (m, 1H), 2.03-1.86 (m, 5H), |
| | 1.23 (s, 3H), 0.60-0.58 (m, 2H), 0.49-0.45 (m, 2H). |
| 17 | (1R,3S)-3-(3-((5-(2-hydroxypropan-2-yl)pyrazin-2-yl)amino)-1H-pyrazol-5- |
| | yl)cyclopentyl (1-methylcyclopropyl)carbamate |
| | 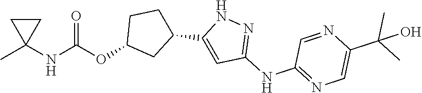 [SMILES: CC1(NC(=O)O[C@@H]2CC[C@H](c3cc(Nc4cnc(C(C)(C)O)cn4)n[nH]3)C2)CC1] |
| | Het: 2-(5-chloropyrazin-2-yl)propan-2-ol (Intermediate 7) |
| | Prep-HPLC (BOSTON pHlex, 21.2 x 250 mm, 10 m mm; 20-40% |
| | MeCN/H₂O (0.1% HCO₂H)); White solid (20.6 mg, 12% yield). |
| | MS (ES+) C₂₀H₂₈N₆O₃ requires: 400, found: 401 [M + H]⁺, ¹H-NMR (400 |
| | MHz, DMSO-d₆) δ ppm 11.91 (br. s, 1H), 9.43 (s, 1H), 8.46 (s, 1H), |
| | 8.31 (s, 1H), 7.35 (s, 1H), 6.11 (s, 1H), 5.13 (s, 1H), 4.99 (s, 1H), |
| | 3.20-3.01 (m, 1H), 2.01-1.99 (m, 1H), 1.99-1.88 (m, 1H), 1.71-1.69 |
| | (m, 2H), 1.69-1.67 (m, 1H), 1.42 (s, 6H), 1.23 (s, 3H), 0.59-0.57 (m, |
| | 2H), 0.48-0.46 (m, 2H). |
| 18 | (1R,3S)-3-(3-((6-cyanopyridazin-3-yl)amino)-1H-pyrazol-5-yl)cyclopentyl |
| | (1-methylcyclopropyl)carbamate |
| | 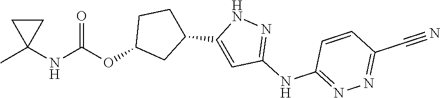 [SMILES: CC1(NC(=O)O[C@@H]2CC[C@H](c3cc(Nc4ccc(C#N)nn4)n[nH]3)C2)CC1] |
| | Het: 6-chloropyridazine-3-carbonitrile |
| | Prep-HPLC (Xtimate, 21.2 x 250 mm, 10 mm; 15-95% MeCN/H₂O (0.1% |
| | NH₄HCO₃)); Yellow solid (48.5 mg, 44% yield). |
| | MS (ES+) C₁₈H₂₁N₇O₂ requires: 367, found: 368 [M + H]⁺. ¹H-NMR (400 |
| | MHz, DMSO-d₆) δ ppm 12.23 (br. s., 1H), 10.48 (s, 1H), 7.94-7.92 (m, 1H), |
| | 7.71 (s, 1H), 7.35 (s, 1H), 6.18 (s, 2H), 4.99 (s, 1H), 3.09 (s, 1H), 2.03-1.56 |
| | (m, 6H), 1.23 (s, 3H), 0.59-0.47 (m, 4H). |
| 19 | (1R,3S)-3-(3-((2-cyano-5-methylpyrimidin-4-yl)amino)-1H-pyrazol-5- |
| | yl)cyclopentyl (1-methylcyclopropyl)carbamate |
| | 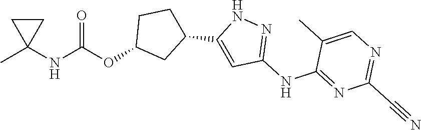 [SMILES: Cc1cnc(C#N)nc1Nc1cc([C@H]2CC[C@@H](OC(=O)NC3(C)CC3)C2)[nH]n1] |
| | Het: 4-chloro-5-methylpyrimidine-2-carbonitrile |
| | Prep-HPLC (Xtimate, 21.2 x 250 mm, 10 mm; 15-95% MeCN/H₂O (0.1% |
| | NH₄HCO₃)); White solid (20.1 mg, 11% yield). |
| | MS (ES+) C₁₉H₂₃N₇O₂ requires: 381, found: 382 [M + H]⁺. ¹H-NMR (400 |
| | MHz, DMSO-d₆) δ ppm 12.29 (s, 1H), 9.47 (s, 1H), 8.22 (s, 1H), 7.35 (s, |
| | 1H), 6.37 (s, 1H), 5.00 (s, 1H), 3.11-3.09 (m, 1H), 2.22 (s, 3H), 2.08-1.61 |
| | (m, 6H), 1.24 (s, 3H), 0.59-0.47 (m, 4H). |
| 20 | (1R,3S)-3-(3-((5-cyano-3-methylpyridin-2-yl)amino)-1H-pyrazol-5- |
| | yl)cyclopentyl (1-methylcyclopropyl)carbamate |
| | 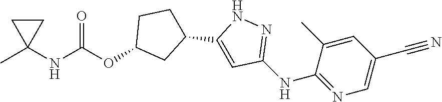 [SMILES: Cc1cc(C#N)cnc1Nc1cc([C@H]2CC[C@@H](OC(=O)NC3(C)CC3)C2)[nH]n1] |
| | Het: 6-chloro-5-methylpyridine-3-carbonitrile |
| | Prep-HPLC (BOSTON pHlex, 21.2 x 250 mm, 10 m mm; 25-50% |
| | MeCN/H₂O (0.1% HCO₂H)); White solid (28.6 mg, 19% yield). |
| | MS (ES+) C₂₀H₂₄N₂O₆ requires: 380, found: 381 [M + H]⁺. ¹H-NMR (400 |
| | MHz, DMSO-d₆) δ ppm 12.12 (br. s, 1H), 8.88 (s, 1H), 8.39 (s, 1H), 7.73 (s, |
| | 1H), 7.36 (s, 1H), 6.34 (s, 1H), 4.99 (s, 1H), 3.10-3.06 (m, 1H), 2.22 (s, 3H), |
| | 2.22-2.05 (m, 1H), 2.02-1.89 (m, 1H), 1.80-1.69 (m, 2H), 1.68-1.60 (m, 1H), |
| | 1.23 (s, 3H), 0.60-0.58 (m, 2H), 0.48-0.46 (m, 2H). |

### Example 21 and 22. (1R,3S)-3-(3-((5-((S)-1-hydroxyethyl)pyrazin-2-yl)amino)-1H-pyrazol-5-yl)cyclopentyl(1-methylcyclopropyl)carbamate and (1R,3S)-3-(3-((5-((R)-1-hydroxyethyl)pyrazin-2-yl)amino)-1H-pyrazol-5-yl)cyclopentyl(1-methylcyclopropyl)carbamate

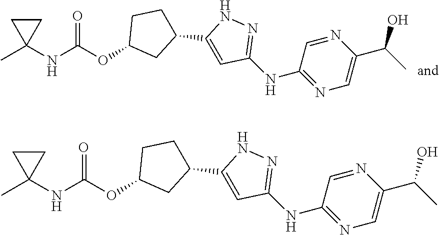

### Step 1. Synthesis of (1R,3S-3-(3-((5-acetylpyrazine-2-yl)amino)-1H-pyrazol-5-yl)cyclopentyl(1-methylcyclopropyl)carbamate (Intermediate 8)

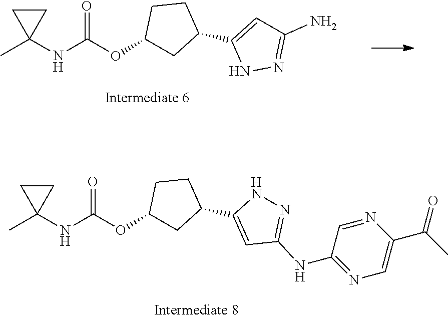 [SMILES: CC1(NC(=O)O[C@@H]2CC[C@H](c3cc(N)n[nH]3)C2)CC1 ; CC(=O)c1cnc(Nc2cc([C@H]3CC[C@@H](OC(=O)NC4(C)CC4)C3)[nH]n2)cn1]

A mixture of (Intermediate 6, 150 mg, 568 μmol), 1-(5-chloropyrazin-2-yl)ethan-1-one (128 rug, 852 μmol), BrettPhos Pd G4 (15 mg; 9.76 μmol) and KOAc (281 mg, 2.87 mmol) in dioxane (5 mL) was stirred at 90° C. for 8 h under nitrogen. The mixture was evaporated to dryness in vacuo and the residue purified by flash chromatography on silica gel (1:2 EtOAc/PE) to afford the title compound (Intermediate 8) as a yellow oil (150 mg, 68%). MS (ES+) C₁₉H₂₄N₆O₃ requires: 384, found: 385 [M+H]⁺.

### Step 2. Synthesis of (1R,3S)-3-(3-((5-((S)-1-hydroxyethyl)pyrazin-2-yl)amino)-1H-pyrazol-5-yl)cyclopentyl(1-methylcyclopropyl)carbamate and (1R,3S)-3-(3-((5-((R)-1-hydroxyethyl)pyrazin-2-yl)amino)-1H-pyrazol-5-yl)cyclopentyl(1-methylcyclopropyl)carbamate (Example 21 and 22)

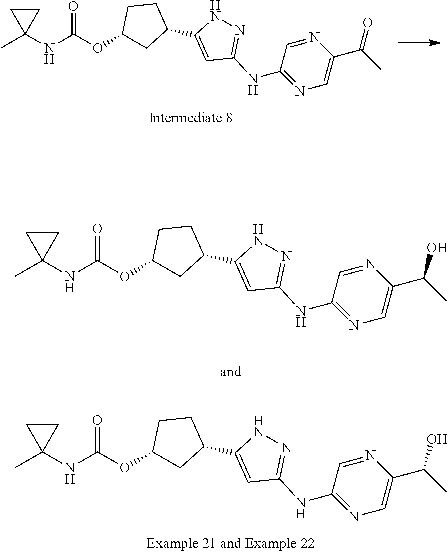

To a solution of Intermediate 8 (80 mg, 208 mol) in MeOH (5 ml) was added NaBH₄ (10.2 mg, 270 μmol) at 0° C. and then stirred at rt for 1 h. The reaction mixture was diluted with EtOAc and washed with water. The organic layer was evaporated to dryness in vacuo and the residue purified by flash chromatography on silica, gel followed by prep-HPLC (Xtimate, 21.2×250 mm, 10 mm; 15-95% MeCN/H₂O (0.1% NH₄HCO₃)) to afford the racemic mixture (˜55 mg). The racemate was purified by chiral prep-SFC (Diacel AS, 20×250 mm, 10 mm; 25% MeOH (0.2% NH₃) in CO₂) to afford:

Peak 1, Example 21 (13.6 mg, 17% yield). (1R,3S)-3-(3-((5-((S)-1-hydroxyethyl)pyrazin-2-yl)amino)-1H-pyrazol-5-yl)cyclopentyl(1-methylcyclopropyl)carbamate or (1R,3S)-3-(3-((5-((R)-1-hydroxyethyl)pyrazin-2-yl)amino)-1H-pyrazol-5-yl)cyclopentyl(1-methylcyclopropyl)carbamate. MS (ES+) C₁₉H₂₆N₆O₃ requires: 386, found: 387 [M+H]⁺; ¹H-NMR (400 MHz, DMSO-d₆) δ ppm 11.94 (br. s., 1H), 9.48 (s, 1H), 8.48 (s, 1H), 8.16 (s, 1H), 7.37 (s, 1H), 6.13 (s, 1H), 5.24-5.23 (m, 1H), 4.99-4.98 (m, 1H), 4.69-4.66 (m, 1H), 3.04-3.02 (m 1H), 2.02-1.99 (m, 2H), 1.81-1.79 (m, 2H) 1.75-1.69 (m, 2H), 1.35 (d, J=6.8 Hz, 3H), 1.23 (s, 3H), 0.61-0.57 (m, 2H), 0.47-0.45 (m, 2H).

Peak 2, Example 22 (13.7 mg, 17% yield). (1R,3S)-3-(3-((5-((S)-1-hydroxyethyl)pyrazin-2-yl)amino)-1H-pyrazol-5-yl)cyclopentyl(1-methylcyclopropyl)carbamate or (1R,3S)-3-(3-((5-((R)-1-hydroxyethyl)pyrazin-2-yl)amino)-1H-pyrazol-5-yl)cyclopentyl(1-methylcyclopropyl)carbamate. MS (ES+) C₁₉H₂₆N₆O₃ requires: 386, found: 387 [M+H]⁺; ¹H-NMR (400 MHz, DMSO-d₆) δ ppm 11.99 (br. s., 1H), 9.50 (s, 1H), 8.47 (s, 1H), 8.16 (s, 1H), 7.37 (s, 1H), 6.14 (s, 1H), 5.2.4-5.23 (m, 1H), 4.99-4.98 (m, 1H), 4.72-4.70 (m, 1H) 3.06-3.05 (m, 1H), 2.01-1.70 (m, 6H), 1.35 (d, J=6.8 Hz, 3H), 1.23 (s, 3H), 0.61-0.57 (m, 2H), 0.47-0.45 (m, 2H).

### Example 23. (1R,3S)-3-(3-((3-(methylamino)-1,2,4-triazin-5-yl)amino)-1H-pyrazol-5-yl)cyclopentyl(1-methylcyclopropyl)carbamate

### Step 1. Synthesis of (1R,3S)-3-(5-amino-1-(tert-butyl)-1H-pyrazol-3-yl)cyclopentyl(1-methylcyclopropyl)carbamate (Intermediate 9)

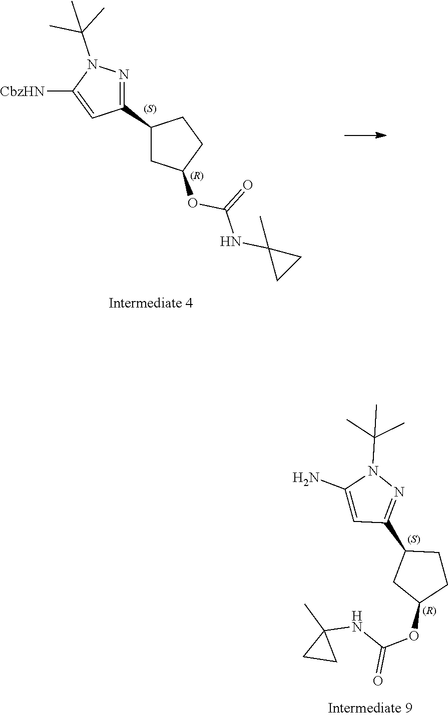

A mixture of Intermediate 4 2.1 g, 4.62 mmol) and Pd/C (10% 1.1 g) in EtOAc (40 mL) was degassed and purged with hydrogen (3 cycles) and stirred at rt for 16 h under a hydrogen balloon. The reaction mixture was filtered through celite and the filtrate evaporated under reduced pressure to afford the title compound as a pink solid (Intermediate 9, 1.4 g, 94%). MS (ES+) C₁₇H₂₈N₄O₂ requires: 320, found: 321 [M+H]⁺.

### Step 2. Synthesis of (1R,3S)-3-(1-(tert-butyl)-5-((3-chloro-1,2,4-triazin-5-yl)amino)-1H-pyrazol-3-yl)cyclopentyl(1-methylcyclopropyl)carbamate (Intermediate 10)

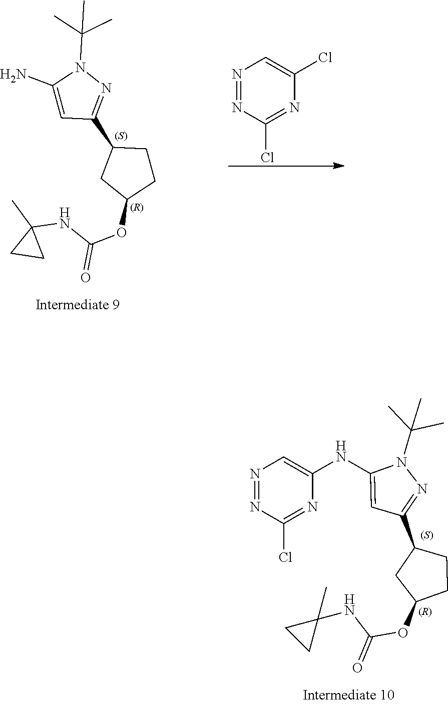

To a solution of Intermediate 9 (210 mg, 655 μmol) in dry THF (9 mL) was added LiHMDS (3.0 M in THF, 3 mL) at 0° C. and then stirred at 0° C. for 5 min. A solution of 3,5-dichloro-1,2,4-triazine (98.2 mg, 655 μmol) in dry-THF (1 mL) was added and the reaction mixture stirred at rt for 1 h. The reaction mixture was diluted with EtOAc and washed with water and brine. The combined organics were evaporated to dryness and the residue purified by flash chromatography (SiO₂, 50% EtOAc/PE) to afford the title compound as a yellow solid (36.5 mg, 12%). MS (ES+) C₂₀H₂₈ClN₇O₂ requires: 433, found: 434 [M+H]⁺.

### Step 3. Synthesis of (1R,3S)-3-(1-(tert-butyl)-5-((3-(methylamino)-1,2,4-triazin-5-yl)amino)-1H-pyrazol-3-yl)cyclopentyl(1-methylcyclopropyl)carbamate (Intermediate 11)

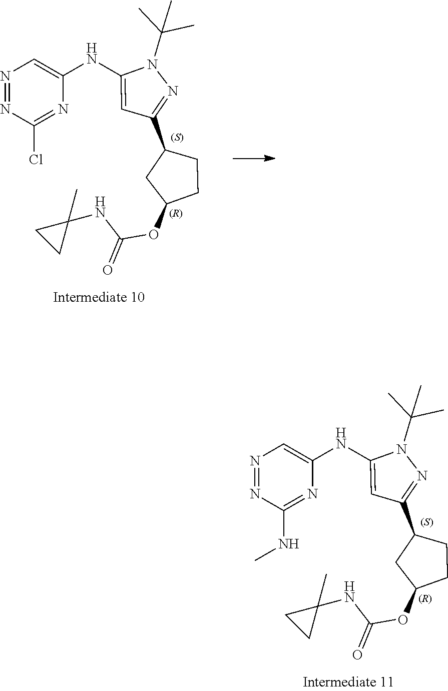

A mixture of Intermediate 10 (49 mg, 112 μmol), DIEA (72.3 mg, 560 μmol) and methylamine hydrochloride (46.7 mg, 560 μmol) in DMF (2 mL) was stirred at 90° C. for 2 h. The reaction mixture was diluted with EtOAc and washed with water and brine. The combined organics were evaporated to dryness and the residue was used in the next step directly. MS (ES+) C₂₁H₃₂N₈O₂ requires: 428, found: 429 [M+H]⁺.

### Step 4. Synthesis of (1R,3S)-3-(3-((3-(methylamino)-1,2,4-triazin-5-yl)amino)-1H-pyrazol-5-yl)cyclopentyl(1-methylcyclopropyl)carbamate (Example 23)

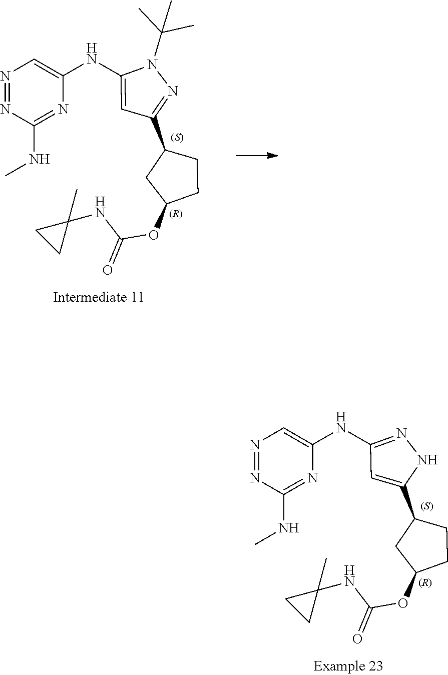

A solution of Intermediate 11 (50 mg, 116 μmol) in formic acid (2 mL) was stirred at 80° C. for 3 h. The mixture was evaporated to dryness in vacuo and the residue purified by prep-HPLC (Xtimate 150A, 21.2×250 mm, 10 mm; 15-95% MeCN/H₂O (0.1% NH₄HCO₃)) in 18 min) to obtain the title compound as a white solid (7.4 mg, 17%). MS (ES+) C₁₇H₂₄N₈O₂ requires: 372, found: 373 [M+H]⁺. ¹H-NMR (400 MHz, DMSO-d₆) δ ppm 12.15 (br. s., 1H), 10.06 (br. s, 1H), 8.20 (br. s, 1H), 7.34 (s, 1H), 6.92 (s, 1H), 6.47 (s, 1H), 4.99 (s, 1H), 3.07-3.03 (m, 1H), 282 (d, 3H, J=4.4 Hz), 2.50-2.40 (m, 1H), 2.10-1.96 (m, 1H), 1.96-1.84 (m, 1H), 1.84-1.64 (m, 2H), 1.64-1.51 (m, 1H), 1.22 (s, 3H), 0.58-0.46 (m, 4H).

### Example 24. (1R,3S)-3-(3-((6-methoxypyrazine-2-yl)amino)-1-H-pyrazol-5-yl)cyclopentyl(1-methylcyclopropyl)carbamate

### Step 1. Synthesis of (1R,3S)-3-(1-(tert-butyl)-5-((6-methoxypyrazine-2-yl)amino)-1H-pyrazol-3-yl)cyclopentyl(1-methylcyclopropyl)carbamate (Intermediate 12)

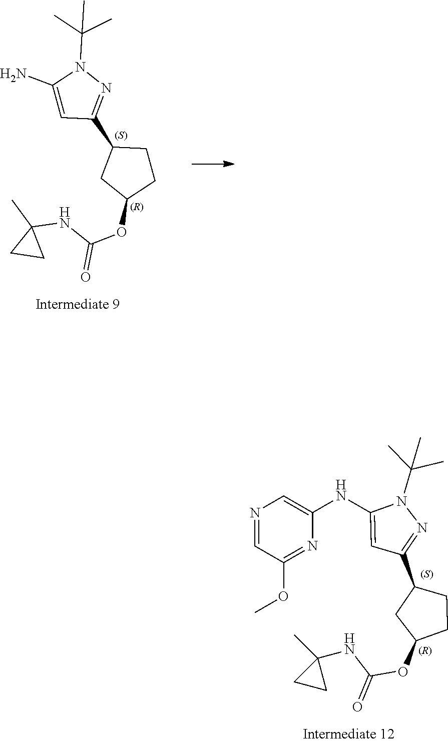

A mixture of Intermediate 9 (50.0 mg, 156 μmol), 2-chloro-6-methoxypyrazine (22.5 mg, 156 μmol), BrettPhos Pd G4 (28.7 mg, 31.2 μmol) and KOAc (61.1 mg, 624 μmol) in dioxane (1.5 mL) was stirred at 60° C. for 4 h under N₂. The reaction mixture was concentrated and the residue was by flash chromatography (SiO₂, 1:2 EtOAc/PE) to afford the title compound as a yellow solid (Intermediate 10, 40 mg, 59%). MS (ES+C₂₂H₃₂N₆O₃ requires: 428, found: 429 [M+H]⁺.

### Step 2. Synthesis of (1R,3S)-3-(1-(tert-butyl)-3-((6-methoxypyrazine-2-yl)amino)-1H-pyrazol-5-yl)cyclopentyl(1-methylcyclopropyl)carbamate (Example 24)

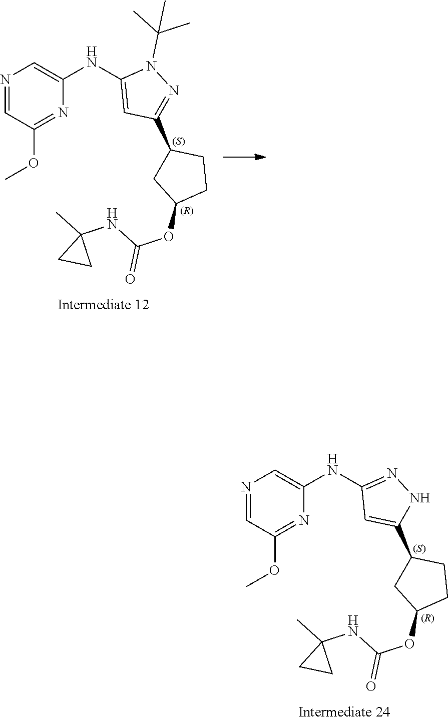

A solution of Intermediate 12 (40 mg, 93 μmol) in HCOOH (2 mL) was stirred at 80° C. for 14 h. The reaction mixture was evaporated and the residue purified by prep-HPLC (Xtimate 21.2×250 mm, 10 mm; 15-95% MeCN/H₂O (0.1% NH₄HCO₃)) to give the title compound as a yellow solid (16.8 mg, 48%). MS (ES+) C₁₈H₂₄N₆O₃ requires: 372, found: 373 [M+H]⁺. ¹H-NMR (400 MHz, DMSO-d₆) δ ppm 11.95 (br. s., 1H), 9.56 (s, 1H), 8.06 (s, 1H), 7.50 (s, 1H), 7.33 (s, 1H), 6.24 (s, 1H), 4.97 (s, 1H), 3.86 (s, 3H), 3.06-3.02 (m, 1H), 2.01-2.00 (m, 1H), 1.94-1.84 (s, 1H), 1.70-1.54 (m, 3H), 1.22 (s, 3H), 0.58-0.46 (m, 4H).

### Example 25-38

The title compounds were prepared from Intermediate 9 and the appropriate halo-heterocycle (Het) using an analogous 2-part procedure as described for Example 24

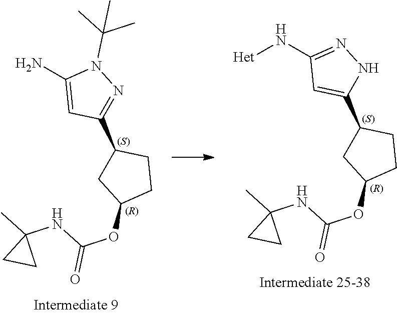

### Synthesis of 5-chloro-N,N-dimethylpyrazin-2-amine (Intermediate 13)

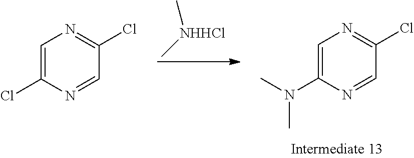

A mixture of 2,5-dichloropyrazine (500 mg, 3.36 mmol), dimethylamine hydrochloride (821 mg, 10.07 mmol) and Et₃N (1.30 g, 10.07 mmol) in NMP (5.0 mL) was stirred at 120° C. for 16 h. The reaction mixture was diluted with ethyl acetate and washed with water. The combined organics were evaporated to dryness and the residue was purified by silica gel chromatography (3:1 PE/EtOAc) to afford the title compound as a yellow solid (350 mg, 66% yield). MS (ES+) (C₆H₈ClN₃ requires: 157, found: 158 [M+H]⁺.

### Synthesis of 2-chloro-5-(methoxymethyl)pyrazine (Intermediate 14)

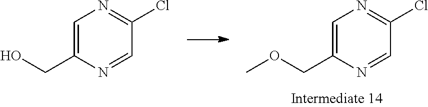

To a mixture of (5-chloropyrazin-2-yl)methanol (300 mg, 2.07 mmol) in dry THF (5 mL) was added NaH (120 mg, 5.00 mmol) at 0° C. and the mixture stirred for 30 ruin. MeI (442 mg, 3.11 mmol) was added and the reaction mixture stirred for 1 h, quenched with water and extracted with EtOAc. The combined organics were washed with brine, dried (Na₂SO₄) and evaporated to dryness in vacuo. The residue was purified by flash chromatography on silica gel (20% EtOAc/PE) to afford the title compound as a colourless oil (130 mg, 39% yield) as a colourless oil. H NMR (400 MHz, DMSO-d₆) δ ppm 8.77 (s, 1H), 8.52 (s, 1H), 4.58 (s, 2H), 3.39 (s, 3H).

| Number | Name, Structure, Reactants, Data |
|---|---|
| 25 | (1R,3S)-3-(3-((2-methoxypyrimidin-4-yl)amino)-1H-pyrazol-5- |
| | yl)cyclopentyl (1-methylcyclopropyl)carbamate |
| | 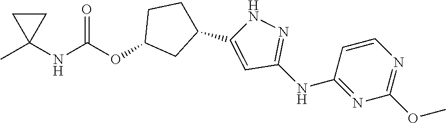 |
| | Het: 4-chloro-2-methoxypyrimidine |
| | Prep-HPLC (Xtimate, 21.2 × 250 mm, 10 mm; 15-95% MeCN/H₂O (0.1% |
| | NH₄HCO₃)); White solid (9.8 mg, 13% yield). |
| | MS (ES+) C₁₈H₂₄N₆O₃ requires: 372, found 373 [M + H]⁺. ¹H-NMR (400 |
| | MHz, DMSO-d₆) δ ppm 12.06 (br. s., 1H), 9.84 (s, 1H), 8.03 (d, J = 5.6 Hz, |
| | 1H), 7.33 (s, 1H), 6.71 (s, 1H), 6.28 (s, 1H), 4.98 (s, 1H) 3.82 (s, 3H), 3.06- |
| | 3.04 (m, 1H), 2.01-2.00 (m, 1H), 1.88-1.87 (m, 1H), 1.70-1.57 (m, 3H), |
| | 1.22 (s, 3H), 0.58-0.45 (m, 4H). |
| 26 | (1R,3S)-3-(3-(pyrazin-2-ylamino)-1H-pyrazol-5-yl)cyclopentyl (1- |
| | methylcyclopropyl)carbamate |
| | 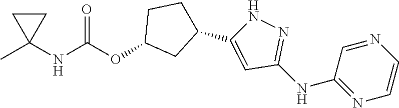 [SMILES: CC1(NC(=O)O[C@@H]2CC[C@H](c3cc(Nc4cnccn4)n[nH]3)C2)CC1] |
| | Het: 2-chloropyrazine |
| | Prep-HPLC (Xtimate, 21.2 × 250 mm, 10 mm; 15-95% MeCN/H₂O (0.1% |
| | NH₄HCO₃)); Yellow solid (3.5 mg, 8% yield). |
| | MS (ES+) C₁₇H₂₂N₆O₂ requires: 342, found: 343 [M + H]⁺. ¹H-NMR (400 |
| | MHz, DMSO-d₆) δ ppm 11.97 (br. s., 1H), 9.60 (s, 1H), 8.51 (s, 1H), 8.07 |
| | (s, 1H), 7.86 (d, J = 2.8 Hz, 1H), 7.36 (s, 1H), 6.20 (s, 1H), 4.97 (s, 1H), |
| | 3.06-3.04 (m, 1H), 2.00-1.99 (m, 1H), 1.88-1.87 (m, 1H), 1.71-1.68 (m, |
| | 2H), 1.68-1.57 (m, 1H), 1.34 (s, 5H), 0.58-0.46 (m, 4H). |
| 27 | (1R,3S)-3-(3-((5-methylpyrazin-2-yl)amino)-1H-pyrazol-5-yl)cyclopentyl |
| | (1-methylcyclopropyl)carbamate |
| | 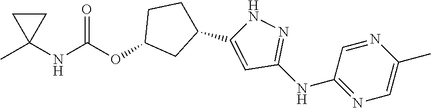 |
| | Het: 2-chloro-5-methylpyrazine |
| | Prep-HPLC (Xtimate, 21.2 × 250 mm, 10 mm; 15-95% MeCN/H₂O (0.1% |
| | NH₄HCO₃)); White solid (4.0 mg, 14% yield). |
| | MS (ES+) C₁₈H₂₄N₆O₂ requires: 356, found: 357 [M + H]⁺. ¹H-NMR (400 |
| | MHz, DMSO-d₆) δ ppm 11.90 (br. s., 1H), 9.36 (s, 1H), 8.48 (s, 1H), 7.97 |
| | (s, 1H), 7.36 (s, 1H), 6.12 (s, 1H), 4.96 (s, 1H), 3.04-3.03 (m, 1H), 2.32 (s, |
| | 3H), 1.99-1.97 (m, 2H), 1.70-1.68 (m, 2H), 1.58-1.57 (m, 1H), 1.44-1.42 (m, |
| | 1H), 1.23 (s, 3H), 0.59-0.57 (m, 2H), 0.46-0.44 (m, 2H). |
| 28 | (1R,3S)-3-(3-((5-methoxypyrazin-2-yl)amino)-1H-pyrazol-5-yl)cyclopentyl |
| | (1-methylcyclopropyl)carbamate |
| | 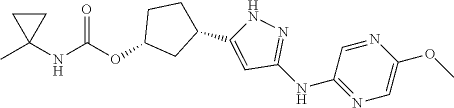 |
| | Het: 2-chloro-5-methoxypyrazine |
| | Prep-HPLC (Xtimate, 21.2 × 250 mm, 10 mm; 15-95% MeCN/H₂O (0.1% |
| | NH₄HCO₃)); Yellow solid (5.2 mg, 14% yield). |
| | MS (ES+) C₁₈H₂₄N₆O₃ requires: 372, found: 373 [M + H]⁺. ¹H-NMR (400 |
| | MHz, DMSO-d₆) δ ppm 11.79 (s, 1H), 9.09 (s, 1H), 8.34 (s, 1H), 7.88 (s, |
| | 1H), 7.36 (s, 1H), 5.93 (s, 1H), 4.98 (s, 1H), 3.81 (s, 3H), 3.03-3.00 (m, 1H), |
| | 2.08-1.94 (m, 1H), 1.94-1.82 (m, 2H), 1.78-1.63 (m, 2H), 1.63-1.51 (m, 1H), |
| | 1.23 (s, 3H), 0.59-0.58 (m, 2H), 0.47-0.46 (m, 2H). |
| 29 | (1R,3S)-3-(3-((6-cyclopropylpyrazin-2-yl)amino)-1H-pyrazol-5- |
| | yl)cyclopentyl (1-methylcyclopropyl)carbamate |
| | 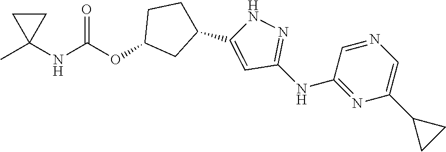 [SMILES: CC1(NC(=O)O[C@@H]2CC[C@H](c3cc(Nc4cncc(C5CC5)n4)n[nH]3)C2)CC1] |
| | Het: 2-chloro-6-cyclopropylpyrazine |
| | Prep-HPLC (Xtimate, 21.2 × 250 mm, 10 mm; 15-95% MeCN/H₂O (0.1% |
| | NH₄HCO₃)); Yellow solid (6.0 mg, 21.4% yield). |
| | MS (ES+) C₂₀H₂₆N₆O₂ requires: 382, found: 383 [M + H]⁺. ¹H-NMR (400 |
| | MHz, DMSO-d₆) δ ppm 11.93 (br. s., 1H), 9.47 (s, 1H), 8.21 (s, 1H), 7.85 |
| | (s, 1H), 7.35 (s, 1H), 6.18 (s, 1H), 4.99 (br. s., 1H), 3.31 (s, 3H), 3.03-3.00 |
| | (m, 1H), 2.02-1.99 (m, 4H), 1.70-1.68 (m, 1H), 0.96-0.92 (m, 4H), 0.87-0.84 |
| | (m, 1H), 0.59-0.45 (m, 4H). |
| 30 | (1R,3S)-3-(3-((5-(trifluoromethyl)pyrazin-2-yl)amino)-1H-pyrazol-5- |
| | yl)cyclopentyl (1-methylcyclopropyl)carbamate |
| | 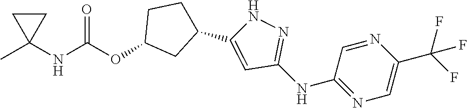 [SMILES: CC1(NC(=O)O[C@@H]2CC[C@H](c3cc(Nc4cnc(C(F)(F)F)cn4)n[nH]3)C2)CC1] |
| | Het: 2-chloro-5-(trifluoromethyl)pyrazine |
| | Prep-HPLC (Xtimate, 21.2 × 250 mm, 10 mm; 15-95% MeCN/H₂O (0.1% |
| | NH₄HCO₃)); Yellow solid (5.2 mg, 14% yield). |
| | MS (ES+) C₁₈H₂₁F₃N₆O₂ requires: 410, found: 411 [M + H]⁺. ¹H-NMR (400 |
| | MHz, DMSO-d₆) δ ppm 12.20 (br. s., 1H), 10.40 (s, 1H), 8.55 (s, 1H), 8.53 |
| | (s, 1H), 7.73 (s, 1H), 7.10-7.06 (m, 2H), 6.34 (s, 1H), 4.99 (s, 1H), 3.09-3.05 |
| | (m, 1H), 2.03-1.99 (m, 3H), 1.71-1.70 (m, 2H), 1.22 (s, 3H), 0.59-0.58 (m, |
| | 2H), 0.47-0.46 (m, 2H). |
| 31 | (1R,3S)-3-(3-((5-fluoropyrazin-2-yl)amino)-1H-pyrazol-5-yl)cyclopentyl (1- |
| | methylcyclopropyl)carbamate |
| | 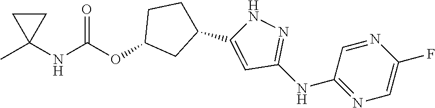 [SMILES: CC1(NC(=O)O[C@@H]2CC[C@H](c3cc(Nc4cnc(F)cn4)n[nH]3)C2)CC1] |
| | Het: 2-chloro-5-fluoropyrazine |
| | Prep-HPLC (Xtimate, 21.2 × 250 mm, 10 mm; 15-95% MeCN/H₂O (0.1% |
| | NH₄HCO₃)); White solid (3.2 mg, 14% yield). |
| | MS (ES+) C₁₇H₂₁FN₆O₂ requires: 360, found: 361 [M + H]⁺. ¹H-NMR (400 |
| | MHz, DMSO-d₆) δ ppm 11.95 (br, s., 1H), 9.64 (s, 1H), 8.26 (s, 1H), 8.16 |
| | (d, J = 8.0 Hz, 1H), 7.36 (s, 1H), 6.06 (s, 1H), 4.98 (s, 1H), 3.08-3.06 (m, |
| | 1H), 2.02-1.67 (m, 6H), 1.23 (s, 3H), 0.60-0.58 (m, 2H), 0.48-0.47 (m, 2H). |
| 32 | (1R,3S)-3-(3-((5-(dimethylamino)pyrazin-2-yl)amino)-1H-pyrazol-5- |
| | yl)cyclopentyl (1-methylcyclopropyl)carbamate |
| | 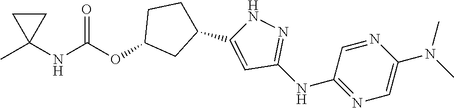 |
| | Het: 5-chloro-N,N-dimethylpyrazin-2-amine (Intermediate 13) |
| | Prep-HPLC (Xtimate, 21.2 × 250 mm, 10 mm; 15-95% MeCN/H₂O (0.1% |
| | NH₄HCO₃)); Yellow solid (1.5 mg, 43% yield). |
| | MS (ES+) C₁₉H₂₇N₇O₂ requires: 385, found: 386 [M + H]+. 1H-NMR (500 |
| | MHz, DMSO-d6) δ ppm 11.68 (br. s., 1H), 8.70 (s, 1H), 8.33 (s, 1H), 7.72 |
| | (s, 1H), 7.34 (s, 1H), 5.90 (s, 1H), 4.97 (s, 1H), 3.07-2.97 (m, 1H), 2.94 (s, |
| | 6H), 2.47-2.41 (m, 1H), 2.02-1.96 (m, 1H), 1.91-1.83 (m, 1H), 1.73-1.64 |
| | (m, 2H), 1.58-1.52 (m, 1H), 1.23 (s, 3H), 0.59-0.47 (m, 4H). |
| 33 | (1R,3S)-3-(3-((2-(difluoromethyl)pyrimidin-4-yl)amino)-1H-pyrazol-5- |
| | yl)cyclopentyl (1-methylcyclopropyl)carbamate |
| | 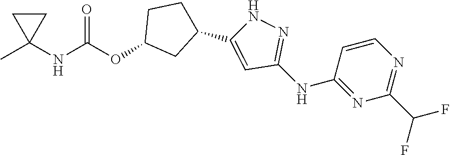 [SMILES: CC1(NC(=O)O[C@@H]2CC[C@H](c3cc(Nc4ccnc(C(F)F)n4)n[nH]3)C2)CC1] |
| | Het: 4-chloro-2-(difluoromethyl)pyrimidine |
| | Prep-HPLC (Xtimate, 21.2 × 250 mm, 10 mm; 15-95% MeCN/H₂O (0.1% |
| | NH₄HCO₃)); Yellow solid (31.4 mg, 39% yield). |
| | MS (ES+) C₁₈H₂₂F₂N₆O₃ requires: 392, found: 393 [M + H]⁺. ¹H-NMR (400 |
| | MHz, DMSO-d₆) δ ppm 12.18 (br. S, 1H), 10.25 (s, 1H), 8.37 (s, 1H), 7.35 |
| | (s, 1H), 6.71 (t, J = 54.4 Hz, 1H), 4.98 (s 1H), 3.09-3.02 (m, 1H), 2.05-2.00 |
| | (m, 2H), 1.91-1.85 (m, 2H), 1.73-1.58 (m, 2H), 1.23 (m, 3H), 0.59-0.57 (m, |
| | 2H), 0.48-0.46 (m, 2H). |
| 34 | (1R,3S)-3-(3-((2-methylpyrimidin-4-yl)amino)-1H-pyrazol-5-yl)cyclopentyl |
| | (1-methylcyclopropyl)carbamate |
| | 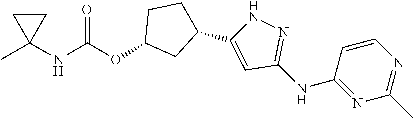 |
| | Het: 4-chloro-2-methylpyrimidine |
| | Prep-HPLC (Xtimate, 21.2 × 250 mm, 10 mm; 15-95% MeCN/H₂O (0.1% |
| | NH₄HCO₃)); White solid (33.4 mg, 39% yield). |
| | MS (ES+) C₁₈H₂₄N₆O₂ requires: 356, found: 357 [M + H]⁺. ¹H NMR (500 |
| | MHz, DMSO-d₆) δ ppm 12.00 (br. s., 1H), 9.66 (s, 1H), 8.14 (d, J = 4.8 Hz, |
| | 1H), 7.34 (s, 1H), 7.09 (s, 1H), 6.07 (s, 1H), 4.98 (s, 1H), 3.05-3.02 (m, |
| | 1H), 2.43 (s, 3H), 2.02-2.00 (m, 1H), 1.90-1.86 (m, 1H), 1.70-1.57 (m, 3H), |
| | 1.23 (s, 3H), 0.59-0.46 (m, 4H). |
| 35 | (1R,3S)-3-(3-((5-(methylsulfonyl)pyrazin-2-yl)amino)-1H-pyrazol-5- |
| | yl)cyclopentyl (1-methylcyclopropyl)carbamate |
| | 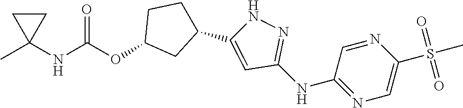 |
| | Het: 2-bromo-5-methanesulfonylpyrazine |
| | Prep-HPLC (Xtimate, 21.2 × 250 mm, 10 mm; 15-95% MeCN/H₂O (0.1% |
| | NH₄HCO₃)); Yellow solid 21.5 mg, 31% yield). |
| | MS (ES+) C₁₈H₂₄N₆O₄S requires: 420, found: 421 [M + H]⁺. ¹H-NMR (500 |
| | MHz, DMSO-d₆) δ ppm 12.23 (br s, 1H), 10.59 (s, 1H), 8.62 (s, 1H), 8.51 |
| | (s, 1H), 7.35 (s, 1H), 6.34 (s, 1H), 4.99 (s, 1H), 3.18 (s, 3H), 3.11-3.04 (m, |
| | 1H), 2.48-2.45 (m, 1H), 2.09-1.96 (m, 1H), 1.96-1.84 (m, 1H), 1.81-1.65 (m, |
| | 2H), 1.65-1.53 (m, 1H), 1.23 (s, 3H), 0.60-0.47 (m, 4H). |
| 36 | (1R,3S)-3-(3-((5-(difluoromethyl)pyrazin-2-yl)amino)-1H-pyrazol-5- |
| | yl)cyclopentyl (1-methylcyclopropyl)carbamate |
| | 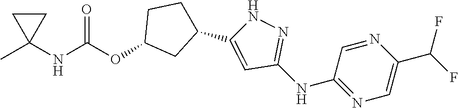 [SMILES: CC1(NC(=O)O[C@@H]2CC[C@H](c3cc(Nc4cnc(C(F)F)cn4)n[nH]3)C2)CC1] |
| | Het: 2-chloro-5-(difluoromethyl)pyrazine |
| | Prep-HPLC (Xtimate, 21.2 × 250 mm, 10 mm; 15-95% MeCN/H₂O (0.1% |
| | NH₄HCO₃)); White solid (33.3 mg, 54% yield). |
| | MS (ES+) C₁₈H₂₂F₂N₆O₂ requires: 392, found: 393 [M + H]⁺. ¹H-NMR (400 |
| | MHz, DMSO-d₆) δ ppm 10.12 (br. S, 1H), 8.52 (s, 1H), 8.36 (s, 1H), 7.42 (s, |
| | 1H), 6.92 (t, J = 55.2 Hz, 1H), 6.26 (s, 1H), 4.98 (s, 1H), 3.04 (m, 1H), 2.49- |
| | 2.07 (m, 2H), 1.85-1.70 (m, 2H), 1.69-1.64 (m, 2H), 1.23 (s, 3H), 0.58-0.57 |
| | (m, 2H), 0.47-0.46 (m, 2H). |
| 37 | (1R,3S)-3-(3-((5-(methoxymethyl)pyrazin-2-yl)amino)-1H-pyrazol-5- |
| | yl)cyclopentyl (1-methylcyclopropyl)carbamate |
| | 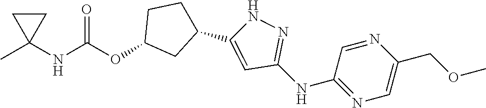 |
| | Het: 2-chloro-5-(methoxymethyl)pyrazine (Intermediate 14) |
| | Prep-HPLC (Boston pHlex ODS, 21.2 × 250 mm, 10 mm; 30-55% |
| | MeCN/H₂O (0.1% HCO₂H)); Yellow solid (21 mg, 12% yield). |
| | MS (ES+) C₁₉H₂₆N₆O₃ requires: 386, found: 387 [M + H]⁺. ¹H-NMR (400 |
| | MHz, DMSO-d₆) δ ppm 11.99 (br. s, 1H), 9.63 (s, 1H), 8.51 (s, 1H), 8.10 (s, |
| | 1H), 7.36 (s, 1H), 6.17 (s, 1H), 4.99 (s, 1H), 4.36 (s, 2H), 2.51 (s, 3H), 3.06- |
| | 2.98 (m, 1H), 2.12-2.00 (m, 1H), 2.00-1.90 (m, 1H), 1.90-1.75 (m, 2H), |
| | 1.75-1.60 (m, 1H), 1.22 (s, 3H), 0.59-0.57 (m, 2H), 0.49-0.47 (m, 2H). |
| 38 | (1R,3S)-3-(3-(pyridazin-3-ylamino)-1H-pyrazol-5-yl)cyclopentyl (1- |
| | methylcyclopropyl)carbamate |
| | 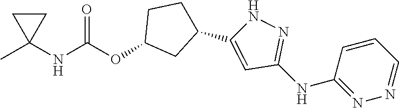 [SMILES: CC1(NC(=O)O[C@@H]2CC[C@H](c3cc(Nc4cccnn4)n[nH]3)C2)CC1] |
| | Het: 3-chloropyridazine |
| | Prep-HPLC (Boston pHlex ODS, 21.2 × 250 mm, 10 mm; 15-35% |
| | MeCN/H₂O (0.1% HCO₂H)); Yellow solid (8.8 mg, 14% yield). |
| | MS (ES+) C₁₇H₂₂N₆O₂ requires: 342, found: 343 [M + H]⁺. ¹H-NMR (400 |
| | MHz, DMSO-d₆) δ ppm 11.91 (br. s, 1H), 9.53 (s, 1H), 8.59 (d, J = 4.4 Hz, |
| | 1H), 7.51 (d, J = 9.2 Hz, 1H), 7.53-7.37 (m, 2H), 6.19 (s, 1H), 5.00 (s, 2H), |
| | 3.09-3.03 (m, 2H), 2.02-2.00 (m, 1H), 1.91-1.89 (m, 1H), 1.72-1.70 (m. 2H), |
| | 1.68-1.58 (m, 1H), 1.23 (s, 3H), 0.59-0.58 (m, 2H), 0.47-0.46 (m, 2H). |

### Example 39. (1R,3S)-3-(3-(pyrimidin-4-ylamino)-1H-pyrazol-5-yl)cyclopentyl(1-methylcyclopropyl)carbamate

### Step 1. Synthesis of (1R,3S)-3-(1-(tert-butyl)-5-(pyrimidin-4-ylamino)-1H-pyrazol-3-yl)cyclopentyl(1-methylcyclopropyl)carbamate (Intermediate 15)

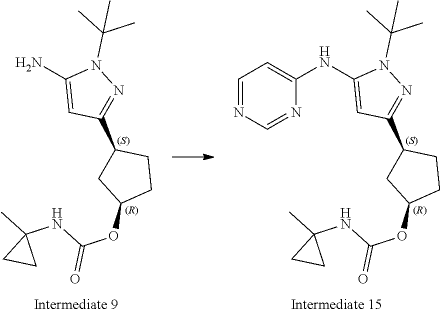

To a mixture of Intermediate 9 (60 mg, 187 μmol) in dry THE (3 mL) was added LHMDS (1M in THF, 1.0 mL, 1.0 mmol) at 0° C. and the mixture stirred for 30 min. A solution of 4-chloropyrimidine (21.4 mg, 187 μmol) in dry THF (0.5 mL) was added and the reaction mixture stirred at 0° C. for 3 h under nitrogen. The reaction mixture was diluted with EtOAc and washed with water and the organic layer evaporated to dryness in vacuo. The residue was purified by flash chromatography on silica gel (33% EtOAc/PE) to afford the title compound as a yellow solid (40 mg, 54% yield) MS (ES+) C₂₁H₃₀N₆O₂ requires: 398, found: 399 [M+H]⁺.

### Step 2. Synthesis of (1R,3S-3-(3-(pyrimidin-4-ylamino)-1H-pyrazol-5-yl)cyclopentyl(1-methylcyclopropyl)carbamate (Example 39)

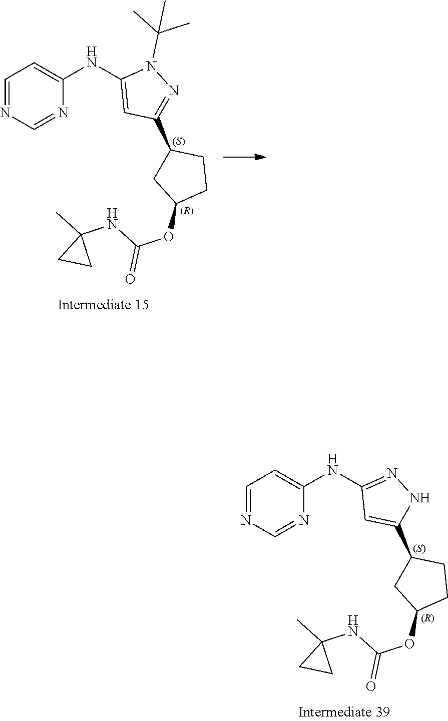

A solution of Intermediate 15 (40 mg, 100 μmol) in formic acid (5 mL) was stirred at 80° C. for 3 h. The reaction mixture was evaporated to dryness in vacuo and the residue was purified by Prep-HPLC (Xtimate, 21.2×250 mm, 10 mm; 15-95% MeCN/H₂O (0.1% NH₄HCO₃)) to afford the title product as a yellow solid (5.0 mg, 14% yield). MS (ES+) (C₁₇H₂₂N₆O₂ requires: 342, found: 343 [M+H]⁺. 1H-NMR (400 MHz, DMSO-d₆) δ ppm 12.07 (br. s., 1H), 9.79 (s, 1H), 8.55 (s, 1H), 8.32 (s, 1H), 8.23 (d, J=5.6 Hz, 1H), 7.35 (s, 1H), 7.20 (s, 1H), 6.17 (s, 1H), 4.98 (s, 1H), 3.06-3.03 (m, 2H), 2.03-1.63 (m, 6H), 1.23 (s, 3H), 0.61-0.56 (m, 2H), 0.48-0.46 (m, 2H).

### Example 40-42

The title compounds were prepared from (Intermediate 9) and the appropriate halo-heterocycle (Het) using an analogous 2-part procedure as described for Example 39.

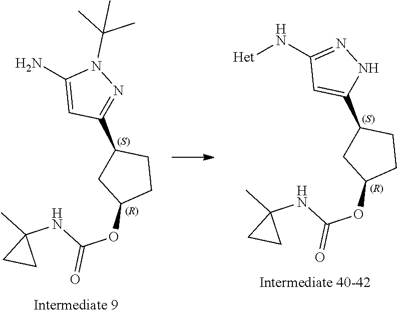

| Num- | |
|---|---|
| ber | Name, Structure, Reactants, Data |
| 40 | (1R,3S)-3-(3-((5-methylpyrimidin-4-yl)amino)-1H-pyrazol-5-yl) |
| | cyclopentyl(1-methylcyclopropyl)carbamate |
| | 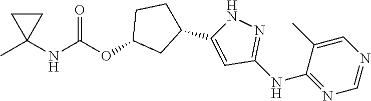 [SMILES: Cc1cncnc1Nc1cc([C@H]2CC[C@@H](OC(=O)NC3(C)CC3)C2)[nH]n1] |
| | Het: 4-chloro-5-methylpyrimidine |
| | Prep-HPLC (Welch, 21.2 × 250 mm, 10 mm; 55% MeCN/H₂O |
| | (0.1% HCO₂H)); White solid (4.0 mg, 18% yield). |
| | MS (ES+) C₁₈H₂₄N₆O₂ requires: 356, found: 357 [M + H]⁺. |
| | ¹H-NMR (400 MHz, DMSO-d₆) δ ppm 12.11 (br. s., 1H), 8.82 |
| | (s, 1H), 8.44 (s, 1H), 8.08 (s, 1H), 7.35 (s, 1H), 6.39 (s, 1H), |
| | 4.99 (s, 1H), 3.06-3.04 (m, 1H), 2.14 (s, 3H), 2.03-1.86 (m, 2H), |
| | 1.75-1.72 (m, 2H), 1.62-1.60 (m, 1H), 1.23 (s, 3H), |
| | 0.60-0.58 (m, 2H), 0.47-0.45 (m, 2H). |
| 41 | (1R,3S)-3-(3-((5-chloropyrazin-2-yl)amino)-1H-pyrazol-5-yl) |
| | cyclopentyl(1-methylcyclopropyl)carbamate |
| | 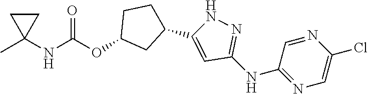 [SMILES: CC1(NC(=O)O[C@@H]2CC[C@H](c3cc(Nc4cnc(Cl)cn4)n[nH]3)C2)CC1] |
| | Het: 2,5-dichloropyrazine |
| | Prep-HPLC (Xtimate, 21.2 × 250 mm, 10 mm; 15-95% MeCN/ |
| | H₂O (0.1% NH₄CO₃)); Yellow solid (5.0 mg, 19% yield). |
| | MS (ES+) C₁₇H₂₁ClN₆O₂ requires: 376 found: 377 [M + H]⁺. |
| | 1H-NMR (400 MHz, DMSO-d6) δ ppm 12.16 (br. s., 1H), |
| | 8.81 (s, 1H), 8.13 (s, 1H), 7.77 |
| | (s, 1H), 7.35 (s, 1H), 6.32 (s, 1H), 4.99 (s, 1H), 3.09-3.05 (m, 1H), |
| | 2.03-1.61 (m, 6H), 1.23 (s, 3H), 0.61-0.47 (m, 4H). |
| 42 | (1R,3S)-3-(3-((1,2,4-triazin-3-yl)amino)-1H-pyrazol-5-yl) |
| | cyclopentyl (1-methylcyclopropyl)carbamate |
| | 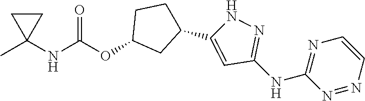 [SMILES: CC1(NC(=O)O[C@@H]2CC[C@H](c3cc(Nc4nccnn4)n[nH]3)C2)CC1] |
| | Het: 3-chloro-1,2,4-triazine |
| | Prep-HPLC (Xtimate, 21.2 × 250 mm, 10 mm; 15-95% |
| | MeCN/H₂O (0.1% NH₄HCO₃)); Yellow solid (2.8 mg, 8% yield). |
| | MS (ES+) C₁₆H₂₁N₇O₂ requires: 343, found: 344 [M + H]⁺. |
| | ¹H-NMR (400 MHz, DMSO-d₆) δ ppm 10.18 (br. s., 1H), |
| | 8.75 (s, 1H), 8.40 (s, 1H), 7.36 (s, 1H), 6.35 (s, 1H), 5.00 |
| | (s, 1H), 3.23-3.21 (m, 1H), 2.03-1.99 (m, 2H), 1.72-1.70 (m, 2H), |
| | 1.46-1.45 (m, 2H), 1.22 (s, 3H), 0.58-0.57 (m, 2H), 0.47- |
| | 0.45 (m, 2H). |

### Example 43 (1R,3S)-3-(3-((5-cyano-6-(methylamino)pyrazin-2-yl)amino)-1H-pyrazol-5-yl)cyclopentyl(1-methylcyclopropyl)carbamate

### Step 1. Synthesis of (1R,3S)-3-(1-(tert-butyl)-5-((6-chloro-5-cyanopyrazin-2-yl)amino)-1H-pyrazol-3-yl)cyclopentyl(1-methylcyclopropyl)carbamate (Intermediate 16)

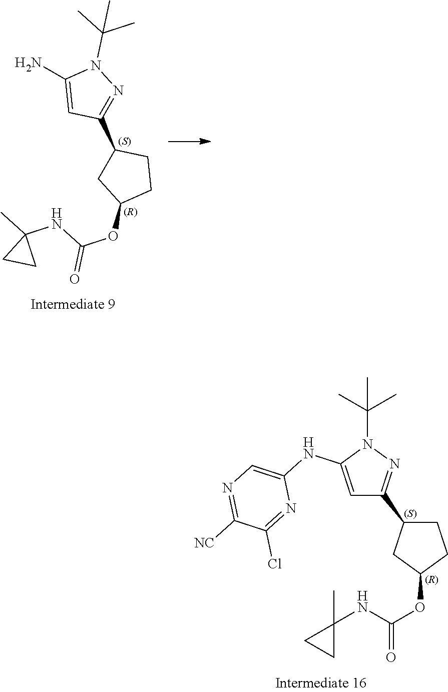

To a mixture of Intermediate 9 (95 mg, 296 μmol) in dry THF (9 mL) was added LHMDS (3.0 M in THF, 3 mL, 9.0 mmol) at 0° C. and the mixture was stirred at 0° C. for 20 min. To this was added a solution of 3,5-dichloropyrazine-2-carbonitrile (51.4 mg, 296 μmol) in dry THF (1 mL) and the reaction mixture stirred at rt for 1 h. The reaction mixture was diluted with EtOAc and washed with water and brine. The combined organics were evaporated to dryness and the residue was purified by flash chromatography on silica gel (3:2 PE/EtOAc) to afford the title compound as a yellow solid (50 mg, 37%) yield). MS (ES+) C₂₂H₂₈ClN₇O₂ requires: 457, found: 458 [M+H]⁺.

### Step 2. Synthesis of (1R,3S)-3-(1-(tert-butyl)-5-((5-cyano-6-(methylamino)pyrazin-2-yl)amino)-1H-pyrazol-3-yl)cyclopentyl(1-methylcyclopropyl)carbamate (Intermediate 17)

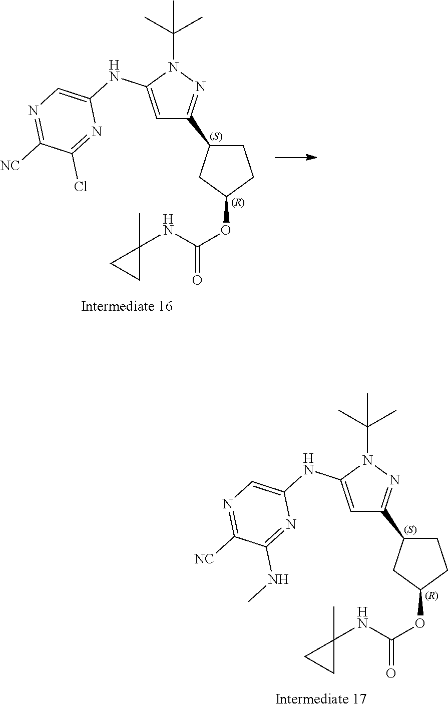

A mixture of Intermediate 16 (50 mg, 109 μmol), DIPEA (70.3 mg, 544 μmol) and methylamine hydrochloride (72.9 mg, 1.08 mmol) in DMF (5 mL) was stirred at 90° C. for 2 h. The reaction mixture was diluted with EtOAc and washed with water and brine. The organic layer was evaporated to dryness in vacuo to afford the title compound which was used without further purification. MS (ES+) C₂H₃₂N₈O₂ requires: 452, found: 453 [M+H]⁺.

### Step 3. Synthesis of (1R,3S)-3-(3-((5-cyano-6-(methylamino)pyrazin-2-yl)amino)-1H-pyrazol-5-yl)cyclopentyl(1-methylcyclopropyl)carbamate (Example 43)

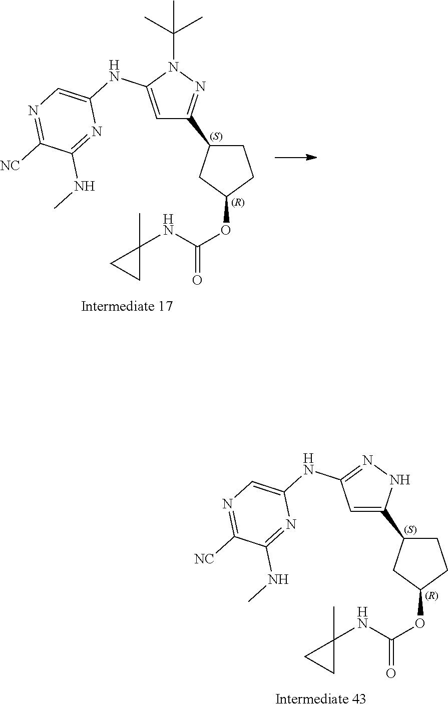

A solution of Intermediate 17 (45 mg, 99.4 μmol) in formic acid (4 mL) was stirred at 80° C. for 3 h. The mixture was evaporated to dryness in vacuo and the residue was purified with prep-HPLC (Xtimate, 21.2×250 mm, 10 mm; 15-95% MeCN/H₂O (0.1% NH₄HCO₃)) to obtain the title product as a white solid (4.3 mg, 11% yield). MS (ES+) C₁₉H₂₄N₈O₂ requires: 396, found: 397 [M+H]⁺. 1H-NMR (400 MHz, DM₆SO-d₆) δ ppm 12.15 (br. s., 1H), 10.25 (s, 1H), 7.56 (s, 1H), 7.33 (s, 1H), 7.16 (d, J=4.4 Hz, 1H), 6.49 (s, 1H) 4.98 (s, 1H), 3.10-3.03 (m, 1H), 2.87 (d, J=4.4 Hz, 3H), 2.50-2.44 (m, 1H), 2.04-1.98 (m 1H), 1.94-1.85 (m, 1H), 1.76-1.64 (m, 2H), 1.60-1.54 (m, 1H), 1.23 (s, 3H), 0.58-0.46 (m, 4H).

### Example 44 (1R,3S)-3-(3-((3-(dimethylamino)-1,2,4-triazin-5-yl)amino)-1H-pyrazol-5-yl)cyclopentyl(1-methylcyclopropyl)carbamate

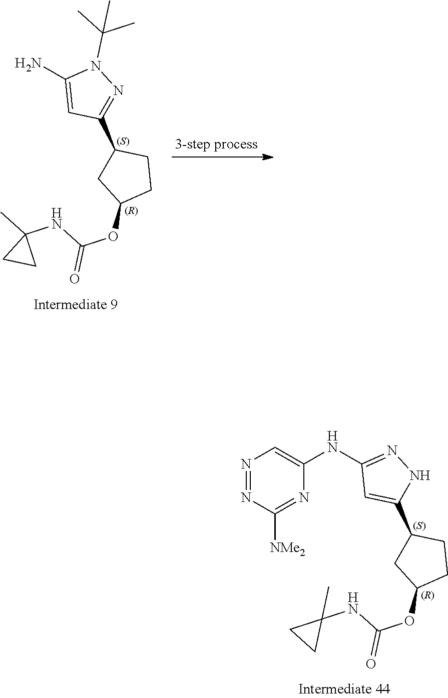

The title compound was prepared as a white solid (6.1 mg, 17% yield, final step) from Intermediate 9 using an analogous 3-part method to that described for Example 43. MS (ES+) C₁₈H₂₆N₈O₂ requires: 386, found: 387 [M+H]⁺. ¹H-NMR (400 MHz, DMSO-d₆) δ ppm 12.18 (br. s., 1H), 10.14 (s, 1H), 8.18 (s, 1H), 7.33 (s, 1H), 6.45 (s, 1H), 4.98 (s, 1H), 3.12 (s, 6H), 3.07-3.02 (m, 1H), 2.48-2.44 (m, 1H), 2.09-1.96 (m, 1H), 1.96-1.85 (m, 1H), 1.85-1.63 (m, 2H), 1.63-1.51 (m, 1H), 1.22 (s, 3H), 0.57-0.46 (m, 4H).

### Example 45 (1R,3S)-3-(3-((5-methoxy-3-methylpyrazin-2-yl)amino)-1H-pyrazol-5-yl)cyclopentyl(1-methylcyclopropyl)carbamate

### Step 1. Synthesis of (1R,3S)-3-(3-amino-1-tosyl-1H-pyrazol-5-yl)cyclopentyl(1-methylcyclopropyl)carbamate or (1R,3S)-3-(5-amino-1-tosyl-1H-pyrazol-3-yl)cyclopentyl(1-methylcyclopropyl)carbamate (intermediate 18)

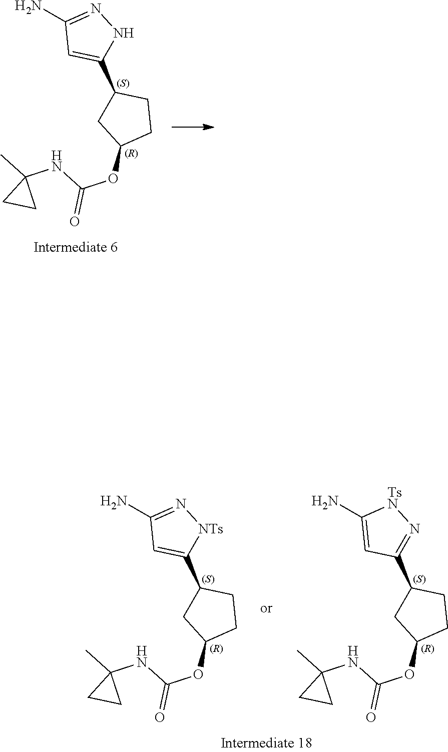

A mixture of Intermediate 6 (300 mg, 1.13 mmol), TsCl (215 mg, 1.13 mmol) and DIPEA (436 mg, 3.38 mmol) in DCM (3 mL) was stirred at room temperature for 1 h. The reaction mixture was diluted with EtOAc and washed with water. The combined organics were concentrated in vacuum and the residue purified by flash chromatography on silica gel (50% PE/EtOAc) to afford the title compound as a colourless oil (173 mg, 36% yield). MS (ES+) C₂₀H₂₆N₄O₄S requires: 418, found: 419 [M+H]⁺.

### Step 2. Synthesis of (1R,3S)-3-(3-((6-methoxy-3-methylpyrazin-2-yl)amino)-1-tosyl-1H-pyrazol-5-yl)cyclopentyl(1-methylcyclopropyl)carbamate or (1R,3S)-3-(5-((5-methoxy-3-methylpyrazin-2-yl)amino)-1-tosyl-1H-pyrazol-3-yl)cyclopentyl(1-methylcyclopropyl)carbamate (Intermediate 19)

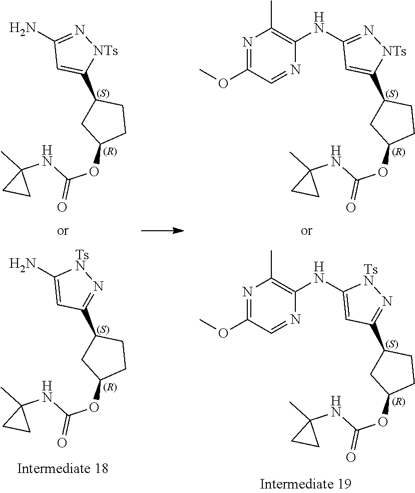

A mixture of Intermediate 18 (173 mg, 413 μmol), 2-bromo-5-methoxy-3-methylpyrazine (84 mg, 413 μmol), KOAc (120 mg, 1.23 mmol) and BrettPhos Pd G4 (38 mg, 41.3 μmol) was stirred at 100° C. overnight under N₂. The residue was purified via flash chromatography on silica gel (66% PE/EtOAc) to afford the title compound as a colourless oil (122 mg, 54% yield). MS (ES+) C₂₆H₃₂N₆O₈S requires: 540, found: 541[M+H]⁺.

### Step 6. Synthesis of (1R,3S)-3-(3-((5-methoxy-3-methylpyrazin-2-yl)amino)-1H-pyrazol-5-yl)cyclopentyl(1-methylcyclopropyl)carbamate (Example 45)

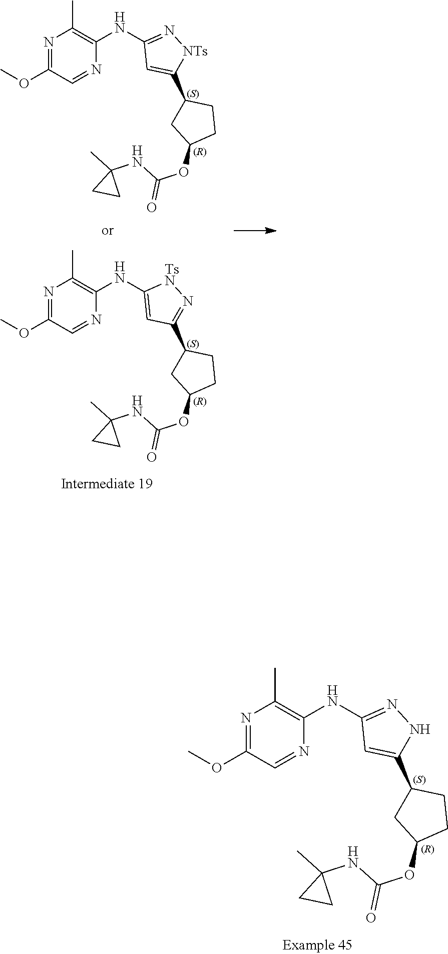

A mixture of Intermediate 19 (120 mg, 221 μmol) and potassium hydroxide (30 mg, 534 μmol) in ethanol (5 mL) was stirred at 65° C. for 1 h. The reaction mixture was neutralized by addition of 4M HCl/Dioxane and evaporated to dryness in vacuo and the residue purified by prep-1-HPLC (Welch, 21.2×250 mm, 10 mm; 5-95% MeCN/H₂O (0.1% 10 mM NH₄HCO₃+NH₄OH) to afford the title compound as a white solid (29.8 mg, 34% yield). MS (ES+) C₁₉H₂₆N₆O₃ requires: 386, found: 387 [M+H]⁺. ¹H-NMR (400 MHz, DMSO-d₆) δ ppm 11.84 (br. s, 1H), 8.23 (s, 1H), 7.70 (s, 1H), 7.35 (s, 1H), 6.14 (s, 1H), 4.98 (s, 1H), 3.80 (s, 3H), 3.04-3.03 (m, 1H), 2.45-2.41 (m, 1H), 2.40 (s, 3H), 2.03-1.99 (m, 1H), 1.90-1.88 (m, 1H), 1.62-1.59 (m, 2H), 1.58-1.52 (m, 1H), 1.23 (s, 3H), 0.59-0.56 (m, 2H), 0.48-0.46 (m, 2H).

### Example 46 (1R,3S)-3-(3-((2-(hydroxymethyl)pyrimidin-4-yl)amino)-1-pyrazol-5-yl)cyclopentyl(1-methylcyclopropyl)carbamate

### Step 1. Synthesis of methyl 4-((5-((1 S3R)-3-(((1-methylcyclopropyl)carbamoyl)oxy)cyclopent-yl)-1H-pyrazol-3-yl)amino)pyrimidine-2-carboxylate (Intermediate 20)

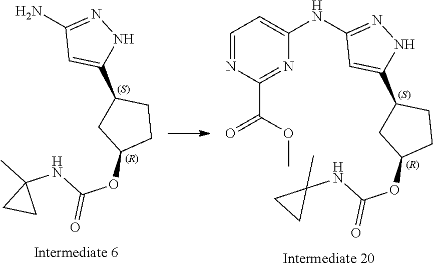

A mixture of methyl 4-chloropyrimidine-2-carboxylate (168 mg, 973 μmol), Intermediate 6 (282 mg, 1.07 mmol) and DIPEA (0.48 mL, 2.92 mmol) in DMSO (4 mL) was heated at 130° C. for 18 hrs under N₂. The cooled mixture was filtered and the filtrate diluted with water at 0° C. and extracted with EtOAc. The combined organics were evaporated to dryness in vacuo and the residue purified by silica gel chromatography 10:1 DCM/MeOH) to give the title compound (120 mg, 31% yield). MS (ES+) C₁₉H₂₄N₆O₄ requires: 400, found: 401[M+H]⁺.

### Step 2. Synthesis of (1R,3S)-3-(3-((2-(hydroxymethyl)pyrimidin-4-yl)amino)-1H-pyrazol-5-yl)cyclopentyl(1-methylcyclopropyl)carbamate (Example 51)

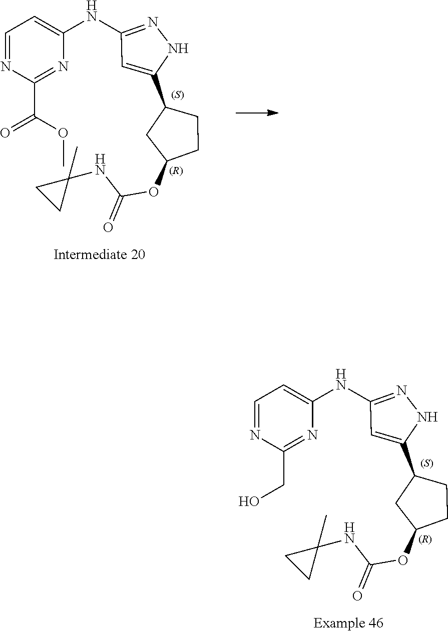

LAH (100 mg, 2.62 mmol) was added at 0° C. to a mixture of Intermediate 20 (309 mg, 771 μmol) in THE (5 mL) and the mixture stirred at 0° C. for 30 min. Water (0.1 mL), 10% NaOH (0.1 mL) and water (0.3 mL) were added sequentially at 0° C. and the resulting mixture filtered. The filtrate was evaporated to dryness in vacuo and the residue purified by prep-HPLC (Xtimate, 21.2×250 mm, 10 mm; 5-95% MeCN/H₂O (0.1% NH₄HCO₃) to give the title compound as a white solid (10.3 mg, 4% yield). MS (ES+) C₁₈H₂₄N₆O₃ requires: 372, found: 373 [M+H]⁺. ¹H NMR (500 MHz, DMSO-d₆) δ 12.04 (br s, 1H) 9.83 (br.s., 1H), 8.24 (d, J=5.5 Hz, 1H) 7.33 (s, 1H), 7.09 (br.s., 1H), 6.13 (br.s., 1H), 4.98-4.93 (m, 2H), 4.42 (d, J=5.5 Hz, 2H), 3.06-3.03 (m, 1H), 2.45-2.42 (m, 1H), 2.05-1.58 (m, 5H), 1.23 (s, 3H), 0.59-0.45 (m, 4H).

### Example 47 (1R,3S)-3-(3-((5-(hydroxymethyl)-3-methylpyrazin-2-yl)amino)-1H-pyrazol-5-yl)cyclopentyl(1-methylcyclopropyl)carbamate

### Step 1. Synthesis of methyl 6-methyl-5-((5-((1S,3R)-3-(((1-methylcyclopropyl)carbamoyl)oxy)cyclopentyl-1H-pyrazol-3-yl)amino)pyrazine-2-carboxylate (Intermediate 21)

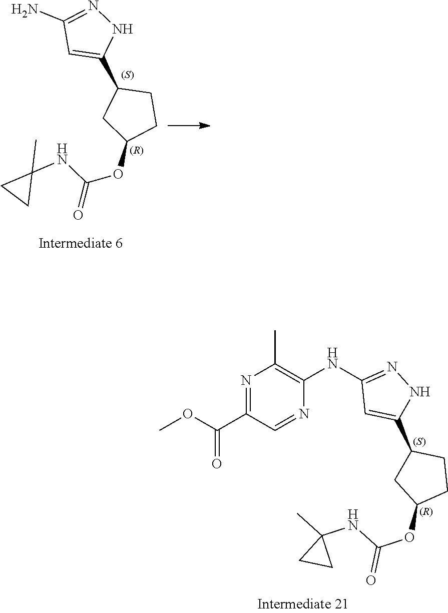

A mixture of Intermediate 6 (212 mg, 803 μmol), methyl 5-chloro-6-methylpyrazine-2-carboxylate (150 mg, 803 μmol), BrettPhos Pd G₄ (74 mg, 80.3 μmol) and KOAc (235 mug, 2.40 mmol) in dioxane (10 mL) was stirred at 100° C. for 16 h under nitrogen. The mixture was concentrated in vacuo and the residue purified by flash chromatography on silica gel 80% EtOAc/PE) to afford the title compound as a yellow solid (100 mg, 30% yield). MS (ES+) C₂₀H₂₆N₆O₄ requires: 414, found: 415 [M+H]⁺.

### Step 2. Synthesis of (1R,3S)-3-(3-((5-(hydroxymethyl)-3-methylpyrazin-2-yl)amino)-1H-pyrazol-5-yl)cyclopentyl(1-methylcyclopropyl)carbamate (Example 47)

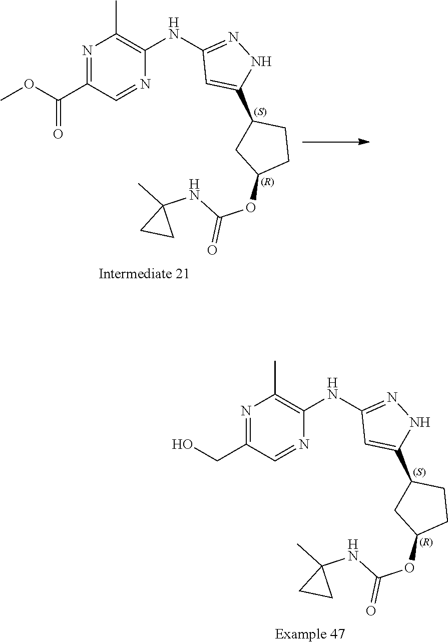

To a solution of Intermediate 21 (90 mg, 217 μmol) in THE (5 mL) was added LiAlH₄ (82 mg, 2.17 mmol) at 0° C. and the mixture stirred for 3 h. The reaction was quenched with water and extracted with ethyl acetate. The combined organics was washed with water, brine, dried (Na₂SO₄) and evaporated to dryness in vacuo. The residue was purified by prep-HPLC (Xtimate, 21.2×250 mm, 10 mm; 5-95% MeCN/H₂O (0.1% NH₄HCO₃) to afford the title product as a white solid (9.7 mg, 11% yield). MS (ES+) C₁₉H₂₆N₆O₃ requires: 386, found: 387 [M+H]⁺. ¹H-NMR (400 MHz, DMSO-d₆) δ ppm 11.96 (br. s, 1H), 8.53 (s, 1H), 7.97 (s, 1H), 7.33 (s, 1H), 6.28 (s, 1H), 5.20 (t, J=6.0 Hz, 1H), 4.98 (s, 1H), 4.44 (d, J=5.6 Hz, 2H), 3.05-3.03 (m, 1H), 2.45 (s, 3H), 2.02-1.62 (m, 6H), 1.24 (s, 3H), 0.61-0.48 (m, 4H).

### Example 48 (1R,3S)-3-(3-((2-(methylamino)pyrimidin-4-yl)amino)-1H-pyrazol-5-yl)cyclopentyl(1-methylcyclopropyl)carbamate

### Step 1. Synthesis of benzyl tert-butyl (4-chloropyrimidin-2-yl)(methyl) carbamate (Intermediate 22)

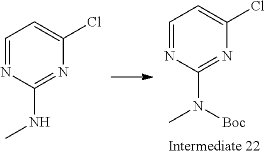

A mixture of 4-chloro-N-methylpyrimidin-2-amine (200 mg, 1.40 mmol), di-tert-butyl dicarbonate (457 mg, 2.09 mmol), DMAP (68 mg, 0.56 mmol) and Et₃N (554 mg, 5.66 mmol) in dry THF (5 mL) was stirred at rt for 1 h. The reaction mixture was evaporated to dryness and the residue was purified by flash chromatography on silica gel eluting with PE/EA(1/1) to afford the title compound (283 mg, 97% yield) as a yellow solid. MS (ES+) C₁₀H₁₄ClN₃O₂ requires: 243, found: 144 [M+H−tBu]⁺.

### Step 2. Synthesis of tert-butyl (4-((1-(tert-butyl)-3-((1S,3R)-3-(((1-methylcyclopropyl)carbamoyl)oxy)cyclopentyl)-1H-pyrazol-5-yl)amino)pyrimidin-2-yl)(methyl)carbamate (Intermediate 23)

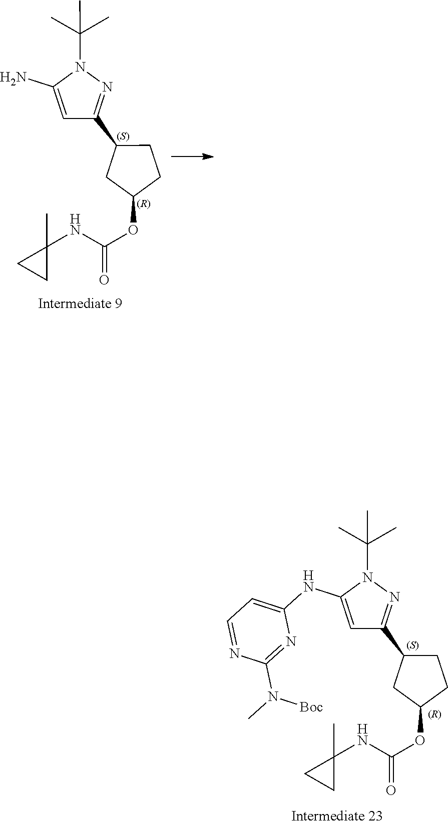

The title compound was prepared as a yellow solid (35 mg, 21% yield) from Intermediate 9 and Intermediate 22 using an analogous method to that described for Example 29, Step 1. MS (ES+) C₂₇H₄₁N₇O₄ requires: 527, found: 528 [M+H]⁺.

### Step 3. Synthesis of (1R,3S)-3-(1-(tert-butyl)-5-((2-(methylamino)pyrimidin-4-yl)amino)-1H-pyrazol-3-yl)cyclopentyl(1-methylcyclopropyl)carbamate (Intermediate 24)

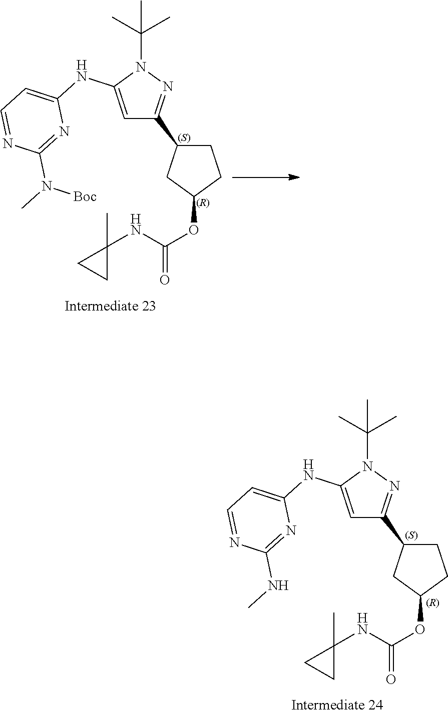

A mixture of Intermediate 23 (35 mg, 4.62 mmol) and HCl (4.0 M in 1,4-dioxane, 2 mL) in DCM (2 mL) was stirred at rt for 0.5 h. The reaction mixture was evaporated under reduced pressure to provide the title compound as a yellow solid (25 mg, 54% yield). MS (ES+) C₂₂H₃₃N₇O₂ requires: 427, found: 428 [M+H]⁺.

### Step 4. Synthesis of (1R,3S)-3-(3-((2-methylamino)pyrimidin-4-yl)amino)-1H pyrazol-5-yl)cyclopentyl(1-methylcyclopropyl)carbamate (Example 53)

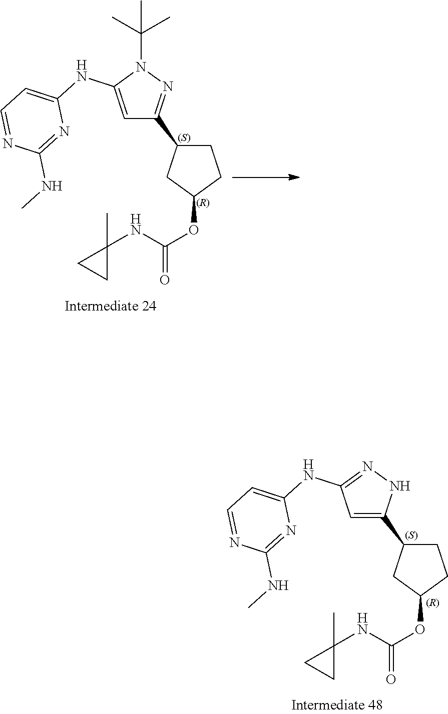

A solution of Intermediate 24 (25 mg, 58.5 μmol) in formic acid (2 mL) was stirred at 80° C. for 3 h. The mixture was concentrated in vacuum and the residue purified using prep-HPLC (Xtimate, 21.2×250 mm, 10 mm; 5-95% MeCN/H₂O (0.1% N₄HCO₃) to afford the title compound as a yellow solid (12.3 mg, 56% yield). MS (ES+) C₁₈H₂₅N₇O₂ requires: 371, found: 372 [M+H]⁺. ¹H-NMR (400 MHz, DMSO-d₆) δ ppm 11.93 (br. s., 1H), 9.34 (br. s., 1H), 7.80 (s, 1H), 7.34 (s, 1H), 6.56 (s, 1H), 6.42 (s, 1H), 6.21 (s, 1H), 4.97 (s, 1H), 3.02-2.98 (m, 1H), 2.78 (d, J=4.8 Hz, 3H), 2.01-1.68 (m, 6H), 1.22 (m, 3H), 0.58-0.47 (m, 2H), 0.48-0.45 (m, 2H).

### Example 49 (1R,3S)-3-(3-((5-(hydroxymethyl)pyrazin-2-yl)amino)-1H-pyrazol-5-yl)cyclopentyl 2,2-dimethylazetidine-1-carboxylate

### Step 1. Synthesis of (1R,3S)-3-(5-amino-1-(tert-butyl)-1H-pyrazol-3-yl)cyclopentan-1-ol (Intermediate 25)

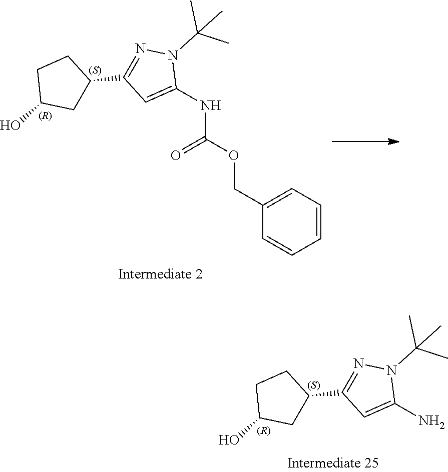

A mixture of Intermediate 2 (1.69 g, 3.71 mmol) and Pd/C (10%, 800 mg) in EtOAc (20 mL) was degassed and purged with hydrogen (3 cycles) and stirred at rt for 16 h under a hydrogen balloon. The reaction mixture was filtered through celite, and the filtrate evaporated under reduced pressure to afford the title compound as a red solid (960 mg, 81% yield). MS (ES+) C₁₂H₂₁N₃O requires: 223, found: 224 [M+H]⁺.

### Step 2. Synthesis of methyl 5-((1-(tert-butyl)-3-((1S,3R)₃-hydroxycyclopentyl)-1H-pyrazol-5-yl)amino)pyrazine-2-carboxylate (Intermediate 26)

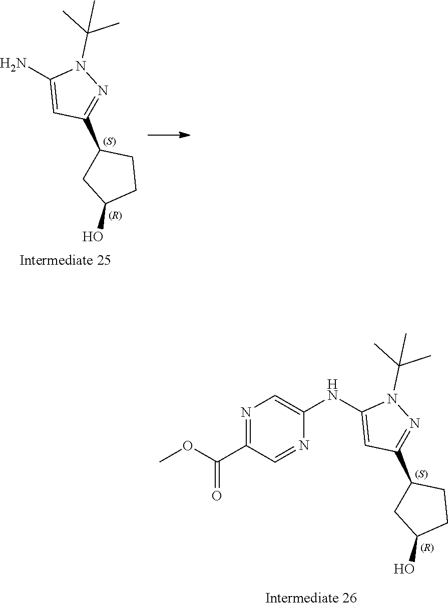

A mixture of Intermediate 25 (200 mg, 895 μmol), methyl 5-chloropyrazine-2-carboxylate (307 mg, 1.78 mmol), BrettPhos Pd G4 (137 mg, 89.5 μmol) and KOAc (263 mg, 2.68 mmol) in dioxane (10 mL) was stirred at 90° C. for 16 h under nitrogen. The mixture was evaporated to dryness in vacuo and the residue was purified by flash chromatography on silica gel (9:1 EtOAc/PE) to afford the title compound as a yellow oil (200 mg, 62% yield). MS (ES+) C₁₈H₂₅N₅O₃ requires: 359, found: 360 [M+H]⁺.

### Step 3 Synthesis of methyl 5-((1-(tert-butyl)-3-(1S,3R)-3-(((4-nitrophenoxy)carbonyl)oxy)cyclopentyl)-1H-pyrazol-5-yl)amino)pyrazine-2-carboxylate (Intermediate 27)

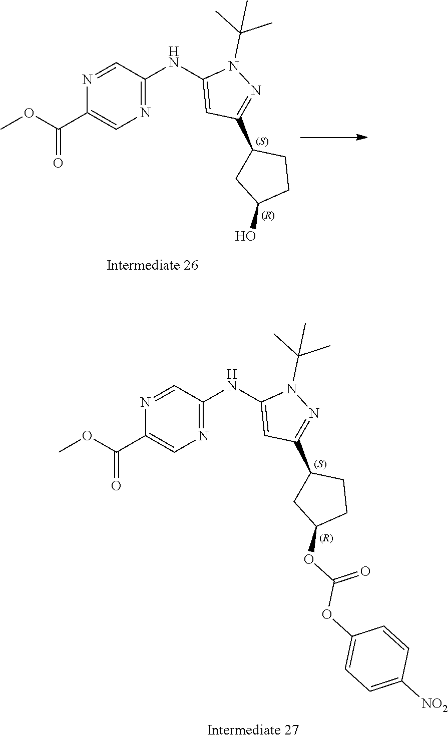

To a mixture of Intermediate 26 (200 mg, 556 μmol), DMAP (6.79 mg, 55.6 μmol) and pyridine (219 mg, 2.78 mmol) in dichloromethane (5 mL) and dry THF (5 mL) was added 4-nitrophenyl carbonochloridate (1.12 g, 5.56 mmol) and the resulting yellow suspension was stirred at rt for 2 h. The reaction mixture was concentrated to dryness and purified by flash chromatography on silica gel (50% PE/EtOAc) to afford the title compound as a yellow solid (110 mg, 37% yield). MS (ES+) C₂₁H₂₈N₆O₇ requires: 524, found: 525 [M+H]⁺.

### Step 4. Synthesis of methyl 5-((5-((1S,3R)-3-(((4-nitrophenoxy)carbonyl)oxy)cyclopentyl)-1H-pyrazol-3-yl)amino)pyrazine-2-carboxylate (Intermediate 28)

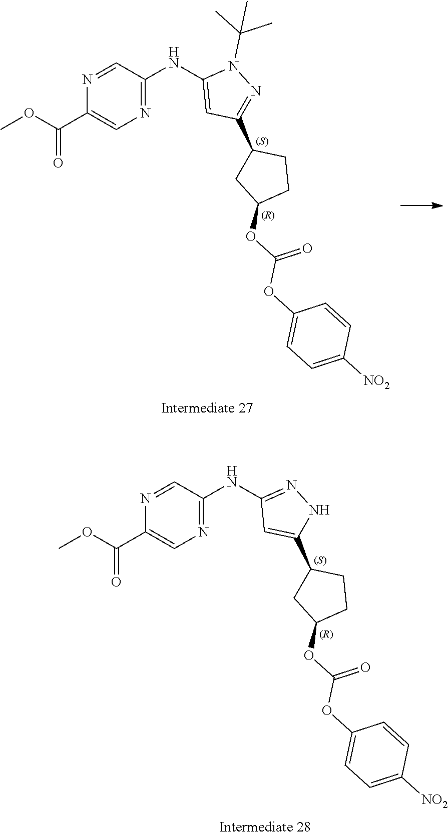

A solution of Intermediate 27 (28 mg, 53.3 μmol) in formic acid (2 mL) was stirred at 80° C. for 2 h. The mixture was evaporated to dryness in vacuo to give the title compound which was used in next step without purification. MS (ES+) C₂₁H₂₀N₆O₇ requires: 468, found: 469 [M+H]⁺.

### Step 5. Synthesis of methyl 5-((5-((1S,3R)-3-((2,2-dimethylazetidine-1-carbonyl)oxy)cyclopentyl)-1H-pyrazol-3-yl)amino)pyrazine-2-carboxylate (Intermediate 29)

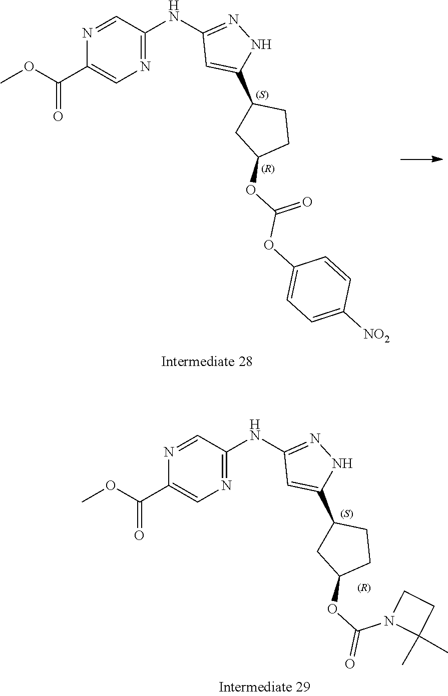

A mixture of Intermediate 28 (110 mg, 234 μmol) and 2,2-dimethylazetidine (59.7 mg, 702 μmol) in dry THF (4 mL) was stirred at rt for 2 h. The reaction mixture was diluted with EtOAc and washed with water and brine. The organic layer was concentrated to give a crude product which was used in the next step without further purification. MS (ES+) C₂₀H₂₆N₆O₄ requires: 414, found: 415 [M+H]⁺.

### Step 6. Synthesis of (1R,3S)-3-(3-((5-(hydroxyethyl)pyrazin-2-yl)amino)-1H-pyrazol-5-yl)cyclopentyl 2,2-dimethylazetidine-1-carboxylate (Example 49)

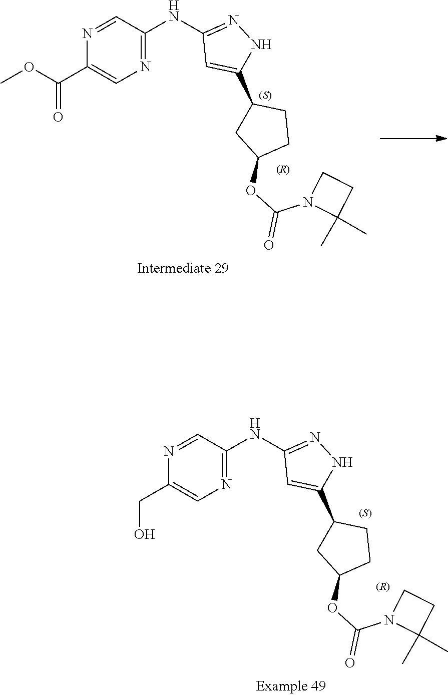

To a solution of Intermediate 29 (20 mg, 48.2 μmol) in dry THF (6 mL) was added LiAlH₄ (3.65 mg, 96.4 μmol) and the mixture stirred at 0° C. for 1 h. The reaction mixture was diluted with EtOAc and washed with water and brine. The organic layer was concentrated to dryness and purified with prep-HPLC (Xtimate, 21.2×250 mm, 10 mm, 5-95% MeCN/H₂O (0.1% NH₄HCO₃) to afford the title compound as a white solid (5.1 mg, 27% yield). MS (ES+) C₁₉H₂₆N₆O₃ requires: 386, found: 387 [M+H]⁺. ¹H-NMR (400 MHz, DMSO-d₆) δ ppm 11.95 (br. s., 1H), 9.53 (s, 1H), 8.48 (s, 1H), 8.10 (s, 1H), 6.18 (s, 1H), 5.24 (t, J=5.6 Hz, 1H), 5.06-4.94 (m, 1H), 4.45 (d, J=5.6 Hz, 2H), 3.75 (t, J=7.6 Hz, 1H), 3.65 (t, J=7.6 Hz, 1H), 3.12-3.04 (m, 1H), 2.46-2.32 (m, 1H), 2.08-1.99 (m, 1H), 1.99-1.91 (m, 2H), 1.91-1.81 (m, 1H), 1.81-1.58 (m, 3H), 1.37 (s, 1H), 1.34 (s, 3H).

### Example 50 (1R,3S)-3-(3-((5-(2-hydroxypropan-2-yl)pyrazin-2-yl)amino)-1H-pyrazol-5-yl)cyclopentyl 2,2-dimethylazetidine-1-carboxylate

### Step 1. Synthesis of 1-(5-((1-(tert-butyl)-3-((1S,3R)-3-hydroxycyclopentyl)-1H-pyrazol-5-yl)amino)pyrazin-2-yl)ethan-1-one (Intermediate 30)

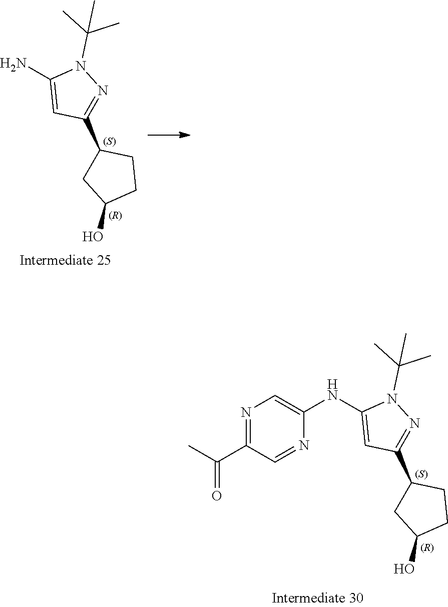

The title compound was prepared from Intermediate 25 using an analogous method to that described for Example 54, Step 2. Red solid (900 mg, 97%); MS (ES+) C₁₈H₂₅N₅O₂ requires: 343, found: 344 [M+H]⁺.

### Step 2. Synthesis of (1R,3S)-3-(5-((5-acetylpyrazin-2-yl)amino)-1-(tert-butyl)-1H-pyrazol-3-yl)cyclopentyl(4-nitrophenyl) carbonate (Intermediate 31)

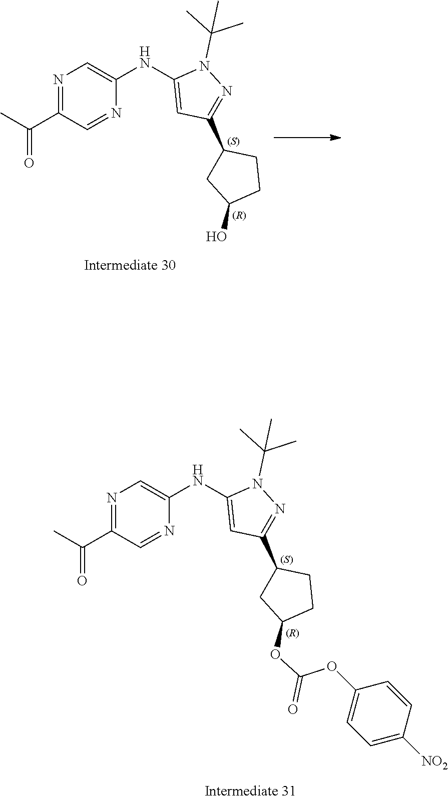

The title compound was prepared from Intermediate 30 using an analogous method to that described for Example 54, Step 3. Yellow solid (730 mg, 56% yield). MS (ES+) C₂₅H₂₈N₆O₆ requires: 508, found: 509 [M+H]⁺.

### Step 3. Synthesis of (1R,3S)-3-(3-((5-acetylpyrazin-2-yl)amino)-1H-pyrazol-5-yl)cyclopentyl(4-nitrophenyl) carbonate (Intermediate 32)

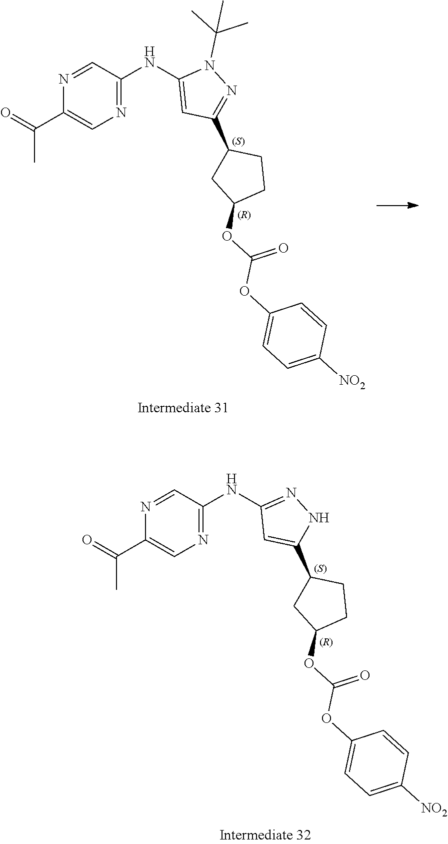

The title compound was prepared from Intermediate 31 using an analogous method to that described for Example 54, Step 4. Yellow solid (580 mg, 93% yield). MS (ES+) C₂₁H₂₀N₆O₆ requires: 452, found: 453 [M+H]⁺.

### Step 4. Synthesis of (1R,3S)-3-(3-((5-acetylpyrazin-2-yl)amino)-1H-pyrazol-5-yl)cyclopentyl 2,2-dimethylazetidine-1-carboxylate (Intermediate 33)

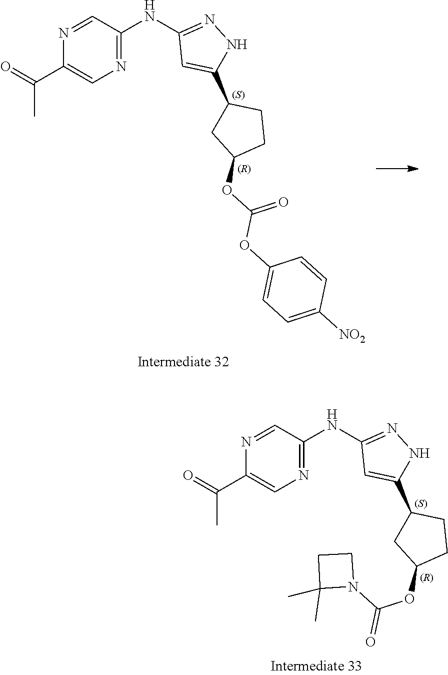

The title compound was prepared from Intermediate 32 using an analogous method to that described for Example 54, Step 5. Yellow solid (430 mg, 84% yield). MS (ES+) C₂₀H₂₆N₆O₃ requires: 398, found: 399 [M+H]⁺.

### Step 5. Synthesis of (1R,3S)-3-(3-((5-(2-hydroxypropan-2-yl)pyrazin-2-yl)amino)-1H-pyrazol-5-yl)cyclopentyl 2,2-dimethylazetidine-1-carboxylate (Example 50)

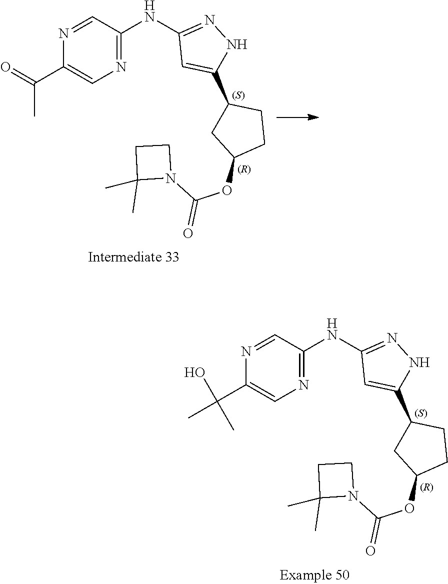

To a solution of Intermediate 33 (130 mg, 326 μmol) in dry THF (5 mL) was added CH₃Li (3.0 M, 0.7 ml, 0.21 mmol) and the mixture stirred at 0° C. for 1 h. The reaction mixture was diluted with EtOAc and washed with water and brine. The organic layer was concentrated and the residue purified with prep-HPLC (Xtimate, 21.2×250 mm, 10 mm; 5-95% MeCN/H₂O (0.1% NH₄HCO₃) to afford the title compound as a white solid (18.2 mg, 13% yield). MS (ES+) C₂₁H₃₀N₆O₃ requires: 414, found: 415 [M+H]⁺. ¹H-NMR (500 MHz, DMSO-d₆) δ ppm 11.91 (br, s., 1H), 9.43 (s, 1H), 8.44 (s, 1H), 8.30 (s, 1H), 6.16 (s, 1H), 5.13 (s, 1H), 5.03-4.94 (m, 1H), 3.77-3.74 (m, 1H), 3.67-3.64 (m, 1H), 3.11-3.04 (m, 1H), 2.47-2.43 (m, 1H), 2.06-1.99 (m 1H), 1.96-1.89 (m, 2H), 1.89-1.82 (m, 1H), 1.82-1.62 (m, 31H), 1.41 (s, 6H), 1.37 (s, 3H), 1.34 (s, 3H).

### Example 51 and 52 (1R,3S)-3-(3-((5-((R)-1-hydroxyethyl)pyrazin-2-yl)amino)-1H-pyrazol-5-yl)cyclopentyl 2,2-dimethylazetidine-1-carboxylate and (1R,3S)-3-(3-((5-((S)-1-hydroxyethyl)pyrazin-2-yl)amino)-1H-pyrazol-5-yl)cyclopentyl 2,2-dimethylazetidine-1-carboxylate

### Step 1. Synthesis of (1R,3S)-3-(3-((5-((R)-1-hydroxyethyl)pyrazin-2-yl)amino)-1H-pyrazol-5-yl)cyclopentyl 2,2-dimethylazetidine-1-carboxylate (Intermediate 34)

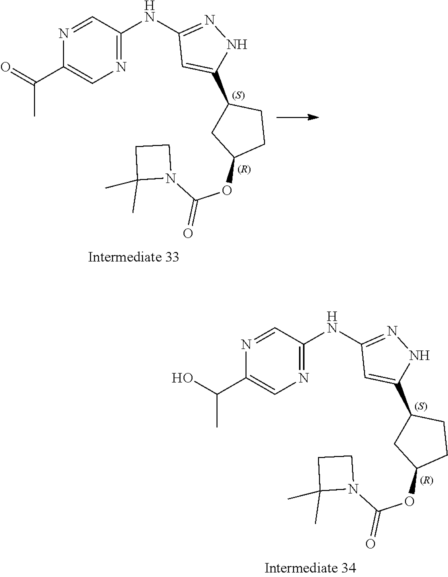

NaBH₄ (37.8 mg, 1.00 mmol) was added to a solution of Intermediate 33 (200 mg, 501 μmol) in MeOH (5 mL) and the mixture stirred at 0° C. for 1 h. The reaction mixture was diluted with EtOAc and washed with water and brine. The organic layer was concentrated to dryness and the residue purified by flash chromatography on silica gel (6:1 DCM/MeOH) to afford the title compound as a white solid (200 mg, 100% yield). MS (ES+) C₂₀H₂₈N₆O₃ requires: 400, found: 401 [M+H]⁺.

### Step 2. Synthesis of (1R,3S)-3-(3-((5-((R)-1-hydroxyethyl)pyrazin-2-yl)amino)-1H-pyrazol-5-yl)cyclopentyl 2,2-dimethylazetidine-1-carboxylate and (1R,3S)-3-(3-((5-((S)-1-hydroxyethyl)pyrazin-2-yl)amino)-1H-pyrazol-5-yl)cyclopentyl 2,2-dimethylazetidine-1-carboxylate (Example 56 and 57)

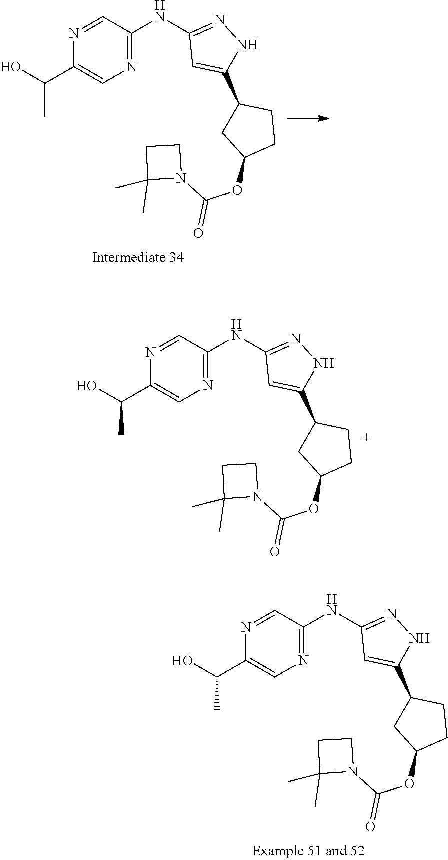 [SMILES: CC(O)c1cnc(Nc2cc([C@H]3CC[C@@H](OC(=O)N4CCC4(C)C)C3)[nH]n2)cn1 ; C[C@@H](O)c1cnc(Nc2cc([C@H]3CC[C@@H](OC(=O)N4CCC4(C)C)C3)[nH]n2)cn1 ; C[C@H](O)c1cnc(Nc2cc([C@H]3CC[C@@H](OC(=O)N4CCC4(C)C)C3)[nH]n2)cn1]

Intermediate 34 (200 mg) was separated by chiral-SFC (Diacel AD, 20×250 mm, 10 mm; 50% IPA (0.5% Methanolic ammonia) in CO₂) to afford:

Peak 1, Example 51 (65.5 mg, 33%); (1R,3S)-3-(3-((5-((R)-1-hydroxyethyl)pyrazin-2-yl)amino)-1-H-pyrazol-5-yl)cyclopentyl 2,2-dimethylazetidine-1-carboxylate or (1R,3S)-3-(3-((5-((S)-1-hydroxyethyl)pyrazin-2-yl)amino)-1H-pyrazol-5-yl)cyclopentyl 2,2-dimethylazetidine-1-carboxylate. MS (ES+) C₂₀H₂₈N₆O₃ requires: 400, found: 401 [M+H]⁺. ¹H-NMR (400 MHz, DMSO-d₆): 11.96 (br. s., 1H), 9.48 (s, 1H), 8.48 (s, 1H), 8.14 (s, 1H), 6.18 (s, 1H), 5.23 (d, J=4.8 Hz, 1H), 5.03-4.96 (m, 1H), 4.69-4.67 (m, 1H), 3.76 (t, J=8.0 Hz, 1H), 365 (t, J=76 Hz, 1H), 3.12-3.04 (m, 1H) 2.46-2.33 (m, 1H), 2.09-1.99 (m, 1H), 1.99-1.91 (m, 2H), 1.91-1.81 (m, 1H), 1.81-1.59 (m, 3H), 1.37-1.34 (m, 9H).

Peak 2, Example 52 (50.8 mg, 25%); (1R,3S)-3-(3-((5-((R)-1-hydroxyethyl)pyrazin-2-yl)amino)-1H-pyrazol-5-yl)cyclopentyl 2,2-dimethylazetidine-1-carboxylate or (1R,3S)-3-(3-((5-((S)-1-hydroxyethyl)pyrazin-2-yl)amino)-1H-pyrazol-5-yl)cyclopentyl 2,2-dimethylazetidine-1-carboxylate. NIS (ES+) C₂₀H₂₈N₆O₃ requires: 400, found: 401 [M+H]+. ¹H-NMR (400 MHz, DMSO-d₆): 11.96 (br. s., 1H), 9.48 (s, 1H), 8.48 (s, 1H), 8.14 (s, 1H), 6.18 (s, 1H), 5.24 (d, J=4.8 Hz, 1H), 5.03-4.94 (m, 1H), 4.71-4.65 (m, 1H), 3.76 (t, J=8.0 Hz, 1H), 3.65 (t, J=7.6 Hz, 1H), 3.12-3.04 (m, 1H), 2.48-2.36 (m, 1H), 2.07-1.99 (m, 1H), 1.99-1.91 (m, 2H), 1.91-1.77 (m, 1H), 1.77-1.59 (m, 3H), 1.37-1.34 (m, 9H).

### Example 53 (1R,3S)-3-(3-((5-cyanopyrazin-2-yl)amino)-1H-pyrazol-5-yl)cyclopentyl isopropylcarbamate

### Step 1. Synthesis of benzyl (1-(tert-butyl)-3-((1S,3R)-3-((isopropylcarbamoyl)oxy)cyclopentyl)-1H-pyrazol-5-yl)carbamate (Intermediate 35)

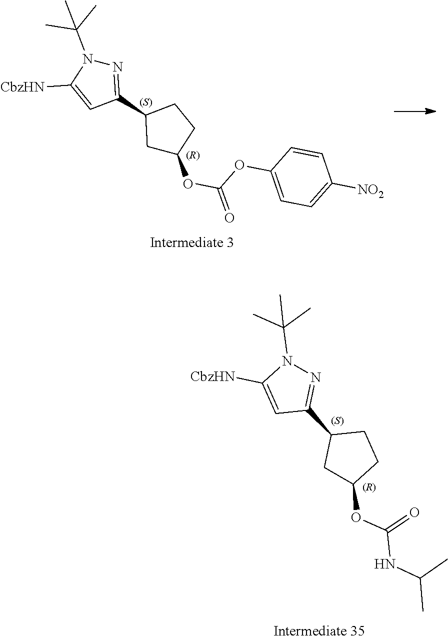

A mixture of Intermediate 3 (2.5 g, 4.78 mmol) and propan-2-amine (845 mg, 14.3 mmol) in THF (40 mL) was stirred at RT for 16 h. The reaction mixture was diluted with EtOAc and washed with water and brine. The organic layer was evaporated to dryness and the residue purified by flash chromatography on silica gel (2:1 PE/EtOAc) to afford the title compound as a yellow solid (2.1 g, 99% yield). MS (ES+) C₂₄H₃₄N₄O₄ requires: 442, found: 443 [M+H]⁺.

### Step 2. Synthesis of (1R,3S)-3-(5-amino-1-(tert-butyl)-1H-pyrazol-3-yl)cyclopentyl isopropylcarbamate (Intermediate 36)

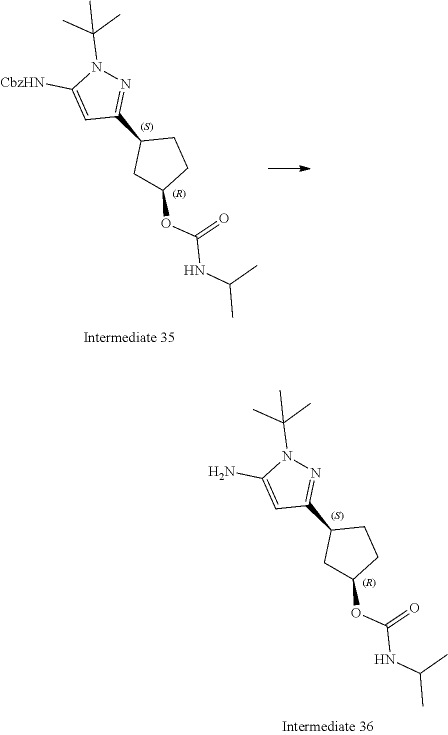

A mixture of Intermediate 35 (2.1 g, 4.74 mmol) and Pd/C (10%, 1.1 g) in EtOAc (40 mL) was degassed and purged with hydrogen (3 cycles) and then stirred at rt for 16 h under a hydrogen balloon. The reaction mixture was filtered through celite and the filtrate was evaporated under reduced pressure to afford the title compound as a black oil (1.4 g, 95% yield). MS (ES+) C₁₆H₂₈N₄O₂ requires: 308, found: 309 [M+H]⁺.

### Step 3. Synthesis of (1R,3S)-3-(1-(tert-butyl)-5-((5-cyanopyrazin-2-yl)amino)-1H-pyrazol-3-yl)cyclopentyl isopropylcarbamate (Intermediate 37)

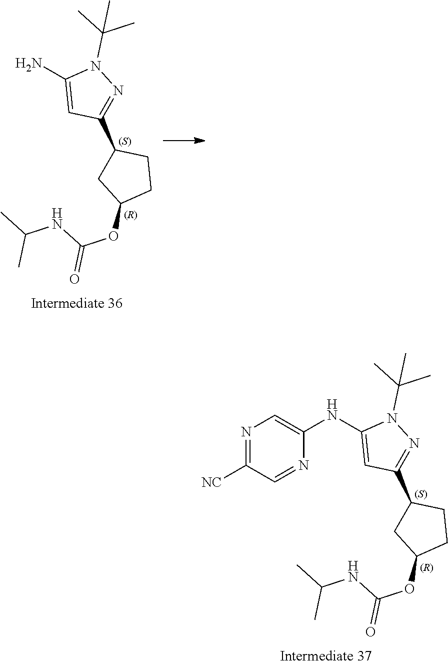

To a solution of Intermediate 36 (236 mg, 766 mol) in anhydrous THF (5 mL) was added LiHMDS (3.0 M in THF, 3 mL, 9.0 mmol) at 0° C. After stirring at 0° C. for 10 minutes a solution of 5-chloropyrazine-2-carbonitrile (107 mg, 766 μmol) in THE (1.0 mL) was added and the reaction mixture stirred at rt for 1 h. The reaction mixture was diluted with EtOAc and washed with water and brine. The organic layer was evaporated to dryness and the residue purified by flash chromatography on silica gel (1:2 EtOAc/PE) to afford the title compound as a yellow oil (90 mg, 28% yield). MS (ES+) C₂₁H₂₉N₇O₂ requires: 411, found: 412 [M+H]⁺.

### Step 4. Synthesis of (1R,3S)-3-(3-((5-cyanopyrazin-2-yl)amino)-1H-pyrazol-5-yl)cyclopentyl isopropylcarbamate (Example 53)

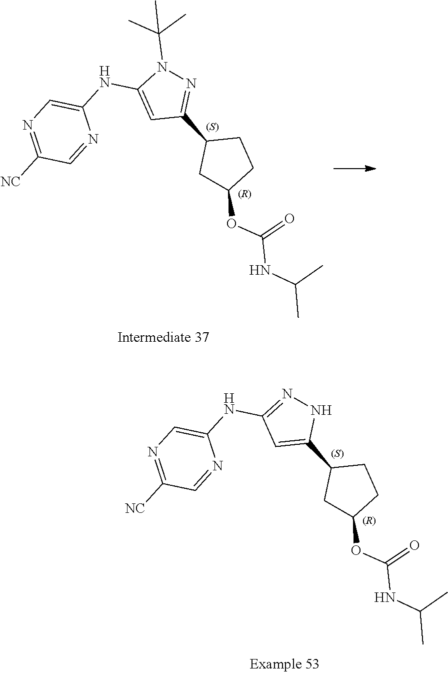

A solution of Intermediate 37 (70 mg, 170 μmol) in HCO₂H was stirred at 80° C. for 14 h. The reaction mixture was evaporated to dryness in vacuo and the residue purified by prep-HPLC (Xtimate, 21.2×250 mm, 10 mm; 5-95% MeCN/H₂O (0.1% NH₄HCO₃) to afford the title compound as a brown solid (8.8 mg, 14% yield). MS (ES+) C₁₇H₂₁N₇O₂ requires: 355, found: 356 [M+H]⁺. ¹H-NMR (500 MHz, DMSO-d₆) δ ppm 12.37 (br. s., 1H), 10.64 (s, 1H), 8.62 (s, 1H), 8.46 (s, 1H), 6.95 (d, J=7.5 Hz, 1H), 6.33 (s, 1H), 4.99 (s, 1H), 3.57-3.56 (m, 1H), 3.09-3.06 (m, 1H), 2.03-2.02 (m, 1H), 1.92-1.88 (m, 1H), 1.73-1.59 (m, 3H), 1.03-1.02 (m, 6H).

### Example 54 (1R,3S)-3-(3-((3-methoxy-1,2,4-triazin-5-yl)amino)-1H-pyrazol-5-yl)cyclopentyl(1-methylcyclopropyl)carbamate

### Step 1. Synthesis of (1R,3S)-3-(1-(tert-butyl)-5-((3-methoxy-1,2,4-triazin-5-yl)amino)-1H-pyrazol-3-yl)cyclopentan-1-ol (Intermediate 38)

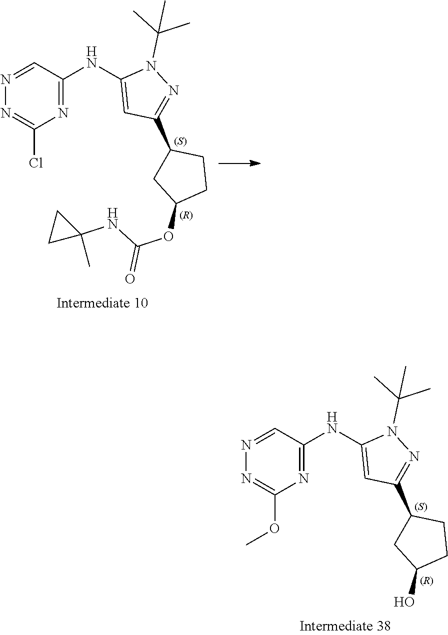

A mixture Intermediate 10 (110 mg, 253 μmol) and MeONa (273 mg, 5.06 mmol) in NMP (2.5 mL) was stirred at 160° C. for 5 h. The reaction mixture was diluted with EtOAc and washed with water and brine. The combined organics were evaporated to dryness in vacuo and the residue was purified by prep-HPLC (Xtimate, 21.2×250 mm, 10 mm; 5-95% MeCN/H₂O (0.1% NH₄HCO₃) to afford the title compound as a white solid (33 mg, 39% yield). MS (ES+) C₁₆H₂₄N₆O₂ requires: 332, found: 333 [M+H]⁺.

### Step 2. Synthesis of (1R,3S)-3-(1-(tert-butyl)-5-((3-methoxy-1,2,4-triazin-5-yl)amino)-1H-pyrazol-3-yl)cyclopentyl(4-nitrophenyl) carbonate (Intermediate 39)

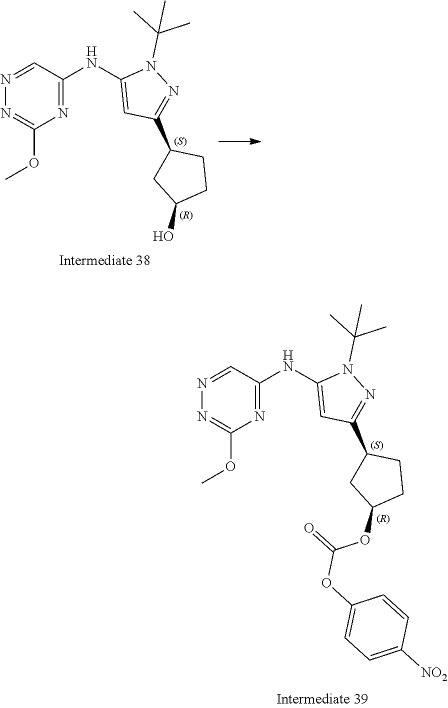

A mixture of Intermediate 38 (33 mg, 99.2 mol) in DCM (2.5 mL) and dry THF (2.5 mL) was treated with DMAP (1.21 mg, 9.92 μmol), pyridine (39.2 mg, 496 μmol) and 4-nitrophenyl carbonochloridate (199 mg, 992 μmol) at 0′° C. The resulting yellow suspension was stirred at rt for 2 h. The reaction mixture was evaporated to dryness in vacuo and the residue purified by flash chromatography on silica gel (2:3 PE/EtOAc) to afford the title compound as a yellow solid (40 mg, 81% yield). MS (ES+) C₂₃H₂₇N₇O₆ requires: 497, found: 498 [M+H]⁺.

### Step 3. Synthesis of (1R,3S)-3-(1-(tert-butyl)-5-((3-methoxy-1,2,4-triazin-5-yl)amino)-1H-pyrazol-3-yl)cyclopentyl(1-methylcyclopropyl)carbamate. (Intermediate 40)

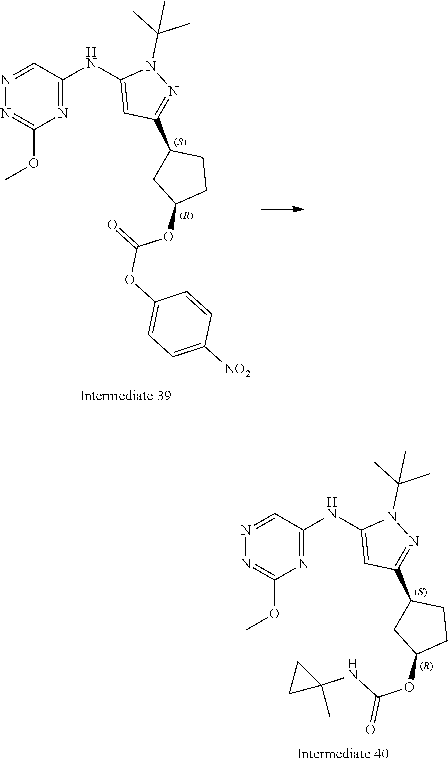

A mixture of Intermediate 39 (40 mg, 80.4 μmol), DIEA (51.9 mg, 402 μmol) and 1-methylcyclopropan-1-amine hydrochlroide (25.9 mg, 241 μmol) in DMF (1 mL) was stirred at 60° C. for 16 h. The reaction mixture was diluted with EtOAc and washed with water ad brie. The organic layer was concenrated to dryness the residue purified by flash chromatography on silica gel (50% PE/EtOAc) to afford the title compound as a yellow solid (20 mg, 57% yield). MS (ES+) C₂₁H₃₁N₇O₃ requires: 429, found: 430 [M+H]⁺.

### Step 4. Synthesis of (1R,3S)-3-(3-((3-methoxy-1,2,4-triazin-5-yl)amino)-1H-pyrazol-5-yl)cyclopentyl(1-methylcyclopropyl)carbamate (Example 54)

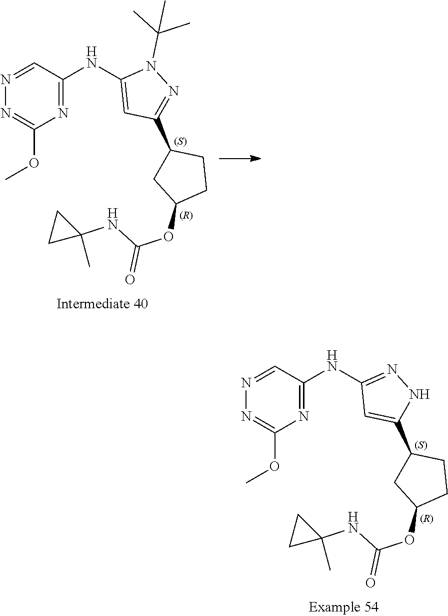

A solution of Intermediate 40 (20 mg, 41.9 μmol) in formic acid (1 mL) was stirred at 80° C. for 3 h. The mixture was evaporated to dryness in vacuo and the residue purified by prep-HPLC (Xtimate, 21.2×250 mm, 10 mm; 5-95% MeCN/H₂O (0.1% NH₄HCO₃) to afford the title compound as a white solid (1.5 mg, 8% yield). MIS (ES+) C₁₇H₂₃N₇O₃ requires: 372, found: 373 [M+H]⁺. ¹H-NMR (400 MHz, DMSO-d₆) δ ppm 12.32 (br. s., 1H), 10.73 (s, 1H), 8.48 (s, 1H), 7.34 (s, 1H), 6.54 (s, 1H), 4.99 (s, 1H), 3.94 (s, 31), 3.09-3.05 (m, 1H), 2.49-2.45 (m, 1H), 2.03-1.58 (m, 5H), 1.23 (s, 3H), 0.57-0.46 (m, 4H).

### Example 55 (1R,3S)-3-(3-((5-hydroxymethyl)pyrazin-2-yl)amino)-1H-pyrazol-5-yl)cyclopentyl(1-methylcyclopropyl)carbaminate

### Step 1. Synthesis of methyl 5-((1-(tert-butyl)-3-((1S,3R)-3-(((1-methylcyclopropyl)carbamoyl)oxy)cyclopentyl)-1H-pyrazol-5-yl)amino)pyrazine-2-carboxylate (Intermediate 41)

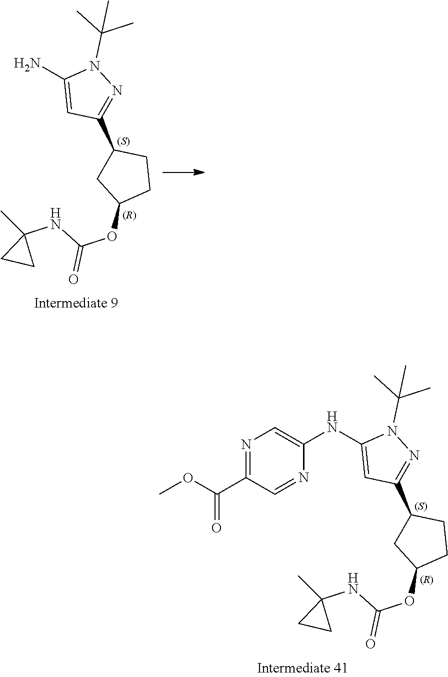

A mixture of Intermediate 9 (50 mg, 156 mol), methyl 5-chloropyrazine-2-carboxylate (40.3 mg, 234 μmol), BrettPhos Pd G₄ (23.9 mg, 15.6 μmol) and KOAc (76.5 mg, 780 μmol) in dioxane (2 mL) was stirred at 90° C. for 16 h under nitrogen. The mixture was evaporated to dryness in vacuo and the residue purified by flash chromatography on silica gel (50% EtOAc/PE) to afford the title compound as a yellow oil (29.7 mg, 41% yield). MS (ES+) C₂₃H₃₂N₆O₄ requires: 456, found: 457 [M+H]⁺.

### Step 2. Synthesis of methyl 5-(5-((1S,3R)-3-(((1-methylcyclopropyl)carbamoyl)oxy)cyclopentyl)-1H-pyrazol-3-yl)amino)pyrazine-2-carboxylate (Intermediate 42)

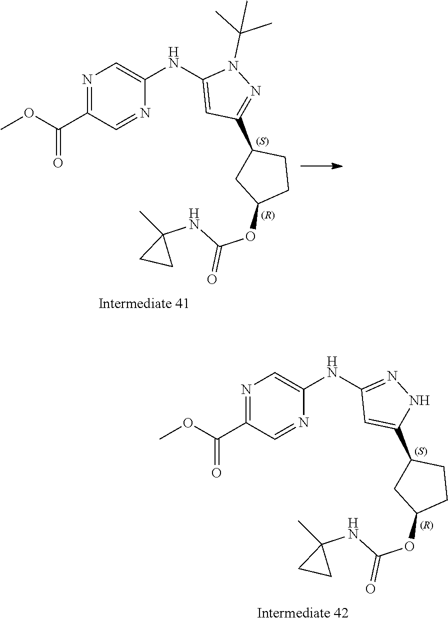

A solution of Intermediate 41 (29.7 mg, 65.0 μmol) in formic acid (1 mL) was stirred at 80° C. for 3 h. The reaction mixture was evaporated to dryness and the residue was used to the next step directly. MS (ES+) C₁₉H₂₄N₆O₄ requires: 400, found: 401 [M+H]⁺.

### Step 3. Synthesis of (1R,3S)-3-(3-((5-(hydroxymethyl)pyrazin-2-yl)amino)-1H-pyrazol-5-yl)cyclopentyl(1-methylcyclopropyl)carbamate (Example 55)

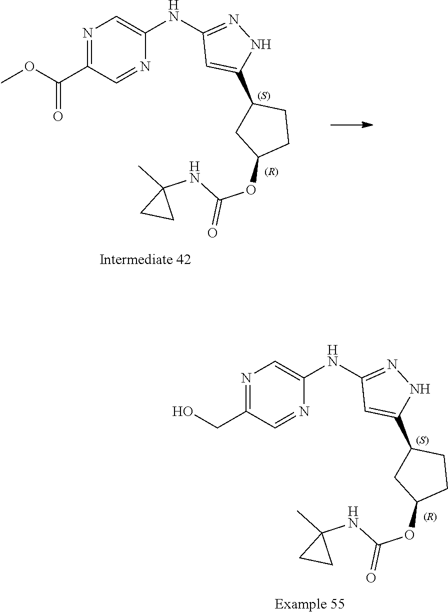

LiAlH₄ (19 mg 0.5 mmol) was added to a solution of Intermediate 42 (20 mg, 49.9 μmol) in dry THF (6 mL) and the mixture stirred at rt for 1 h. The reaction mixture was diluted with EtOAc and washed with, ater and brine. The organic layer was evaporated to dryness and the residue purified by prep-HPLC (Xtimate, 21.2×250 mm, 10 mm; 5-95% MeCN/H₂O (0.1% NH₄HCO₃) to afford the title compound as a white solid (3.2 mg, 17% yield). MS (ES+) C₁₈H₂₄N₆O₃ requires: 372, found: 373 [M+H]⁺. ¹H-NMR (400 MHz, DMSO-d₆) δ ppm 11.94 (br. s., 1H), 9.51 (s, 1H), 8.50 (s, 1H), 8.11 (s, 1H), 7.36 (s, 1H), 6.14 (s, 1H), 5.25 (t, J=5.6 Hz, 1H), 4.98 (br. s., 1H), 4.45 (d, J=5.6 Hz, 2H), 3.13-2.96 (m, 1H), 2.46-2.41 (m, 1H), 208-1.95 (m, 1H), 1.95-1.77 (m, 1H), 1.77-1.62 (m, 2H), 1.62-1.51 (m, 1H), 1.23 (s, 3H), 0.59-0.47 (m, 4H).

### Example 56 (1R,3S)-3-(3-((6-(cyclopropylmethoxy)pyrazin-2-yl)amino)-1H-pyrazol-5-yl)cyclopentyl methylcarbamate

### Step 1. Synthesis of benzyl (1-(tert-butyl)-3-((1S,3R)-3-((methylcarbamoyl)oxy)cyclopentyl)-1H-pyrazol-5-yl)carbamate (Intermediate 43)

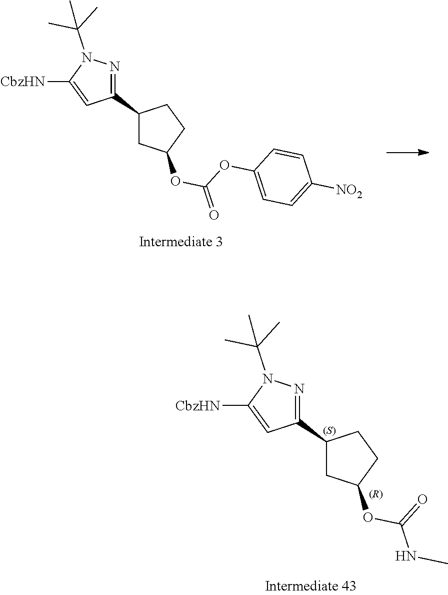

DIPEA (74 mg, 0.57 mmol) was added to Intermediate 3 (100 mg, 0.19 mmol) and methylamine (59 mg, 1.9 mmol) in DMF (2 mL) and the mixture stirred at 60° C. for 16 h. The reaction mixture was diluted with EtOAc and washed with water and brine. The combined organics were evaporated to dryness in vacuo and the residue purified by flash chromatography on silica gel (50% EtOAc/PE) to afford the title compound as a yellow solid (40 mg, 51%). MS (ES+) C₂₂H₃₀N₄O₄ requires: 414, found: 415 [M+H]⁺.

### Step 2. Synthesis of (1R,3S)-3-(5-amino-1-(tert-butyl)-1H-pyrazol-3-yl)cyclopentyl methylcarbamate (Intermediate 44)

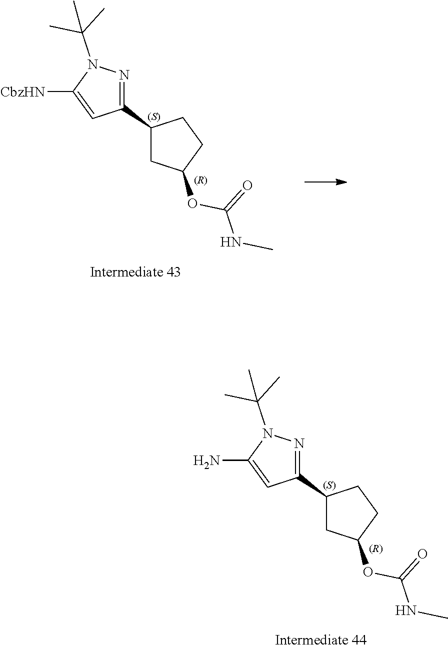

A mixture of Intermediate 43 (40 mg, 97 μmol) and Pd/C (10%, 205 mg) in EtOAc (5 mL) was degassed and purged with hydrogen (3 cycles) and then stirred at rt for 16 h under a hydrogen balloon. The reaction mixture was filtered through celite and the filtrate evaporated under reduced pressure to afford the title compound as a pink solid (40 mg, 100%) which was used without further purification. MS (ES+) C₁₄H₂₄N₄O₂ requires: 280, found: 281 [M+H]⁺.

### Step 3. Synthesis of 2-chloro-6-(cyclopropylmethoxy)pyrazine (Intermediate 45)

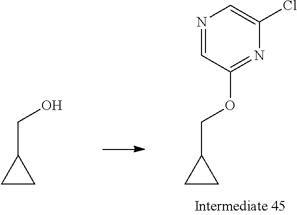 [SMILES: OCC1CC1 ; Clc1cncc(OCC2CC2)n1]

To an ice-cold solution of cyclopropylmethanol (200 mg, 2.77 mmol) in THF (8 mL) was added sodium hydride (66.4 mg, 2.77 mmol) and the mixture stirred for 20 min at rt 2,6-dichloropyrazine (412 mg, 2.77 mmol) was added and the reaction mixture was stirred for another 1 h and quenched with NH₄Cl. aq and extracted with EtOAc. The combined organic layer were evaporated to dryness in vacuo and the residue purified by flash chromatography on silica gel (9:1 PE/EtOAc) to afford the title compound as a yellow solid (500 mg 97.8%). MS (ES+) C₈H₉ClN₂O requires: 184, found: 185 [M+H]⁺.

### Step 4. Synthesis of (1R,3S)-3-(3-((6-(cyclopropylmethoxy)pyrazin-2-yl)amino)-1H-pyrazol-5-yl)cyclopentyl methylcarbamate (Example 56)

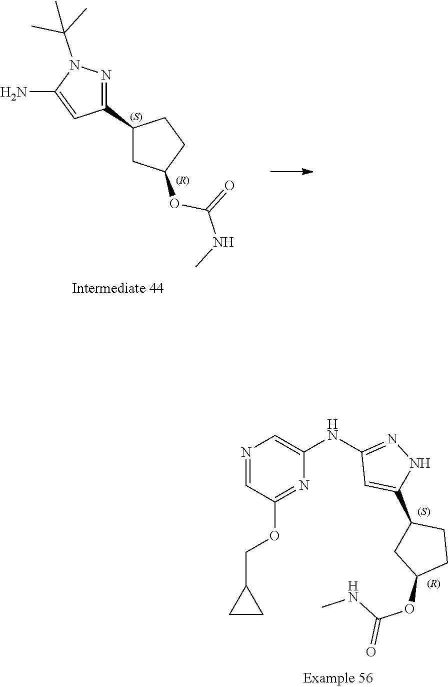

The title compound was prepared as a white solid (4.5 mg, 9% over 2-parts) from (1R,3S)-3-(5-amino-1-(tert-butyl)-1H-pyrazol-3-yl)cyclopentyl methylcarbamate (Intermediate 44) and 2-chloro-6-(cyclopropylmethoxy)pyrazine (Intermediate 45) using an analogous 2-part method as described for Example 24. MS (ES+) C₁₈H₂₄N₆O₃ requires: 372, found: 373 [M+H]+. ¹H-NMR (400 MHz, DMSO-d₆) δ ppm 11.97 (s, 1H), 9.54 (s, 1H), 8.02 (s, 1H), 7.50 (s, 1H), 6.89 (s, 1H), 6.23 (s, 1H), 5.00 (s, 1H), 4.12-4.09 (m, 1H), 3.09-303 (m, 1H), 2.55-2.53 (m, 3H), 2.03-1.58 (m, 6H), 1.30-1.23 (m, 1H), 0.57-0.53 (m, 2H) 0.34-0.30 (m, 2H).

### Example 57 (1 S,3R)-3-(3-(pyrazin-2-ylamino)-1H-pyrazol-5-vi)cyclopentyl(1-methylcyclopropyl)carbamate

### Step 1. Synthesis of (1S,3R)-3-(5-amino-1-(tert-butyl)-1H-pyrazol-3-yl)cyclopentyl(1-methylcyclopropyl)carbamate (Intermediate 45)

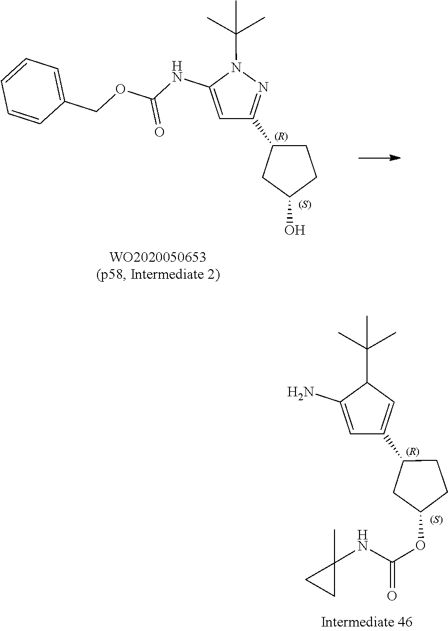

The title compound was prepared from benzyl (1-(tert-butyl)-3-((1S,3R)-3-hydroxycyclopentyl)-1H-pyrazol-5-yl)carbamate (WO2020050653; p58, Intermediate 2) using an analogous 4-part method as described for (Intermediate 6)

### Part 2. Synthesis of (1S,3R)-3-(3-(pyrazin-2-ylamino)-1H-pyrazol-5-yl)cyclopentyl(1-methylcyclopropyl)carbamate (Example 57)

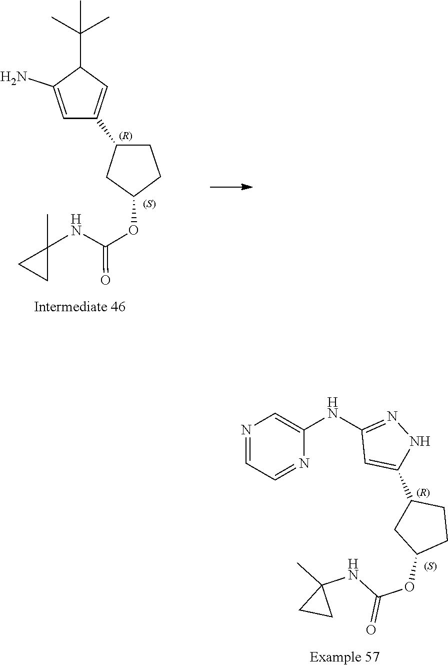

The title compound was prepared as a yellow solid (18.1 mg, 33% overall) from Intermediate 46 and 2-chloropyrazine using an analogous 2-part method as described for Intermediate 42 (Example 55, Parts 1 and 2). Prep-HPLC (Xtimate, 21.2×250 mm, 10 mm; 5-95% MeCN/H₂O (0.1% NH₄HCO₃). MS (ES+) C₁₇H₂₂N₆O₂ requires: 342, found: 343 [M+H]⁺. ¹H-NMR (400 MHz, DMSO-d₆) δ ppm 9.80 (br. s., 1H), 8.51 (s, 1H), 8.11 (s, 1H), 7.91-7.90 (m, 1H), 7.37 (s, 1H), 6.20 (s, 1H), 4.98 (s, 1H), 3.06-3.03 (m, 1H), 2.51-2.48 (m, 1H), 2.01-1.89 (m, 2H), 1.82-1.72 (m, 2H), 1.70-1.65 (m, 2H), 1.23 (s, 3H), 0.60-0.58 (m, 2H), 0.47-0.45 (m, 2H).

### Example 58 (1S,3R)-3-(3-((2-methoxypyrimidin-4-yl)amino)-1H-pyrazol-5-yl)cyclopentyl(1-methylcyclopropyl)carbamate

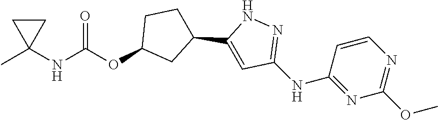

The title compound was prepared as a white solid (10.6 mg, 12.5% overall) from Intermediate 46 and 2-chloropyrazine using an analogous 2-Part method as described for Example 57. Prep-HPLC (Xtimate, 21.2×250 mm, 10 mm; 5-95% MeCN/H₂O (0.1% NH₄HCO₃). MS (ES+) C₁₈H₂₄N₆O₃ requires: 372, found: 373 [M+H]⁺. ¹H-NMR (400 MHz, DMSO-d₆) δ ppm 12.05 (br, s., 1H), 9.84 (s, 1H), 8.03 (d, J=5.6 Hz, 1H), 7.34 (s, 1H), 6.70 (br. s., 1H), 6.25 (br. s., 1H), 4.99 (s, 1H), 3.83 (s, 3H), 3.07-3.05 (m, 1H), 2.47-2.45 (m, 1H), 2.02-2.00 (m, 1H), 1.89-1.87 (m, 1H), 1.71-1.68 (m, 2H), 1.58-1.57 (m, 1H), 1.23 (s, 3H), 0.59-0.58 (m, 2H), 0.48-0.45 (m, 2H).

### Intermediate 51. Synthesis of tert-butyl 5-((tert-butoxycarbonyl)(5-cyanopyrazin-2-yl)amino)-3-((1S,3R)-3-((((2,5-dioxopyrrolidin-1-yl)oxy)carbonyl)oxy)cyclopentyl)-1H-pyrazole-1-carboxylate

### Step 1. Synthesis of benzyl (3-((1 S,3R)-3-hydroxycyclopentyl)-1H-pyrazol-5-yl)carbamate (Intermediate 47)

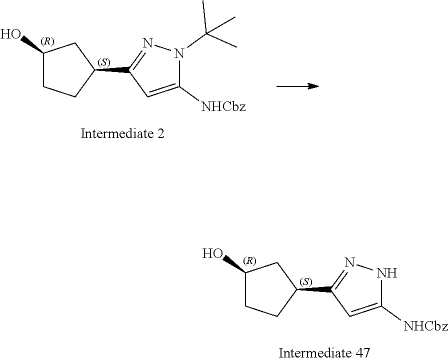

The reaction was carried out in 4 parallel batches. A mixture of Intermediate 2 (22.0 g, 59.8 mmol) in formic acid (450 mL) was stirred at 80° C. for 4 h under N₂. The reaction mixture was concentrated at 50° C. to remove most of the formic acid and the residue dissolved in MeOH (400 mL) and H₂O (180 mL). To this was added LiOH·H₂O (25.1 g, 598 mmol) and the reaction stirred at 20° C. for 2 h.

The 4 reactions were combined for work-up and the mixture poured into brine (2 L) and the aqueous phase extracted with EtOAc (3×800 mL). The combined organics were dried (Na₂SO₄) and concentrated under reduced pressure. The residue was purified by silica gel column chromatography (16-100% EtOAc/PE) to afford Intermediate 47 as a yellow gum (50.0 g, 683% yield). ¹H NMR: (400 MHz DMSO-d₆) δ: 11.89 (s, 1H), 9.83 (s, 1H), 7.47-7.29 (m, 5H) 6.11 (s, 1H), 5.12 (s, 2H), 4.65 (d, J=4.0 Hz, 1H), 4.21-4.13 (m, 1H), 2.96 (m, 1H), 2.32-2.21 (m, 1H), 1.97-1.87 (m, 1H), 1.80-1.66 (m, 2H), 1.65-1.56 (m, 1H), 1.54-1.42 (m, 1H).

### Step 2. Synthesis of (1R,3S)-3-(5-amino-1H-pyrazol-3-yl)cyclopentan-1-ol (Intermediate 48)

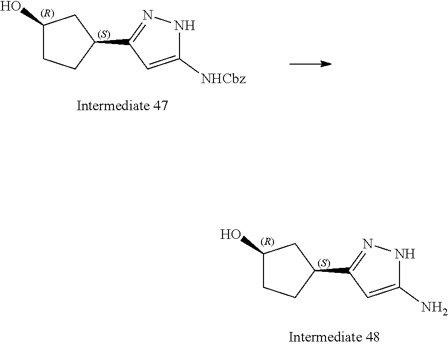

Intermediate 47 (25.0 g, 81.6 mmol) was added to Pd/C (26.1 g, 24.5 mmol, 10%), THE (250 mL) and EtOAc (250 mL) at 20° C. The suspension was degassed under vacuum and purged 3 times with H₂. The mixture was stirred under H₂ (15 psi) at 20° C. for 3 h. The reaction mixture was filtered under reduced pressure and filtrate evaporated to dryness in vacuo to afford Intermediate 48 as a black gum (26.0 g, crude) which was used without further purification. LCMS m/z=168 [M+H]⁺.

### Step 3. Synthesis of 5-((3-((1S,3R)-3-hydroxycyclopentyl)-1H-pyrazol-5-yl)amino)pyrazine-2-carbonitrile (Intermediate 49)

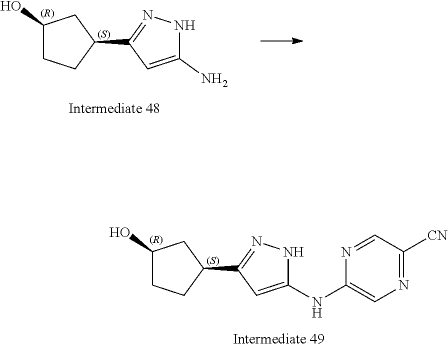

2 batches were carried out in parallel. 5-Chloropyrazine-2-carbonitrile (16.3 g, 116 mm0l) was added to a mixture of Intermediate 48 (13.0 g, 77.8 mmol) DIPEA (11.6 g, 89.4 mmol) in NMP (130 mL) at 20° C. The mixture was stirred at 80° C. for 16 h wider N₂. The 2 reactions were combined for work-up, cooled to 25° C. and water (150 mL) added and the mixture extracted with EtOAc (3×100 mL). The combined organics were washed with brine (3×100 mL), dried (Na₂SO₄) and evaporated to dryness in vacuo. The residue were purified by silica gel column chromatography (16-100% EtOAc/PE) to afford Intermediate 49 as a yellow solid (33.0 g, 77%).

### Step 4. Synthesis of tert-butyl 5-((tert-butoxycarbonyl)(5-cyanopyrazin-2-yl)amino)-3-((1S,3R)-3-hydroxycyclopentyl)-1H-pyrazole-1-carboxylate (intermediate 50)

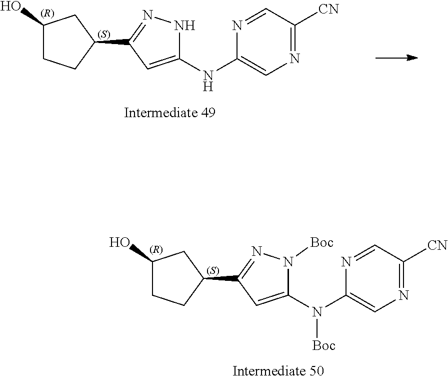

3 batches were carried out in parallel. TEA (12.1 g, 120 mmol) was added to a mixture of Intermediate 49 (11.0 g 39.9 mmol) and bis(tert-butoxycarbonyl)oxide (17.4 g, 79.9 mmol) in DCM (100 mL) at 20° C. and the mixture stirred at 30° C. for 24 h under N₂. The 3 reactions were combined and water (100 mL) added at 20° C. and the aqueous phase was extracted with DCM (3×150 mL). The combined organics were washed with brine (3×150 mL), dried (Na₂SO₄) and evaporated to dryness in vacuo. The residue was purified by silica gel column chromatography (16-100% EtOAc/PE) to afford Intermediate 50 as a yellow solid (15.0 g, 21.4%). LCMS m/z=471 [M+H]⁺.

### Step 4. Synthesis of tert-butyl 5-((tert-butoxycarbonyl)(5-cyanopyrazin-2-yl)amino)-3-((1S,3R)-3-((((2,5-dioxopyrrolidin-1-yl)oxy)carbonyl)oxy)cyclopentyl)-1H-pyrazole-1-carboxylate (Intermediate 51)

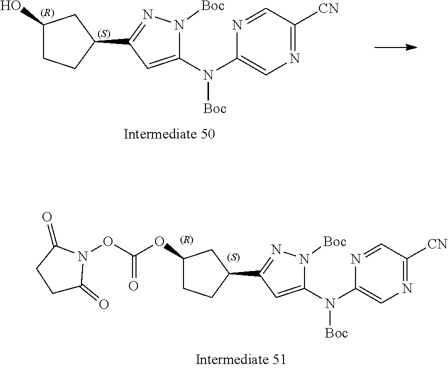

Bis(2,5-dioxopyrrolidin-1-yl) carbonate (15.1 g, 59.0 mmol) was added to a mixture of Intermediate 49 (15.0 g, 25.7 mmol) and TEA (7.79 g, 77.0 mmol) in MeCN (150 mL) at 0° C. The mixture was stirred at 0° C. for 0.5 h under N₂ and then at 20° C. for 6 h. The reaction mixture was diluted with water (150 mL) and extracted with EtOAc (3×100 mL). The combined organics were washed with brine (100 mL), dried (Na₂SO₄) and evaporated to dryness. The residue was purified by prep-HPLC (Phenomenex Luna C18; 400×100 mm, 15 μm; 20-60% MeCN/H₂O) to afford Intermediate 51 as a pink solid (10.2 g, 63.7%).

LCMS m/z=612 [M+H]⁺.

### Example 59-106

The title compounds were prepared from Intermediate 51 and the appropriate amine (R₁R₂NH) using a 2-step protocol as outlined below in the general scheme for the library

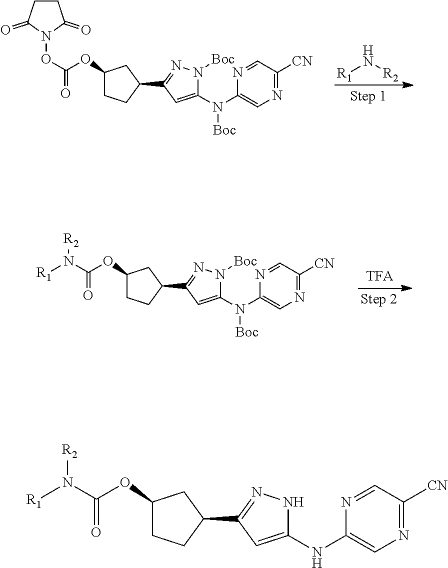

Step 1: To a solution of Intermediate 51 (79.43 mg, 0.13 mmol) and the appropriate amine (R₁R₂NH, 0.13 mmol) in THF (2 mL) was added DIPEA (0.116 ml, 0.65 mmol) and the mixture shaken at 30° C. for 5 h under N₂. The reaction mixture was evaporated to dryness using a Speedvac to give a residue.

Step 2: TFA (0.5 ml) was added to a solution of the residue of Step 1 in DCM (1.5 mL) and the mixture shaken at 20° C. for 2 h under N₂. The reaction mixture was evaporated to dryness using Speedvac. The residue was purified by prep-HPLC to give the title compounds.

HPLC-1: Xtimate C18, 150×25 mm, 5 mm; 0-100% MeCN/H₂O (0.225% HCO₂H). Gradient optimised for each sample; HPLC-2: Xtimate C18, 150×25 mm, 5 mm; 0-100% MeCN/H₂O (0.05% NH₄OH). Gradient optimised for each sample; HPLC-3: Xtimate C18 250×21.2 mm, 10 mm; 15-95% MeCN/H₂O (0.1% NH₄HCO₃); HPLC-4: Xtimate C18 250×21.2 mm, 10 mm; 15-35% MeCN/H₂O (0.1% HCO₂H); HPLC-5: Xtimate C18 250×21.2 mm, 10 mm; 25-45% MeCN/H₂O (10 mM NH₄HCO₃+NH₄OH)

| Example | Name, Structure, LCMS |
|---|---|
| 59 | (1R,3S)-3-(5-((5-cyanopyrazin-2-yl)amino)-1H-pyrazol-3- |
| | yl)cyclopentyl (3R*,4R*)-3,4-difluoropyrrolidine-1-carboxylate |
| | 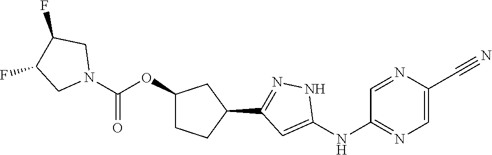 [SMILES: N#Cc1cnc(Nc2cc([C@H]3CC[C@@H](OC(=O)N4C[C@H](F)[C@@H](F)C4)C3)n[nH]2)cn1] |
| | R₁R₂NH: rel-(3R,4R)-3,4-difluoropyrrolidine hydrochloride |
| | HPLC-1; LCMS m/z = 404 [M + H]⁺ |
| 60 | (1R,3S)-3-(5-((5-cyanopyrazin-2-yl)amino)-1H-pyrazol-3- |
| | yl)cyclopentyl (3R*,4S*)-3,4-difluoropyrrolidine-1-carboxylate |
| | 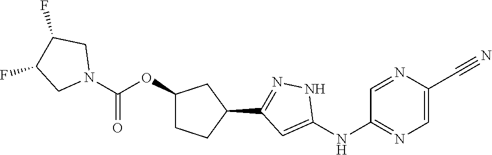 [SMILES: N#Cc1cnc(Nc2cc([C@H]3CC[C@@H](OC(=O)N4C[C@@H](F)[C@@H](F)C4)C3)n[nH]2)cn1] |
| | R₁R₂NH: rel-(3R,4S)-3,4-difluoropyrrolidine hydrochloride |
| | HPLC-1; LCMS m/z = 404 [M + H]⁺ |
| 61 | (1R,3S)-3-(5-((5-cyanopyrazin-2-yl)amino)-1H-pyrazol-3- |
| | yl)cyclopentyl cyclopropyl(methyl)carbamate |
| | 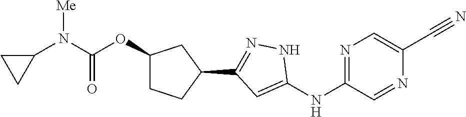 [SMILES: CN(C(=O)O[C@@H]1CC[C@H](c2cc(Nc3cnc(C#N)cn3)[nH]n2)C1)C1CC1] |
| | R₁R₂NH: N-methylcyclopropanamine |
| | HPLC-1; LCMS m/z = 368 [M + H]⁺ |
| 62 | (1R,3S)-3-(5-((5-cyanopyrazin-2-yl)amino)-1H-pyrazol-3- |
| | yl)cyclopentyl 3-methylazetidine-1-carboxylate |
| | 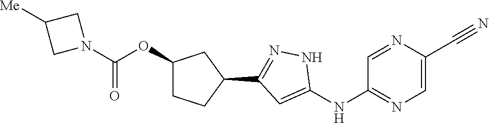 [SMILES: CC1CN(C(=O)O[C@@H]2CC[C@H](c3cc(Nc4cnc(C#N)cn4)[nH]n3)C2)C1] |
| | R₁R₂NH: 3-methylazetidine hydrochloride |
| | HPLC-1; LCMS m/z = 368 [M + H]⁺ |
| 63 | (1R,3S)-3-(5-((5-cyanopyrazin-2-yl)amino)-1H-pyrazol-3- |
| | yl)cyclopentyl 2-methylazetidine-1-carboxylate |
| | 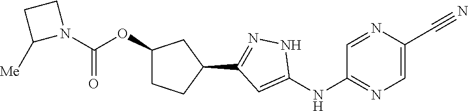 [SMILES: CC1CCN1C(=O)O[C@@H]1CC[C@H](c2cc(Nc3cnc(C#N)cn3)[nH]n2)C1] |
| | R₁R₂NH: (S)-2-methylazetidine |
| | HPLC-1; LCMS m/z = 368 [M + H]⁺ |
| 64 | (1R,3S)-3-(5-((5-cyanopyrazin-2-yl)amino)-1H-pyrazol-3- |
| | yl)cyclopentyl 3,3-dimethylazetidine-1-carboxylate |
| | 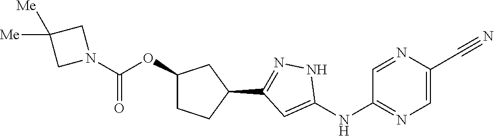 [SMILES: CC1(C)CN(C(=O)O[C@@H]2CC[C@H](c3cc(Nc4cnc(C#N)cn4)[nH]n3)C2)C1] |
| | R₁R₂NH: 3,3-dimethylazetidine |
| | HPLC-1; LCMS m/z = 382 [M + H]⁺ |
| 65 | (1R,3S)-3-(5-((5-cyanopyrazin-2-yl)amino)-1H-pyrazol-3- |
| | yl)cyclopentyl 3-methylpyrrolidine-1-carboxylate |
| | 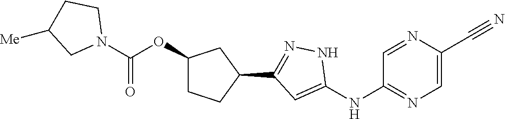 [SMILES: CC1CCN(C(=O)O[C@@H]2CC[C@H](c3cc(Nc4cnc(C#N)cn4)[nH]n3)C2)C1] |
| | R₁R₂NH: 3-methylpyrrolidine |
| | HPLC-1; LCMS m/z = 382 [M + H]⁺ |
| 66 | (1R,3S)-3-(5-((5-cyanopyrazin-2-yl)amino)-1H-pyrazol-3- |
| | yl)cyclopentyl 2,4-dimethylazetidine-1-carboxylate |
| | 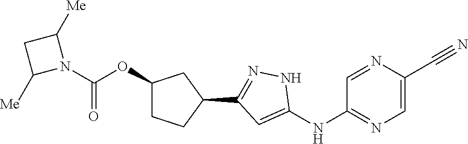 [SMILES: CC1CC(C)N1C(=O)O[C@@H]1CC[C@H](c2cc(Nc3cnc(C#N)cn3)[nH]n2)C1] |
| | R₁R₂NH: 2,4-dimethylazetidine hydrochloride |
| | HPLC-1; LCMS m/z = 382 [M + H]⁺ |
| 67 | (1R,3S)-3-(5-((5-cyanopyrazin-2-yl)amino)-1H-pyrazol-3- |
| | yl)cyclopentyl 2-methylpyrrolidine-1-carboxylate |
| | 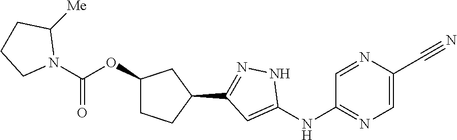 [SMILES: CC1CCCN1C(=O)O[C@@H]1CC[C@H](c2cc(Nc3cnc(C#N)cn3)[nH]n2)C1] |
| | R₁R₂NH: 2-methylpyrrolidine |
| | HPLC-1; LCMS m/z = 382 [M + H]⁺ |
| 68 | (1R,3S)-3-(5-((5-cyanopyrazin-2-yl)amino)-1H-pyrazol-3- |
| | yl)cyclopentyl 3-(trifluoromethyl)azetidine-1-carboxylate |
| | 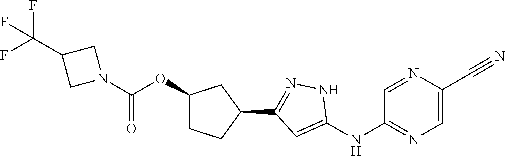 [SMILES: N#Cc1cnc(Nc2cc([C@H]3CC[C@@H](OC(=O)N4CC(C(F)(F)F)C4)C3)n[nH]2)cn1] |
| | R₁R₂NH: 3-(trifluoromethyl)azetidine |
| | HPLC-1; LCMS m/z = 422 [M + H]⁺ |
| 69 | (1R,3S)-3-(5-((5-cyanopyrazin-2-yl)amino)-1H-pyrazol-3- |
| | yl)cyclopentyl 2,2-dimethylpyrrolidine-1-carboxylate |
| | 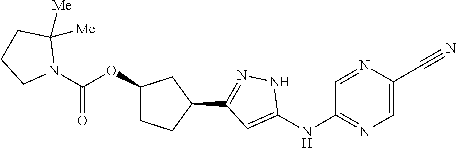 [SMILES: CC1(C)CCCN1C(=O)O[C@@H]1CC[C@H](c2cc(Nc3cnc(C#N)cn3)[nH]n2)C1] |
| | R₁R₂NH: 2,2-dimethylpyrrolidine |
| | HPLC-1; LCMS m/z = 396 [M + H]⁺ |
| 70 | (1R,3S)-3-(5-((5-cyanopyrazin-2-yl)amino)-1H-pyrazol-3- |
| | yl)cyclopentyl 4-methylpiperazine-1-carboxylate |
| | 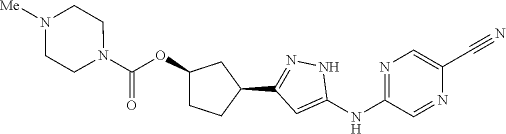 [SMILES: CN1CCN(C(=O)O[C@@H]2CC[C@H](c3cc(Nc4cnc(C#N)cn4)[nH]n3)C2)CC1] |
| | R₁R₂NH: 1-methylpiperazine |
| | HPLC-1; LCMS m/z = 397 [M + H]⁺ |
| 71 | (1R,3S)-3-(5-((5-cyanopyrazin-2-yl)amino)-1H-pyrazol-3- |
| | yl)cyclopentyl ethylcarbamate |
| | 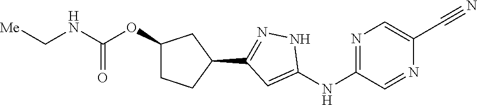 [SMILES: CCNC(=O)O[C@@H]1CC[C@H](c2cc(Nc3cnc(C#N)cn3)[nH]n2)C1] |
| | R₁R₂NH: ethanamine |
| | HPLC-1; LCMS m/z = 342 [M + H]⁺ |
| 72 | (1R,3S)-3-(5-((5-cyanopyrazin-2-yl)amino)-1H-pyrazol-3- |
| | yl)cyclopentyl cyclopropylcarbamate |
| | 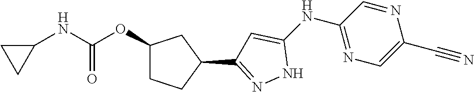 [SMILES: N#Cc1cnc(Nc2cc([C@H]3CC[C@@H](OC(=O)NC4CC4)C3)n[nH]2)cn1] |
| | R₁R₂NH: cyclopropanamine |
| | HPLC-2; LCMS m/z = 354 [M + H]⁺ |
| 73 | (1R,3S)-3-(5-((5-cyanopyrazin-2-yl)amino)-1H-pyrazol-3- |
| | yl)cyclopentyl (1-fluoropropan-2-yl)carbamate |
| | 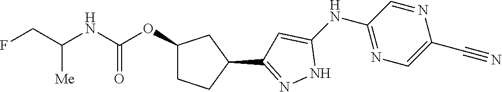 [SMILES: CC(CF)NC(=O)O[C@@H]1CC[C@H](c2cc(Nc3cnc(C#N)cn3)[nH]n2)C1] |
| | R₁R₂NH: 1-fluoropropan-2-amine hydrochloride |
| | HPLC-1; LCMS m/z = 374 [M + H]⁺ |
| 74 | (1R,3S)-3-(5-((5-cyanopyrazin-2-yl)amino)-1H-pyrazol-3- |
| | yl)cyclopentyl (cyclopropylmethyl)carbamate |
| | 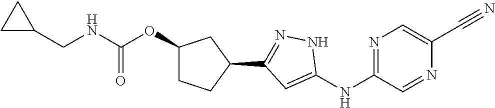 [SMILES: N#Cc1cnc(Nc2cc([C@H]3CC[C@@H](OC(=O)NCC4CC4)C3)n[nH]2)cn1] |
| | R₁R₂NH: cyclopropylmethanamine |
| | HPLC-2; LCMS m/z = 368 [M + H]⁺ |
| 75 | (1R,3S)-3-(5-((5-cyanopyrazin-2-yl)amino)-1H-pyrazol-3- |
| | yl)cyclopentyl (2-methylcyclopropyl)carbamate |
| | 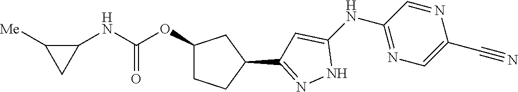 [SMILES: CC1CC1NC(=O)O[C@@H]1CC[C@H](c2cc(Nc3cnc(C#N)cn3)[nH]n2)C1] |
| | R₁R₂NH: 2-methylcyclopropan-1-amine hydrochloride |
| | HPLC-1; LCMS m/z = 368 [M + H]⁺ |
| 76 | (1R,3S)-3-(5-((5-cyanopyrazin-2-yl)amino)-1H-pyrazol-3- |
| | yl)cyclopentyl propylcarbamate |
| | 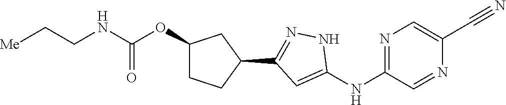 [SMILES: CCCNC(=O)O[C@@H]1CC[C@H](c2cc(Nc3cnc(C#N)cn3)[nH]n2)C1] |
| | R₁R₂NH: propan-1-amine |
| | HPLC-1; LCMS m/z = 356 [M + H]⁺ |
| 77 | (1R,3S)-3-(5-((5-cyanopyrazin-2-yl)amino)-1H-pyrazol-3- |
| | yl)cyclopentyl cyclobutylcarbamate |
| | 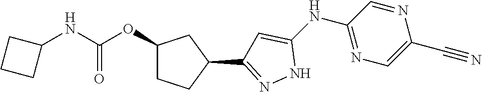 [SMILES: N#Cc1cnc(Nc2cc([C@H]3CC[C@@H](OC(=O)NC4CCC4)C3)n[nH]2)cn1] |
| | R₁R₂NH: cyclobutanamine |
| | HPLC-1; LCMS m/z = 368 [M + H]⁺ |
| 78 | (1R,3S)-3-(5-((5-cyanopyrazin-2-yl)amino)-1H-pyrazol-3- |
| | yl)cyclopentyl (1-cyclopropylethyl)carbamate |
| | 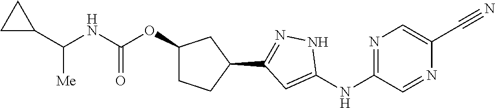 [SMILES: CC(NC(=O)O[C@@H]1CC[C@H](c2cc(Nc3cnc(C#N)cn3)[nH]n2)C1)C1CC1] |
| | R₁R₂NH: 1-cyclopropylethan-1-amine |
| | HPLC-1; LCMS m/z = 382 [M + H]⁺ |
| 79 | (1R,3S)-3-(5-((5-cyanopyrazin-2-yl)amino)-1H-pyrazol-3- |
| | yl)cyclopentyl (1-methylcyclobutyl)carbamate |
| | 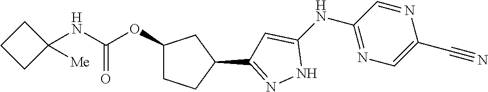 [SMILES: CC1(NC(=O)O[C@@H]2CC[C@H](c3cc(Nc4cnc(C#N)cn4)[nH]n3)C2)CCC1] |
| | R₁R₂NH: 1-methylcyclobutan-1-amine |
| | HPLC-1; LCMS m/z = 382 [M + H]⁺ |
| 80 | (1R,3S)-3-(5-((5-cyanopyrazin-2-yl)amino)-1H-pyrazol-3- |
| | yl)cyclopentyl cyclopentylcarbamate |
| | 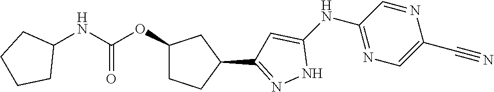 [SMILES: N#Cc1cnc(Nc2cc([C@H]3CC[C@@H](OC(=O)NC4CCCC4)C3)n[nH]2)cn1] |
| | R₁R₂NH: cyclopentanamine |
| | HPLC-1; LCMS m/z = 382 [M + H]⁺ |
| 81 | (1R,3S)-3-(5-((5-cyanopyrazin-2-yl)amino)-1H-pyrazol-3- |
| | yl)cyclopentyl (1,1,1-trifluoropropan-2-yl)carbamate |
| | 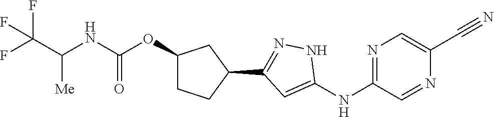 [SMILES: CC(NC(=O)O[C@@H]1CC[C@H](c2cc(Nc3cnc(C#N)cn3)[nH]n2)C1)C(F)(F)F] |
| | R₁R₂NH: (S)-1,1,1-trifluoropropan-2-amine |
| | HPLC-1; LCMS m/z 410 [M + H]⁺ |
| 82 | (1R,3S)-3-(5-((5-cyanopyrazin-2-yl)amino)-1H-pyrazol-3- |
| | yl)cyclopentyl (3,3,3-trifluoropropyl)carbamate |
| | 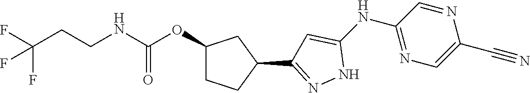 [SMILES: N#Cc1cnc(Nc2cc([C@H]3CC[C@@H](OC(=O)NCCC(F)(F)F)C3)n[nH]2)cn1] |
| | R₁R₂NH: 3,3,3-trifluoropropan-1-amine |
| | HPLC-1; LCMS m/z = 410 [M + H]⁺ |
| 83 | (1R,3S)-3-(5-((5-cyanopyrazin-2-yl)amino)-1H-pyrazol-3- |
| | yl)cyclopentyl (3,3-difluorobutan-2-yl)carbamate |
| | 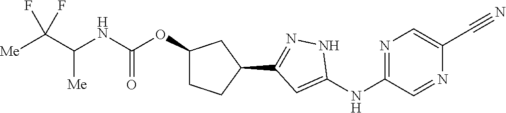 [SMILES: CC(NC(=O)O[C@@H]1CC[C@H](c2cc(Nc3cnc(C#N)cn3)[nH]n2)C1)C(C)(F)F] |
| | R₁R₂NH: (S)-3,3-difluorobutan-2-amine hydrochloride |
| | HPLC-1; LCMS m/z = 406 [M + H]⁺ |
| 84 | (1R,3S)-3-(5-((5-cyanopyrazin-2-yl)amino)-1H-pyrazol-3- |
| | yl)cyclopentyl ((1R,2SR)-2-methylcyclopentyl)carbamate |
| | 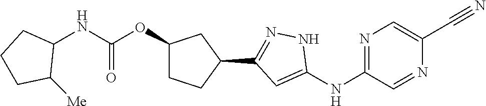 [SMILES: CC1CCCC1NC(=O)O[C@@H]1CC[C@H](c2cc(Nc3cnc(C#N)cn3)[nH]n2)C1] |
| | R₁R₂NH: 2-methylcyclopentan-1-amine hydrochloride |
| | HPLC-1; LCMS m/z = 396 [M + H]⁺ |
| 85 | (1R,3S)-3-(5-((5-cyanopyrazin-2-yl)amino)-1H-pyrazol-3- |
| | yl)cyclopentyl (2-methylbutyl)carbamate |
| | 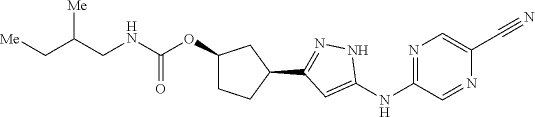 [SMILES: CCC(C)CNC(=O)O[C@@H]1CC[C@H](c2cc(Nc3cnc(C#N)cn3)[nH]n2)C1] |
| | R₁R₂NH: 2-methylbutan-1-amine |
| | HPLC-1; LCMS m/z = 384 [M + H]⁺ |
| 86 | (1R,3S)-3-(5-((5-cyanopyrazin-2-yl)amino)-1H-pyrazol-3- |
| | yl)cyclopentyl neopentylcarbamate |
| | 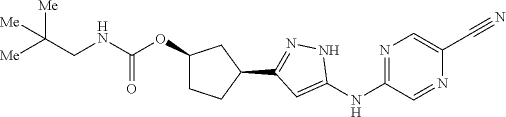 [SMILES: CC(C)(C)CNC(=O)O[C@@H]1CC[C@H](c2cc(Nc3cnc(C#N)cn3)[nH]n2)C1] |
| | R₁R₂NHL 2,2-dimethylpropan-1-amine |
| | HPLC-1; LCMS m/z = 384 [M + H]⁺ |
| 87 | (1R,3S)-3-(5-((5-cyanopyrazin-2-yl)amino)-1H-pyrazol-3- |
| | yl)cyclopentyl (4,4,4-trifluorobutan-2-yl)carbamate |
| | 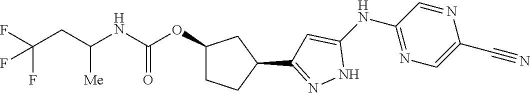 [SMILES: CC(CC(F)(F)F)NC(=O)O[C@@H]1CC[C@H](c2cc(Nc3cnc(C#N)cn3)[nH]n2)C1] |
| | R₁R₂NH: 4,4,4-trifluorobutan-2-amine |
| | HPLC-1; LCMS m/z = 424 [M + H]⁺ |
| 88 | (1R,3S)-3-(5-((5-cyanopyrazin-2-yl)amino)-1H-pyrazol-3- |
| | yl)cyclopentyl (1-(2,2,2-trifluoroethyl)cyclopropyl)carbamate |
| | 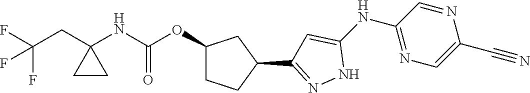 [SMILES: N#Cc1cnc(Nc2cc([C@H]3CC[C@@H](OC(=O)NC4(CC(F)(F)F)CC4)C3)n[nH]2)cn1] |
| | R₁R₂NH: 1-(2,2,2-trifluoroethyl)cyclopropan-1-amine hydrochloride |
| | HPLC-1; LCMS m/z = 436 [M = H]⁺ |
| 89 | (1R,3S)-3-(5-((5-cyanopyrazin-2-yl)amino)-1H-pyrazol-3- |
| | yl)cyclopentyl morpholine-4-carboxylate |
| | 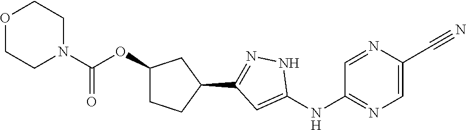 [SMILES: N#Cc1cnc(Nc2cc([C@H]3CC[C@@H](OC(=O)N4CCOCC4)C3)n[nH]2)cn1] |
| | R₁R₂NH: morpholine |
| | HPLC-1; LCMS m/z = 384 [M + H]⁺ |
| 90 | (1S,3S)-3-(5-((5-cyanopyrazin-2-yl)amino)-1H-pyrazol-3- |
| | yl)cyclopentyl 3-methoxyazetidine-1-carboxylate |
| | 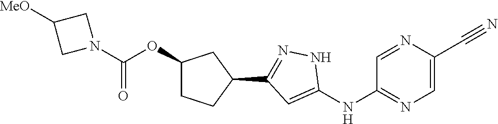 [SMILES: COC1CN(C(=O)O[C@@H]2CC[C@H](c3cc(Nc4cnc(C#N)cn4)[nH]n3)C2)C1] |
| | R₁R₂NH: 3-methoxyazetidine |
| | HPLC-1; LCMS m/z = 384 [M + H]⁺ |
| 91 | (1R,3S)-3-(5-((5-cyanopyrazin-2-yl)amino)-1H-pyrazol-3- |
| | yl)cyclopentyl 3-methylmorpholine-4-carboxylate |
| | 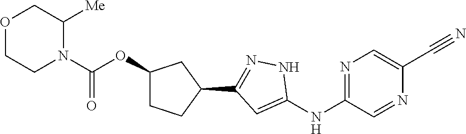 [SMILES: CC1COCCN1C(=O)O[C@@H]1CC[C@H](c2cc(Nc3cnc(C#N)cn3)[nH]n2)C1] |
| | R₁R₂NH: 3-methylmorpholine |
| | HPLC-1; LCMS m/z = 398 [M + H]⁺ |
| 92 | 1-(tert-butyl) 4-((1R,3S)-3-(5-((5-cyanopyrazin-2-yl)amino)-1H- |
| | pyrazol-3-yl)cyclopentyl) piperazine-1,4-dicarboxylate |
| | 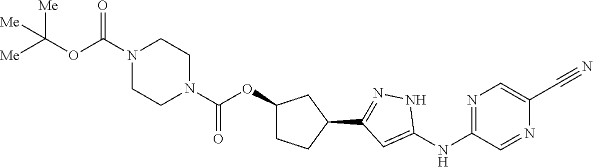 [SMILES: CC(C)(C)OC(=O)N1CCN(C(=O)O[C@@H]2CC[C@H](c3cc(Nc4cnc(C#N)cn4)[nH]n3)C2)CC1] |
| | R₁R₂NH: tert-butyl piperazine-1-carboxylate |
| | HPLC-1; LCMS m/z = 383 [M + H]⁺ |
| 93 | (1R,3S)-3-(5-((5-cyanopyrazin-2-yl)amino)-1H-pyrazol-3- |
| | yl)cyclopentyl (3-methyltetrahydrofuran-3-yl)carbamate |
| | 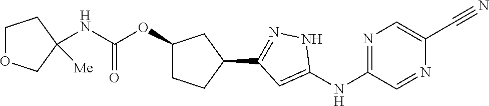 [SMILES: CC1(NC(=O)O[C@@H]2CC[C@H](c3cc(Nc4cnc(C#N)cn4)[nH]n3)C2)CCOC1] |
| | R₁R₂NH: 3-methyltetrahydrofuran-3-amine |
| | HPLC-1; LCMS m/z = 398 [M + H]⁺ |
| 94 | (1R,3S)-3-(5-((5-cyanopyrazin-2-yl)amino)-1H-pyrazol-3- |
| | yl)cyclopentyl (4-methyltetrahydro-2H-pyran-4-yl)carbamate |
| | 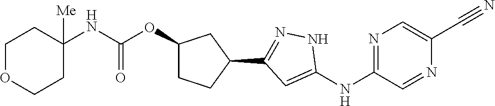 [SMILES: CC1(NC(=O)O[C@@H]2CC[C@H](c3cc(Nc4cnc(C#N)cn4)[nH]n3)C2)CCOCC1] |
| | R₁R₂NH: 4-methyltetrahydro-2H-pyran-4-amine |
| | HPLC-1; LCMS m/z = 412 [M + H]⁺ |
| 95 | (1R,3S)-3-(5-((5-cyanopyrazin-2-yl)amino)-1H-pyrazol-3- |
| | yl)cyclopentyl 3-cyanoazetidine-1-carboxylate |
| | 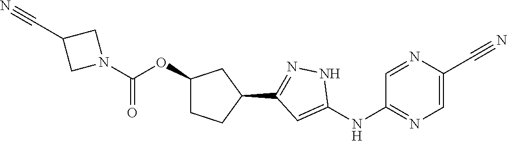 [SMILES: N#Cc1cnc(Nc2cc([C@H]3CC[C@@H](OC(=O)N4CC(C#N)C4)C3)n[nH]2)cn1] |
| | R₁R₂NH: azetidine-3-carbonitrile |
| | HPLC-1; LCMS m/z = 379 [M + H]⁺ |
| 96 | (1R,3S)-3-(5-((5-cyanopyrazin-2-yl)amino)-1H-pyrazol-3- |
| | yl)cyclopentyl 3-cyanopyrrolidine-1-carboxylate |
| | 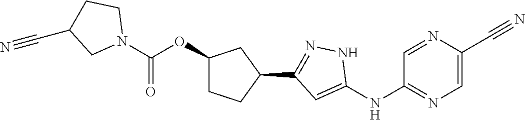 [SMILES: N#Cc1cnc(Nc2cc([C@H]3CC[C@@H](OC(=O)N4CCC(C#N)C4)C3)n[nH]2)cn1] |
| | R₁R₂NH: pyrrolidine-3-carbonitrile |
| | HPLC-1; LCMS m/z = 393 [M + H]⁺ |
| 97 | (1R,3S)-3-(5-((5-cyanopyrazin-2-yl)amino)-1H-pyrazole-3- |
| | yl)cyclopentyl ((R)-3,3-difluorobutan-2-yl)carbamate |
| | 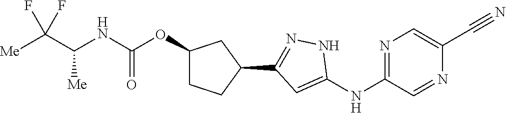 [SMILES: C[C@@H](NC(=O)O[C@@H]1CC[C@H](c2cc(Nc3cnc(C#N)cn3)[nH]n2)C1)C(C)(F)F] |
| | R₁R₂NH: (R)-3,3-difluorobutan-2-amine hydrochloride |
| | HPLC-3; White solid (6.3 mg, 15%); LCMS m/z = 406 [M + H]⁺; ¹H- |
| | NMR (400 MHz, DMSO-d₆) δ ppm 12.27 (s, 1H), 10.65 (s, 1H), |
| | 8.62 (s, 1H), 8.47 (s, 1H), 7.47 (d, J = 9.2 Hz, 1H), 6.35 (s, 1H), |
| | 5.04-5.02 (m, 1H), 3.92-3.88 (m, 1H), 3.12-3.08 (m, 1H), 2.07-1.92 |
| | (m, 5H), 1.76-1.71 (m, 1H), 1.54 (t, J = 18.8 Hz, 3H), 1.10 (d, J = 6.8 |
| | Hz, 3H). |
| 98 | (1R,3S)-3-(5-((5-cyanopyrazin-2-yl)amino)-1H-pyrazol-3- |
| | yl)cyclopentyl ((S)-3,3-difluorobutan-2-yl)carbamate |
| | 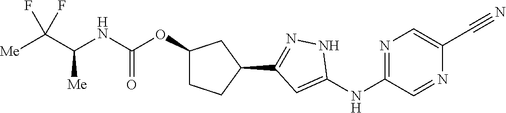 [SMILES: C[C@H](NC(=O)O[C@@H]1CC[C@H](c2cc(Nc3cnc(C#N)cn3)[nH]n2)C1)C(C)(F)F] |
| | R₁R₂NH: (S)-3,3-difluorobutan-2-amine hydrochloride |
| | HPLC-3; White solid (16.2 mg, 32%); LCMS m/z = 406 [M + H]⁺; |
| | ¹H-NMR (400 MHz, DMSO-d₆) δ ppm 12.29 (br. s., 1H), 10.65 (s, |
| | 1H), 8.62-8.60 (m, 1H), 8.49-8.45 (m, 1H), 7.45 (s, 1H), 6.35 (s, |
| | 1H), 5.03-4.99 (m, 1H), 3.92-3.80 (m, 1H), 3.13-3.09 (m, 1H), 2.06- |
| | 2.02 (m, 1H), 1.92-1.89 (m, 1H), 1.74-1.70 (m, 3H), 1.65-1.60 (m, |
| | 1H), 1.57-1.46 (m, 3H), 1.11-1.08 (m, 3H). |
| 100 | (1R,3S)-3-(5-((5-cyanopyrazin-2-yl)amino)-1H-pyrazol-3- |
| | yl)cyclopentyl ((R)-1-cyclopropylethyl)carbamate |
| | 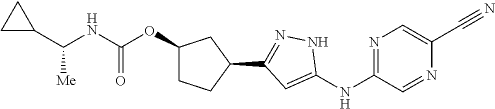 [SMILES: C[C@@H](NC(=O)O[C@@H]1CC[C@H](c2cc(Nc3cnc(C#N)cn3)[nH]n2)C1)C1CC1] |
| | R₁R₂NH: (1R)-1-cyclopropylethan-1-amine |
| | HPLC-3; White solid (27.1 mg, 45%); LCMS m/z = 382 [M + H]⁺; |
| | ¹H-NMR (400 MHz, DMSO-d₆) δ ppm 12.29 (br. s., 1H), 10.65 (s, |
| | 1H), 8.63 (s, 1H), 8.47 (br. s., 1H), 7.02 (d, J = 8.0 Hz, 1H), 6.35 (s, |
| | 1H), 5.00-4.98 (m, 1H), 3.12-3.06 (m, 1H), 3.00-2.95 (m, 1H), 2.07- |
| | 2.02 (m, 1H), 1.94-1.85 (m, 1H), 1.74-1.68 (m, 2H), 1.65-1.611 (m, |
| | 1H), 1.08 (d, J = 6.4 Hz, 3H), 0.82-0.78 (m, 1H), 0.35-0.07 (m, 4H). |
| 101 | (1R,3S)-3-(5-((5-cyanopyrazin-2-yl)amino)-1H-pyrazol-3- |
| | yl)cyclopentyl ((R)-1,1,1-trifluoropropan-2-yl)carbamate |
| | 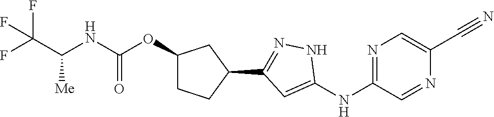 [SMILES: C[C@@H](NC(=O)O[C@@H]1CC[C@H](c2cc(Nc3cnc(C#N)cn3)[nH]n2)C1)C(F)(F)F] |
| | R₁R₂NH: (2R)-1,1,1-trifluoropropan-2-amine hydrochloride |
| | HPLC-3; White solid (30.3 mg, 50%); LCMS m/z = 410 [M + H]⁺; |
| | ¹H-NMR (400 MHz, DMSO-d₆) δ ppm 12.28 (s, 1H), 10.64 (s, 1H), |
| | 8.62 (s, 1H), 8.47 (s, 1H), 7.83 (d, J = 7.2 Hz, 1H), 6.35 (s, 1H), |
| | 5.07-5.04 (m, 1H), 4.29-4.24 (m, 1H), 3.12-3.08 (m, 1H), 2.51-2.50 |
| | (m, 1H), 2.02-1.92 (m, 2H), 1.77-1.63 (m, 3H), 1.23 (d, J = 6.4 Hz, |
| | 3H). |
| 102 | (1R,3S)-3-(5-((5-cyanopyrazin-2-yl)amino)-1H-pyrazol-3- |
| | yl)cyclopentyl ((S)-1-cyclopropylethyl)carbamate |
| | 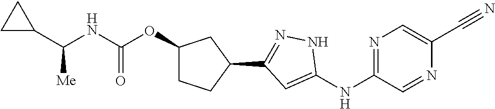 [SMILES: C[C@H](NC(=O)O[C@@H]1CC[C@H](c2cc(Nc3cnc(C#N)cn3)[nH]n2)C1)C1CC1] |
| | R₁R₂NH: (S)-1-cyclopropylethan-1-amine hydrochloride |
| | HPLC-3; White solid (33 mg, 56%); LCMS m/z = 382 [M + H]⁺; ¹H- |
| | NMR (400 MHz, DMSO-d₆) δ ppm 12.29 (br. s., 1H), 10.65 (s, 1H), |
| | 8.63 (s, 1H), 8.47 (br. s., 1H), 7.02 (d, J = 8.0 Hz, 1H), 6.35 (s, 1H), |
| | 5.00-4.98 (m, 1H), 3.12-3.06 (m, 1H), 3.00-2.95 (m, 1H), 2.07-2.02 |
| | (m, 1H), 1.94-1.85 (m, 1H), 1.74-1.68 (m, 2H), 1.65-1.611 (m, 1H), |
| | 1.08 (d, J = 6.4 Hz, 3H), 0.82-0.78 (m, 1H), 0.39-0.35 (m, 1H), |
| | 0.33-0.27 (m, 1H), 0.25-0.19 (m, 1H), 0.11-0.07 (m, 1H). |
| 103 | (1R,3S)-3-(5-((5-cyanopyrazin-2-yl)amino)-1H-pyrazol-3- |
| | yl)cyclopentyl ((S)-1,1,1-trifluoropropan-2-yl)carbamate |
| | 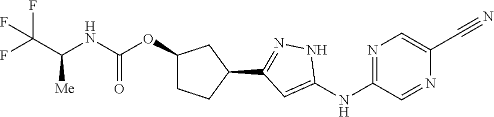 [SMILES: C[C@H](NC(=O)O[C@@H]1CC[C@H](c2cc(Nc3cnc(C#N)cn3)[nH]n2)C1)C(F)(F)F] |
| | R₁R₂NH: (2S)-1,1,1-trifluoropropan-2-amine |
| | HPLC-4; White solid (12.6 mg, 37%); LCMS m/z = 410 [M + H]⁺; |
| | ¹H-NMR (400 MHz, DMSO-d₆) δ ppm 12.32 (s, 1H), 10.65 (s, 1H), |
| | 8.62 (s, 1H), 8.47 (s, 1H), 7.84 (d, J = 8.8 Hz, 1H), 6.35 (s, 1H), |
| | 5.07-5.05 (m, 1H), 4.30-4.25 (m, 1H), 3.12-3.09 (m, 1H), 2.06-2.02 |
| | (m, 1H), 1.94-1.92 (m, 1H), 1.79-1.63 (m, 3H), 1.22 (d, J = 7.2 Hz, |
| | 3H). |
| 104 | (1R,3S)-3-(5-((5-cyanopyrazin-2-yl)amino)-1H-pyrazol-3- |
| | yl)cyclopentyl (2R,4R)-2,4-dimethylazetidine-1-carboxylate |
| | 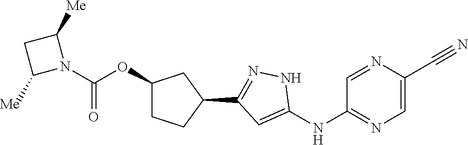 [SMILES: C[C@@H]1C[C@@H](C)N1C(=O)O[C@@H]1CC[C@H](c2cc(Nc3cnc(C#N)cn3)[nH]n2)C1] |
| | R₁R₂NH: (2R,4R)-2,4-dimethylazetidine hydrochloride |
| | HPLC-3; White solid (23.4 mg, 44%); LCMS m/z = 382 [M + H]⁺; |
| | ¹H-NMR (400 MHz, DMSO-d₆) δ ppm 12.30 (s, 1H), 10.65 (s, 1H), |
| | 8.61 (s, 1H), 8.45 (s, 1H), 6.34 (s, 1H), 5.03-5.01 (m, 1H), 4.22-4.18 |
| | (m, 2H), 3.14-3.09 (m, 1H), 2.47-2.40 (m, 1H), 2.08-2.01 (m, 1H), |
| | 1.90-1.86 (m, 3H), 1.73-1.64 (m, 3H), 1.29-1.24 (m, 6H). |
| 105 | (1R,3S)-3-(5-((5-cyanopyrazin-2-yl)amino)-1H-pyrazol-3- |
| | yl)cyclopentyl (2S,4R)-2,4-dimethylazetidine-1-carboxylate |
| | 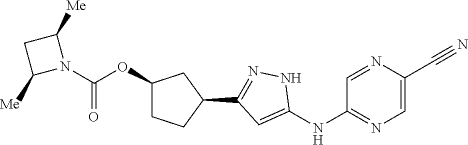 [SMILES: C[C@@H]1C[C@H](C)N1C(=O)O[C@@H]1CC[C@H](c2cc(Nc3cnc(C#N)cn3)[nH]n2)C1] |
| | R₁R₂NH: (2R,4S)-2,4-dimethylazetidine hydrochloride |
| | HPLC-5; White solid (9.6 mg, 36%); LCMS m/z = 382 [M + H]⁺; ¹H- |
| | NMR (400 MHz, DMSO-d₆) δ ppm 12.27 (br. s., 1H), 10.65 (s, 1H), |
| | 8.61 (s, 1H), 8.45 (s, 1H), 6.35 (s, 1H), 5.05-5.00 (m, 1H), 4.09-4.02 |
| | (m, 2H), 3.17-3.08 (m, 1H), 2.46-2.41 (m, 2H), 2.07-2.04 (m, 1H), |
| | 1.90-1.83 (m, 1H), 1.79-1.73 (m, 2H), 1.71-1.65 (m, 1H), 1.38-1.32 |
| | (m, 1H), 1.26 (d, J = 6.0 Hz, 6H). |
| 106 | (1R,3S)-3-(5-((5-cyanopyrazin-2-yl)amino)-1H-pyrazol-3- |
| | yl)cyclopentyl (2S,4S)-2,4-dimethylazetidine-1-carboxylate |
| | 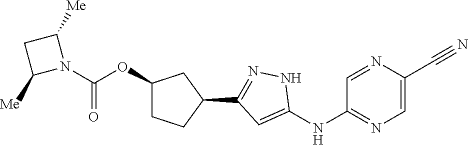 [SMILES: C[C@H]1C[C@H](C)N1C(=O)O[C@@H]1CC[C@H](c2cc(Nc3cnc(C#N)cn3)[nH]n2)C1] |
| | R₁R₂NH: (2S,4S)-2,4-dimethylazetidine hydrochloride |
| | HPLC-4; White solid (15.3 mg, 33%); LCMS m/z = 382 [M + H]⁺; |
| | ¹H-NMR (400 MHz, DMSO-d₆) δ ppm 12.85 (br. s., 1H), 10.65 (s, |
| | 1H), 8.62 (s, 1H), 8.45 (s, 1H), 6.35 (s, 1H), 5.01-4.99 (m, 1H), |
| | 4.22-4.20 (m, 2H), 3.13-3.09 (m, 1H), 2.50-2.45 (m, 1H), 2.08-2.04 |
| | (m, 1H), 1.91-1.90 (m, 3H), 1.88-1.87 (m, 2H), 1.77-1.75 (m, 1H), |
| | 1.26-1.22 (m, 6H). |

### Example 107 and 108. Synthesis of (1R,3S)-3-(5-((5-cyanopyrazin-2-yl)amino)-1H-pyrazol-3-yl)cyclopentyl((S)-3-methyltetrahydrofuran-3-yl)carbamate or (1R,3S)-3-(5-((5-cyanopyrazin-2-yl)amino)-1H-pyrazol-3-yl)cyclopentyl((R)-3-methyltetrahydrofuran-3-yl)carbamate

### Step 1. Synthesis of tert-butyl 5-((tert-butoxycarbonyl)(5-cyanopyrazin-2-yl)amino)-3-((1S,3R)-3-(((3-methyltetrahydrofuran-3-yl)carbamoyl)oxy)cyclopentyl)-71H-pyrazole-1-carboxylate (Intermediate XX)

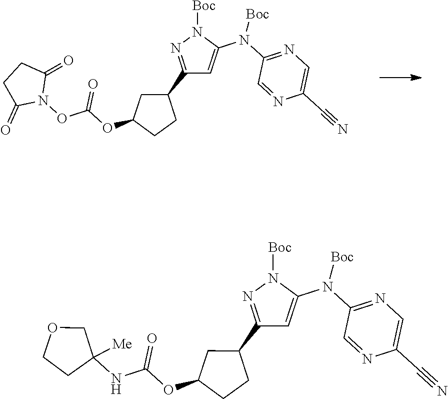 [SMILES: CC(C)(C)OC(=O)N(c1cnc(C#N)cn1)c1cc([C@H]2CC[C@@H](OC(=O)ON3C(=O)CCC3=O)C2)nn1C(=O)OC(C)(C)C ; CC1(NC(=O)O[C@@H]2CC[C@H](c3cc(N(C(=O)OC(C)(C)C)c4cnc(C#N)cn4)n(C(=O)OC(C)(C)C)n3)C2)CCOC1]

A solution of tert-butyl 5-((tert-butoxy carbonyl)(5-cyanopyrazin-2-yl)amino)-3 ((1S,3R)-3-((((2,5-dioxopyrrolidin-1-yl)oxy)carbonyl)oxy)cyclopentyl)-1H-pyrazole-1-carboxylate (Intermediate 51, 150 mg, 0.245 mmol), DIPEA (94.5 mg, 0.098 mmol) and 3-methyloxolan-3-amine (248 mg, 0.736 mmol) in THF (3 mL) was stirred for 3 h at rt under N₂. The reaction mixture was evaporated to dryness and the residue was purified by flash chromatography (SiO2, 25% EtOAc/PE) to give the title compound as a yellow oil (120 mg, 82%). LCMS m/z=498 [M−Boc]⁺.

### Step 2. Synthesis of (1R,3S)-3-(5-((5-cyanopyrazin-2-yl)amino)-1H-pyrazol-3-yl)cyclopentyl((R)-3-methyltetrahydrofuran-3-yl)carbamate and (1R,3S)-3-(5-((5-cyanopyrazin-2-yl)amino)-1H-pyrazol-3-yl)cyclopentyl((S)-3-methyltetrahydrofuran-3-yl)carbamate

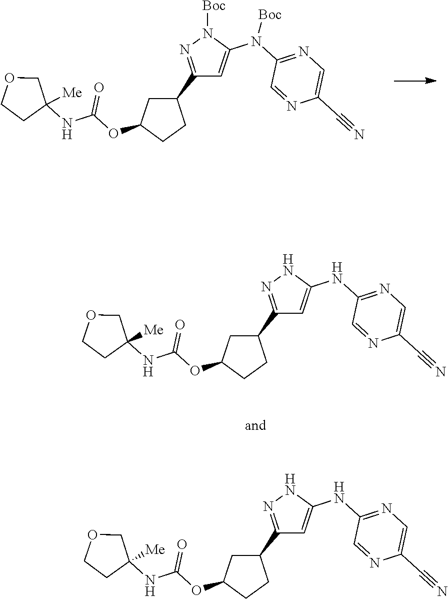 [SMILES: CC1(NC(=O)O[C@@H]2CC[C@H](c3cc(N(C(=O)OC(C)(C)C)c4cnc(C#N)cn4)n(C(=O)OC(C)(C)C)n3)C2)CCOC1 ; C[C@@]1(NC(=O)O[C@@H]2CC[C@H](c3cc(Nc4cnc(C#N)cn4)[nH]n3)C2)CCOC1 ; C[C@]1(NC(=O)O[C@@H]2CC[C@H](c3cc(Nc4cnc(C#N)cn4)[nH]n3)C2)CCOC1]

To a solution of tert-butyl 5-((tert-butoxycarbonyl) (5-cyanopyrazin-2-yl)amino)-3-((1S,3R)-3-(((3-methyltetrahydrofuran-3-yl)carbamoyl)oxy)cyclopentyl)-1H-pyrazole-1-carboxylate (Step 1, 100 mg, 0.167 mmol) in DCM (3 mL) was added TFA (1 mL) at rt and the resulting mixture stirred for 2 h at 50° C. under N₂. The reaction mixture was evaporated to dryness in vacuo and the residue was purified by prep-HPLC-4 to obtain (1R,3S)-3-(5-((5-cyanopyrazin-2-yl)amino)-1H-pyrazol-3-yl)cyclopentyl(3-methyltetrahydrofuran-3-yl)carbamate as a white solid (60 mg, 90%). The racemate was separated by chiral-SFC (Diacel IC-20, 20×250 mm, 10 mm; 45% IPA (0.5% NH₃ (7 M in MeOH)) in CO₂ to afford:

Peak 1, Example 107: (1R,3S)-3-(5-((5-cyanopyrazin-2-yl)amino)-1H-pyrazol-3-yl)cyclopentyl((R)-3-methyltetrahydrofuran-3-yl)carbamate or (1R,3S)-3-(5-((5-cyanopyrazin-2-yl)amino)-1H-pyrazol-3-yl)cyclopentyl((S)-3-methyltetrahydrofuran-3-yl)carbamate (21.1 nip, 35%). LCMS m/z=398 [M+H]⁺; ¹H-NMR (400 MHz, DMSO-d₆) δ ppm 12.29 (s, 1H), 10.65 (s, 1H), 8.63 (s, 1H), 8.45 (s, 1H), 7.31 (s, 1H), 6.44 (s, 1H), 4.99-4.88 (m, 1H), 3.75-3.71 (m, 3H), 3.44-3.42 (m, 1H), 3.25-3.20 (m, 1H) 2.20-2.15 (m, 1H), 2.10-2.02 (m, 1H), 1.85-1.78 (m, 1H), 1.73-1.30 (m, 5H), 1.32 (s, 31H).

Peak 2, Example 108: (1R,3S)-3-(5-((5-cyanopyrazin-2-yl)amino)-1H-pyrazol-3-yl)cyclopentyl((R)-3-methyltetrahydrofuran-3-yl)carbamate or (1R,3S)-3-(5-((5-cyanopyrazin-2-yl)amino)-1H-pyrazol-3-yl)cyclopentyl((S)-3-methyltetrahydrofuran-3-yl)carbamate (22.2 nip, 37%). LCMS n/z 398 [M+H]⁺; ¹H-NMR (400 MHz, DMSO-d₆) δ ppm 12.39 (s, 1H), 10.64 (s, 1H), 8.62 (s, 1H), 8.46 (s 1H), 7.28 (s 1H), 6.44 (s, 1H), 4.99-4.88 (m, 1H), 3.75-3.71 (m, 3H), 3.45-3.43 (m, 1H), 3.26-3.14 (m, 1H) 2.24-2.16 (m, 1H), 2.08-2.02 (m, 1H), 1.83-1.78 (m, 1H), 1.71-1.33 (m, 5H), 1.29 (s, 3H).

### Biological Example 1. Biochemical CDK Inhibition Assays

Inhibitory effects of the compounds of the disclosure were measured in biochemical assays that measure the enzymatic phosphorylation activity of CDK enzyme in complex of Cyclin proteins phosphorylates 7.5 micromolar fluorescently labelled peptide substrate, 5-FAM-QSPKKG-CONH2, (FL-Peptide 18, Perkin Elmer, 760362) in the presence of adenosine-5′-triphosphate (ATP) and varying concentrations of the test compound in 100 mM 2-[4-(2-hydroxyethyl)piperazin-1-yl]ethanesulfonic acid (HEPES), pH 7.5, 10 mM MgCl₂, 0.015% Brij-35, 1 mM dithiothreitol (DTT), 1.0% dimethylsulfoxide (DMSO). Assays were performed at 1.0 mM ATP or at ATP Km of the CDK enzymes in complex with Cyclin proteins. Reactions proceeded until between 10% to 20% total peptides were phosphorylated at room temperature (25° C.) and were terminated with 35 mM 2,2′,2″,2′″-(ethane-1,2-diyldinitrilo)tetraacetic acid (EDTA). Product was detected using the Caliper mobility shift detection method where the phosphorylated peptide (product) and substrate were electrophoretically separated and measured. Percent activity was plotted against log concentration of compound and points to generate an apparent IC₅₀. The following CDK enzymes in complex with different cyclin proteins were used in these assays:
CDK1/Cyclin B1, GST-tag (BPS, 40454), 1.5 nM used in the assay
CDK2/Cyclin E (Eurofins, 14-475), 1.25 nM used in the assays

Biological assay data of the test compounds are provided in Table 1 below. For inhibitory activity against CDK2/Cyclin B mutant, the following designations are used: ≤10 nM=A; >10-20 nM=B; >20-30 nM=C; >30-100 nM=D and >100=E. For inhibition CDK1/Cyclin B1, GST-tag: ≥500 nM=A; 100-<500 nM=B; <100 nM=C.

TABLE 1
Compound assay data

| | CDK2/cyclin E1 | CDK1/cyclin B1 |
|---|---|---|
| | IC50 | IC50 |
| no. | (nM) | (nM) |
| 1 | A | B |
| 2 | A | C |
| 3 | A | C |
| 4 | A | A |
| 5 | A | B |
| 6 | A | A |
| 7 | A | C |
| 8 | A | B |
| 9 | A | C |
| 10 | A | C |
| 11 | A | C |
| 12 | A | B |
| 13 | B | A |
| 14 | A | A |
| 15 | A | C |
| 16 | A | C |
| 17 | A | B |
| 18 | B | A |
| 19 | A | B |
| 20 | A | A |
| 21 | A | B |
| 22 | A | B |
| 23 | A | C |
| 24 | A | B |
| 25 | A | B |
| 26 | A | B |
| 27 | A | B |
| 28 | A | B |
| 29 | A | C |
| 30 | A | B |
| 31 | A | C |
| 32 | A | A |
| 33 | A | C |
| 34 | A | B |
| 35 | A | A |
| 36 | A | C |
| 37 | A | B |
| 38 | B | A |
| 39 | A | B |
| 40 | B | A |
| 41 | A | A |
| 42 | C | A |
| 43 | A | B |
| 44 | A | C |
| 45 | A | A |
| 46 | A | B |
| 47 | A | A |
| 48 | A | B |
| 49 | A | C |
| 50 | A | C |
| 51 | A | C |
| 52 | A | C |
| 53 | A | B |
| 54 | A | C |
| 55 | A | C |
| 56 | A | C |
| 57 | A | C |
| 58 | A | C |

### Biological Example 2. NanoBRET Cellular Binding Assays

Cellular target engagement, or cellular binding of the testing compounds were measured in NanoBRET assay s, which is based on binding competition between the testing compounds and a bioluminescent tracer in human embryonic kidney cells (HEK-293 cell line). In the assay, HEK-293 cells were cultured to appropriate confluence before being transiently transfected with a mix of CDK2-NanoLuc(R) Fusion Vector (Promega, NV2781) and CCNE1 expression vector (Promega, NV2641) for CDK2 NanoBRET assays, or CDK1-NanoLuc(R) Fusion Vector (Promega, NV2701) and CCNB1 expression vector (Promega, NV2601) for CDK1 NanoBRET assay, by using lipid:DNA complexes formed with FuGENE HD transfection agent (Promega, E2311). The transfected cells were cultured overnight in 1% FBS Opti-MEM media to allow the expression to fully occur. Once the expression fully occurred, varying concentrations of the test compounds were added to the cells, before a cell-permeable, fluorescent tracer (tracer K10 from Promega, N2840) was added to achieve final tracer concentration of 0.5 μM. After the addition of Nano-Glo substrate (part of a kit, Promega, N2840) and Extracellular NanoLuc inhibitor (part of a kit, Promega, N2840) 2 hrs after tracer addition, the cellular engagement of the tracer and the competition with testing compounds to the target protein was assessed based on the resulting bioluminescence resonance energy transfer (BRET) signal. BRET ratio was generated by diving the acceptor emission signal (610 nm) by the donor emission signal (450 nm) for each sample. The raw BRET ratio was converted to milliBRET ratio by multiplying each raw value by 1000. The milliBRET ratio was then used directly for non-linear IC₅₀ fitting with n:=1 at each concentration of the test compounds. All IC50 curves were calculated using a 4 parameter logistic nonlinear regression model.

Biological assay data of the test compounds are provided in Table 2 below. For inhibitory activity against CDK2/Cyclin E mutant, the following designations are used: ≤20 nM=A; >20-30 nM=B; >30-100 nM=C; and >100 nM=D. For inhibition CDK1/Cyclin B1, GST-tag: ≥500 nM=A; 100-<500 nM=B; <100 nM=C

TABLE 2
Compound assay data

| | NanoBRET CDK2/cyclin | NanoBRET |
|---|---|---|
| | E1 | CDK1/cyclin B1 |
| | IC50 | IC50 |
| no. | (nM) | (nM) |
| 59 | A | B |
| 60 | A | C |
| 61 | A | A |
| 62 | A | B |
| 63 | A | C |
| 64 | A | A |
| 65 | A | B |
| 66 | A | C |
| 67 | A | C |
| 68 | A | B |
| 69 | A | C |
| 70 | A | A |
| 71 | A | B |
| 72 | A | B |
| 73 | A | B |
| 74 | A | B |
| 75 | A | A |
| 76 | A | B |
| 77 | A | A |
| 78 | A | B |
| 79 | A | C |
| 80 | A | A |
| 81 | C | A |
| 82 | A | B |
| 83 | A | A |
| 84 | A | A |
| 85 | A | B |
| 86 | A | B |
| 87 | A | C |
| 88 | A | B |
| 89 | A | A |
| 90 | A | A |
| 91 | A | A |
| 92 | C | B |
| 93 | A | B |
| 94 | A | A |
| 95 | B | B |
| 96 | A | B |
| 97 | B | A |
| 98 | A | A |
| 100 | A | B |
| 101 | C | A |
| 102 | A | B |
| 103 | B | A |
| 104 | C | A |
| 105 | A | B |
| 106 | A | C |

1. A compound of Formula (I-1), 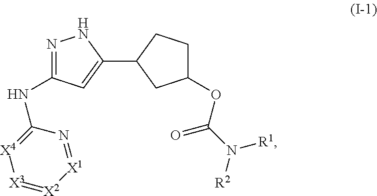
or a pharmaceutically acceptable salt thereof, wherein
R¹ is C₁-C₆alkyl, C₃-C₆cycloalkyl, or 4 to 6 membered heterocyclyl, wherein the C₁-C₆alkyl is optionally substituted with 1 to 4 groups independently selected from halo and C₃-C₆cycloalkyl, wherein the cycloalkyl or heterocyclyl is optionally substituted with 1 to 4 groups independently selected from halo and C₁-C₄alkyl optionally substituted with 1 to 4 halo, wherein the heterocyclyl includes 1 or 2 heteroatoms independently selected from O, N, or S;
R² is H or C₁-C₆ alkyl, or
R¹ and R², taken together with the nitrogen atom to which they are attached, form a 4 to 6 membered heterocyclyl, wherein the heterocyclyl optionally includes 1 or 2 heteroatoms independently selected from O, N, or S in addition to the nitrogen atom attached to R¹ and R², and is optionally substituted with 1 to 4 groups independently selected from the group consisting of halo, CO₂R^{d}, CN, C₁-C₄alkyl and C₁-C₄alkoxy, wherein the C₁-C₄alkyl and C₁-C₄alkoxy are each optionally substituted with 1 to 4 halo,
X¹ is N or CR³;
X² is N or CR³;
X³ is N or CR³;
X⁴ is N or CR²; wherein at least 2 of X¹, X², X³, and X⁴ are CR³;
R³, independently for each occurrence, is selected from the group consisting of H, halo, CN, OH, NR^{a}R^{b}, SO_{w}R^{c}, C₁-C₄alkyl, C₁-C₄alkoxy, and C₃-C₆cycloalkyl, wherein the alkyl or alkoxy is optionally substituted with 1 to 4 groups independently selected from halo, OR^{3a}, and C₃-C₆cycloalkyl, and the C₃-C₆cycloalkyl is optionally substituted with 1 to 4 groups independently selected from halo and OH;
w is 0, 1, or 2;
R^{a} is independently for each occurrence H or C₁-C₄alkyl optionally substituted by one, two or three halos;
R^{b} is independently for each occurrence H or C₁-C₄alkyl optionally substituted by one, two or three halos;
R^{c} is independently for each occurrence C₁-C₄alkyl optionally substituted by one, two or three halos;
R^{d} is independently for each occurrence H or C₁-C₄alkyl optionally substituted by one, two or three halos; and
R^{3a} for each occurrence is H or C₁₋₄alkyl optionally substituted by one, two or three halos.

2. A compound of Formula (I), 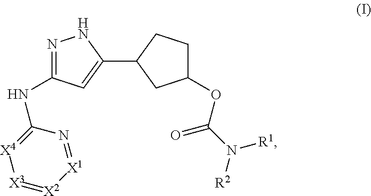
or a pharmaceutically acceptable salt thereof, wherein
R¹ is C₁-C₆alkyl, C₃-C₆cycloalkyl, or 4 to 6 membered heterocyclyl, wherein the cycloalkyl or heterocyclyl is optionally substituted with 1 to 4 groups selected from halo and C₁-C₄alkyl optionally substituted with 1 to 4 halo, wherein the heterocyclyl includes 1 or 2 heteroatoms selected from O, N, or S;
R² is H or C₁-C₆ alkyl, or
R¹ and R², taken together with the nitrogen atom to which they are attached, form a 4 to 6 membered heterocyclyl, optionally substituted with 1 to 4 groups selected from halo or C₁-C₄alkyl,
X¹ is N or CR³;
X² is N or CR³;
X³ is Nor CR³;
X⁴ is N or CR³; wherein at least 2 of X¹, X², X³, and X⁴ are CR³;
R³, independently for each occurrence is selected from the group consisting of H, halo, CN, OH, NR^{a}R^{b}, SO_{w}R^{c}, C₁-C₄alkyl, C₁-C₄alkoxy, and C₃-C₆cycloalkyl, wherein the alkyl or alkoxy is optionally substituted with 1 to 4 groups independently selected from halo, OR^{3a}, and C₃-C₆cycloalkyl, and the C₃-C₆cycloalkyl is optionally substituted with 1 to 4 groups independently selected from halo and OH;
w is 0, 1, or 2;
R^{a} is independently for each occurrence H or C₁-C₄alkyl optionally substituted by one, two or three halos;
R^{b} is independently for each occurrence H or C₁-C₄alkyl optionally substituted by one, two or three halos;
R^{c} is independently for each occurrence C₁-C₄alkyl optionally substituted by one, two or three halos; and
R^{3a} for each occurrence is H or C₁₋₄alkyl optionally substituted by one, two or three halos.

3. The compound of claim 1 or 2, wherein the compound is selected from the group consisting of: Formula IIA, IIB, IIC, IID, IIE, and IIF: 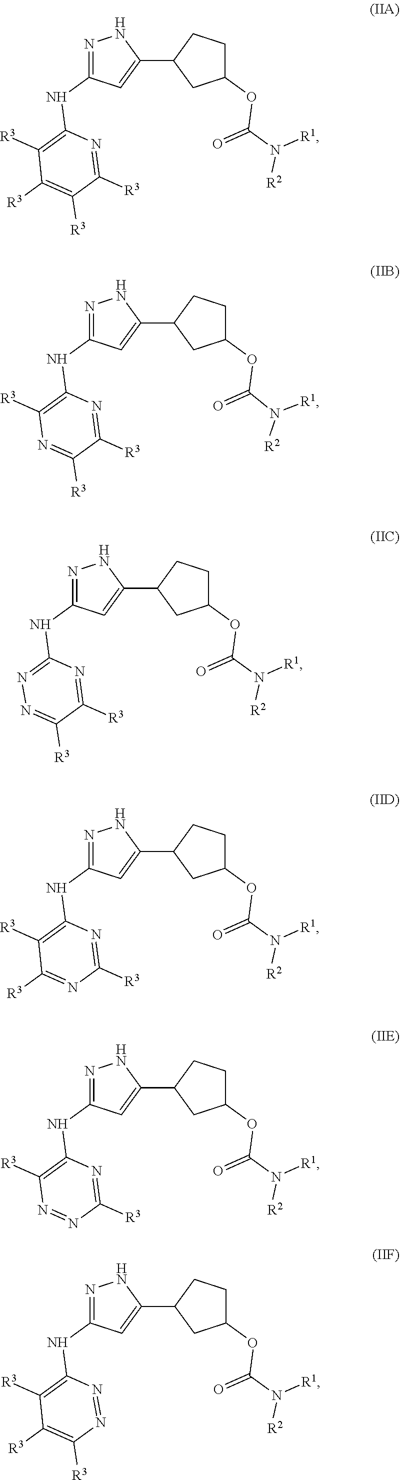
or a pharmaceutically acceptable salt thereof.

4. The compound of any one of claims 1-3, wherein R¹ is C₁-C₆alkyl and R² is H.

5. The compound of claim 1 or 3, wherein R₁ is C₁-C₆alkyl optionally substituted with 1 to 4 groups selected from halo and C₃-C₆cycloalkyl and R₂ is H.

6. The compound of any one of claims 1-3, wherein R¹ is C₃₋₄cycloalkyl, optionally substituted by methyl.

7. The compound of any one of claims 1-3, wherein R¹ is oxetanyl, tetrahydrofuranyl, tetrahydropyranyl, azetidinyl, pyrrolidinyl, or piperdinyl optionally substituted by methyl or CF₃.

8. The compound of claim 6, wherein the compound is selected from the group consisting of Formula IIIA, IIIB, IIIC, IIID, IIIE and IIIF: 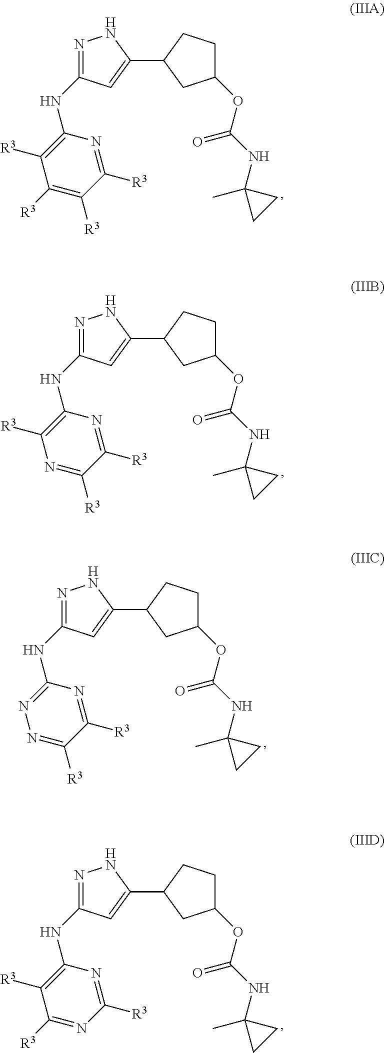 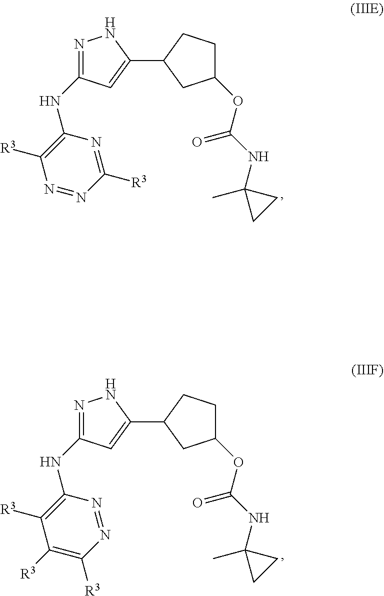
or a pharmaceutically acceptable salt thereof.

9. The compound of any one of claims 1-3, wherein R¹ and R², together with the nitrogen to which they are attached, form a 4-5 membered heterocyclyl, optionally substituted on a free carbon by one or two methyl groups.

10. The compound of claim 1 or 3, wherein R¹ and R², together with the nitrogen to which they are attached, form a 4-6 membered heterocyclyl, wherein the heterocyclyl optionally includes 1 or 2 heteroatoms independently selected from O, N, or S in addition to the nitrogen atom attached to R¹ and R², and is optionally substituted on a free carbon or nitrogen by CN, CO₂C(CH₃)₃, OCH₃, or one or two methyl groups optionally substituted with 1 to 4 halo.

11. The compound of claim 9 or 10, wherein the structure is selected from the group consisting of Formula IVA, IVB, IVC, IVD, IVE and IVF: 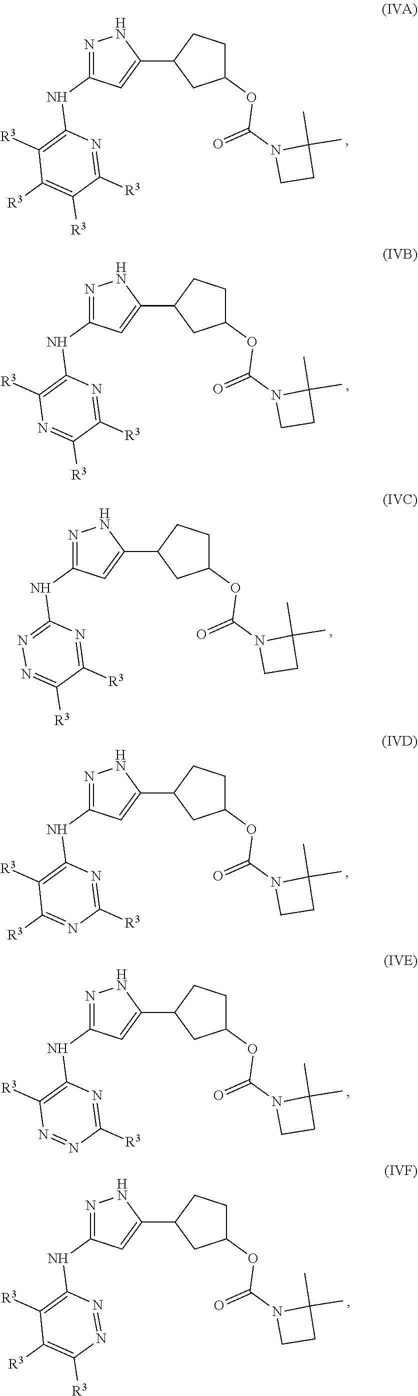
or a pharmaceutically acceptable salt thereof.

12. The compound of any one of claims 1 to 11 wherein
each R³ is independently selected from the group consisting of H, —OH, halo, CN, NR^{a}R^{b}, SO₂R^{c}, C₁-C₄alkyl, and C₁-C₄alkoxyl, wherein the alkyl and alkoxy are each optionally substituted with 1 to 4 groups independently selected from halo, OR^{3a}, or cyclopropyl;
each R^{a} is independently selected for each occurrence from the group consisting of H, methyl, or ethyl;
each R^{b} is independently selected for each occurrence from methyl and ethyl;
R^{3a} is independently selected for each occurrence from methyl or ethyl; and
R^{c} is selected for each occurrence from methyl or ethyl.

13. A pharmaceutical composition comprising a pharmaceutically acceptable carrier and a compound of any one of claims 1-12, or a pharmaceutically acceptable salt thereof.

14. A method of treating a cancer in a subject in need thereof, comprising administering to the subject an effective amount of a compound of any of claims 1-12, or a pharmaceutically acceptable salt thereof, or the pharmaceutical composition of claim 13.

15. The method of claim 14, wherein the cancer is at least one of: uterine cancer (including uterine carcinosarcoma, uterine corpus endometrial carcinoma), endometrial cancer, breast cancer (including breast invasive carcinoma, TNBC (triple negative breast cancer), ER (estrogen receptor)+HER2 (human epidermal growth factor 2)− breast cancer, and HER2+ breast cancer), ovarian cancer (e.g., ovarian serous cystadenocarcinoma), stomach cancer (including stomach adenocarcinoma), gastric cancer (including gastrointestinal stromal tumor), colorectal cancer, pancreatic cancer, kidney cancer, head and neck cancer, liver cancer, prostate cancer, skin cancer, lymphoma (including B-cell lymphoma), sarcoma, esophageal cancer (including esophageal carcinoma), bladder cancer (including bladder urothelial carcinoma), lung cancer (including lung squamous carcinoma and non-small cell lung cancer, e.g., EGFRm (epidermal growth factor receptor mutant)+ non-small cell lung cancer), cholangiocarcinoma, adrenocortical carcinoma, or mesothelioma.

16. A method of inhibiting CDK2, comprising administering to a subject in need thereof an effective amount of the compound of any of claims 1-12, or a pharmaceutically acceptable salt thereof, or the pharmaceutical composition of claim 13.

17. A method of treating a subject having, or at risk of developing, a disease or disorder associated with CDK2, comprising administering to the subject a therapeutically effective amount of a compound of any one of claims 1-12, or a pharmaceutically acceptable salt thereof, or a pharmaceutical composition of claim 13, wherein the subject has an amplification of the CCNE1 gene and/or has an expression level of CCNE1 higher than a control expression level of CCNE1.

18. The method of claim 17, wherein the disease or disorder associated with CDK2 is cancer.

19. A method of treating a patient having an amplified expression level of CCNE1 and suffering from, or at risk of developing, a cancer, comprising administering to the patient a therapeutically effective amount of a compound of any one of claims 1-12, or a pharmaceutically acceptable salt thereof, or a pharmaceutical composition of claim 13.

20. The method of claim 19, wherein the cancer is at least one of: uterine cancer (including uterine carcinosarcoma, uterine corpus endometrial carcinoma), endometrial cancer, breast cancer (including breast invasive carcinoma, TNBC (triple negative breast cancer), ER (estrogen receptor)+HER2 (human epidermal growth factor 2)− breast cancer, and HER2+ breast cancer), ovarian cancer (e.g., ovarian serous cystadenocarcinoma), stomach cancer (including stomach adenocarcinoma), gastric cancer (including gastrointestinal stromal tumor), colorectal cancer, pancreatic cancer, kidney cancer, head and neck cancer, liver cancer, prostate cancer, skin cancer, lymphoma (including B-cell lymphoma), sarcoma, esophageal cancer (including esophageal carcinoma), bladder cancer (including bladder urothelial carcinoma), lung cancer (including lung squamous carcinoma and non-small cell lung cancer, e.g., EGFRm (epidermal growth factor receptor mutant)+ non-small cell lung cancer), cholangiocarcinoma, adrenocortical carcinoma, or mesothelioma.
